SEQUENCE LISTING (1 sequence)

SEQ ID NO: 1 - DNA, 19 bp, Artificial Sequence
  RNaseP forward primer
agatttggacctgcgagcg

(19) United States
(12) Patent Application Publication
(10) Pub. No.: US 2016/0199491 A1
(43) Pub. Date: Jul. 14, 2016

(54) Compounds compositions and methods including thermally labile moieties

(71) Applicant: Biosearch Technologies, Inc., Petaluma, CA (US)

(72) Inventors: Ronald Michael Cook, Novato, CA (US); Brett Michael Cook, Goleta, CA (US); Matthew H. Lyttle, Sonoma, CA (US)

(73) Assignee: Biosearch Technologies, Inc., Petaluma, CA (US)

(21) Appl. No.: 14/545,280

(22) Filed: Apr. 15, 2015

Related U.S. Application Data
(60) Provisional application No. 61/996,092, filed on Apr. 29, 2014.

(51) Int. Cl. A61K 41/00; A61K 31/7076; C07H 19/10; B01J 19/24; A61K 31/708; A61K 31/7072; C12N 15/113; C12P 19/34; C07H 19/20; A61K 31/7068

(52) U.S. Cl. CPC A61K 41/0042; C07H 19/20; A61K 31/7076; C07H 19/10; A61K 31/7068; A61K 31/708; A61K 31/7072; C12N 15/113; C12P 19/34; B01J 19/24; C12N 2310/314; C12N 2330/30; B01J 2219/00051

(57) ABSTRACT

The present invention generally relates to compounds that include one or more thermally labile protecting groups, compositions including the compounds, methods of making the compounds and compositions and methods of using the compounds and compositions. In one aspect, the present invention is directed to a compound of the structure XO—CH₂—SM-B-A. The substituent X is H, an acid labile protecting group, a solid support, —P(O—R¹)NR²R³, —P(O)(OH)H, —P(O)(OR¹)H, —P(O)(OH)₂, —P(O)(OH)O—P(O)(OH)OP(O)(OH)₂ or salts thereof. The substituent R¹ is CNE (i.e., cyanoethyl), alkyl, or heteroalkyl and R² and R³ are independently alkyl. The substituent SM is a sugar moiety or analogue thereof that is not a natural furanosyl, B is a base moiety or analogue thereof, and A is a moiety attached to a nitrogen on or in the base moiety of the structure —C(O)OR⁴, wherein R⁴ is tertiary alkyl.

### REFERENCE TO SEQUENCE LISTING

This application includes a sequence listing in .txt format submitted on compact disc. The .txt file contains a sequence listing entitled “2015-06-09 BT-001.02_ST25.txt” created on Jun. 9, 2015 and is 605 bytes in size. The sequence listing contained in this .txt file is part of the specification and is hereby incorporated by reference herein in its entirety.

### FIELD OF THE INVENTION

The present invention generally relates to compounds that include one or more thermally labile moieties, compositions including the compounds, methods of making the compounds and compositions and methods of using the compounds and compositions.

### BACKGROUND OF THE INVENTION

Molecular moieties that can be removed under mild conditions are of importance with respect to the synthesis and action of a wide range of compounds. Scientists have accordingly performed substantial research directed to the discovery and use of such compounds, including work directed to protecting groups used in complex synthetic methods.

For example, U.S. Pat. No. 5,614,622 entitled, “5-Pentenoyl moiety as a nucleoside-amino protecting group, 4-pentenoyl-protected nucleotide synthons, and related oligonucleotide syntheses” was issued on Mar. 25, 1997. The discussed invention of the patent is allegedly directed to the following: “The invention provides new methods for synthesizing oligonucleotides that allow for deprotection of the oligonucleotide under more mild conditions than existing methods. The invention further provides a nucleoside base protective group that is stable under oligonucleotide synthesis conditions, but which can be removed under more mild conditions than existing protective groups, as well as nucleoside synthons having such base protective groups.” Abstract.

Another example, U.S. Pat. No. 6,762,298 entitled, “Thermolabile phosphorus protecting groups, associated intermediates and methods of use” was issued on Jul. 13, 2004. The discussed invention of the patent is allegedly directed to the following: “The invention provides a method of thermally deprotecting the internucleosidic phosphorus linkage of an oligonucleotide, which method comprises heating a protected oligonucleotide in a fluid medium at a substantially neutral pH, so as to deprotect the oligonucleotide. The present invention further provides a method of synthesizing an oligonucleotide using the thermal deprotection method described above, and novel oligonucleotides and intermediates that incorporate the thermolabile protecting group used in accordance with the present invention.” Abstract.

Another example, U.S. Pat. No. 7,355,037 entitled, “Thermolabile hydroxyl protecting groups and methods of use” was issued on Apr. 8, 2008. The discussed invention of the patent is allegedly directed to the following: “Provided is a hydroxyl-protected alcohol of the formula R-O-Pg, wherein Pg is a protecting group of the formula:

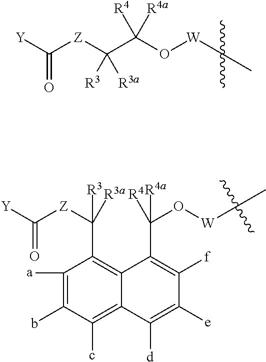

wherein Y, Z, W, R¹, R^{1a}, R², R^{2a}, R³, R^{3a}, R⁴, R^{4a}, a, b, c, d, e and fare defined herein and R is a nucleosidyl group, an oligonucleotidyl group with 2 to about 300 nucleosides, or an oligomer with 2 to about 300 nucleosides. Also provided is a deprotection method, which includes heating the hydroxyl-protected alcohol at a temperature effective to cleave thermally the hydroxyl-protecting group therefrom.” Abstract.

Another example, U.S. Pat. No. 8,133,669 entitled, “Chemically modified nucleoside 5′-triphosphates for thermally initiated amplification of nucleic acid” was issued on Mar. 13, 2012. The discussed invention of the patent is allegedly directed to the following: “Provided herein are methods and compositions for nucleic acid replication. These methods involve the use of 3′-substituted nucleoside 5′-triphosphates or 3′-substituted terminated primers in nucleic acid replication reactions. In certain aspects, the methods are accomplished by use of 3′-substituted NTPs and/or 3′-substituted terminated primers which provide utility in nucleic acid replication. In preferred embodiments, the NTPs and/or primers are substituted at the 3′-position with particular heat labile chemical groups such as ethers, esters or carbonate esters.” Abstract.

Despite the research that has been performed on molecular moieties that can be removed under mild conditions, there is still a need in the art for new molecular moieties, as well as related compositions and methods.

### SUMMARY OF THE INVENTION

In one aspect, the present invention is directed to a compound of the structure XO—CH₂-SM-B-A. The substituent X is H, an acid labile protecting group, a solid support, —P(O—R¹)NR²R³, —P(O)(OH)H, —P(O)(OR¹)H, —P(O)(OH)₂, —P(O)(OH)O—P(O)(OH)OP(O)(OH)₂ or salts thereof. The substituent R¹ is CNE, alkyl, or heteroalkyl and R² and R³ are independently alkyl. The substituent SM is a sugar moiety or analogue thereof that is not a natural furanosyl, B is a base moiety or analogue thereof, and A is a moiety attached to a nitrogen on or in the base moiety of the structure —C(O)OR⁴, wherein R⁴ is tertiary alkyl.

### BRIEF DESCRIPTION OF THE FIGURES

FIG. 1 is a schematic of an instrument used to synthesize polymers (e.g., DNA oligomers).

FIG. 2 shows the fraction of Boc remaining from 5′ dC(Boc) T10 3′ in neutral water over a 12 min time course.

FIG. 3 shows the fraction of Boc remaining from 5′dA(t-Boc)T10 at 94° C. over a 15 min time course.

FIG. 4 shows the fraction of Boc remaining from rC(BOC)-T10 at 94° C. over a 15 min time course.

FIG. 5 shows the fraction of Boc remaining from rA(BOC)-T10 at 94° C. over a 15 min time course.

FIG. 6 shows an HPLC before heating of a Boc-protected PCR primer.

FIG. 7 shows an ESMS before heating of a Boc-protected PCR primer.

FIG. 8 shows an HPLC after heating of a Boc-protected PCR primer.

FIG. 9 shows an ESMS after heating of a Boc-protected PCR primer.

### DETAILED DESCRIPTION OF THE INVENTION

The present invention generally relates to compounds that include one or more thermally labile protecting groups, compositions including the compounds, methods of making the compounds and compositions and methods of using the compounds and compositions.

A “Linker” is typically an alkyl, substituted alkyl, heteroalkyl, substituted heteroalkyl, aryl, substituted aryl, heteroaryl or substituted heteroaryl terminating at both ends with either an electrophilic or nucleophilic functional group. Nonlimiting examples of such functional groups include: —C(O)—, —C(O)N(H)—, —C(O)N(R²¹)—, —C(O)O—, —N(R²²)—, —O—, —S—, where R²¹ and R²² are, independently, alkyl, substituted alkyl, heteroalkyl, substituted heteroalkyl, aryl or substituted aryl. Nonlimiting examples of Linkers include: —C(O)CH₂OC₆H₅OCH₂C(O)—, —C(O)—(CH₂)ₙ—C(O) where n is 0, 1, 2, 3, 4 or 5; —C(O)—(CH₂)ₙ—N(H)— where n is 1, 2, 3, 4 or 5; —C(O)—(CH₂)ₙ—O— where n is 1, 2, 3, 4 or 5; and, —N(H)—(CH₂)ₙ—N(H)— where n is 1, 2, 3, 4 or 5.

A “Label” is a moiety that is capable of being detected (e.g., optically, electronically, magnetically, and chemically). Nonlimiting examples of Label categories include: fluorescent dyes; fluorescent quenching molecules; chelating agents for metal coordination; membrane soluble agents (e.g., cholesterol); intercalating agents (e.g., acridine); DNA minor groove binders; and, azides and alkynes (e.g., Click chemistry).

Nonlimiting examples of fluorescent dye types include: acridine dyes; cyanine dyes (e.g., SYBR green); fluorone dyes (e.g., fluorescein); oxazine dyes (e.g., Nile blue, Nile red); phenanthridine dyes; and rhodamine dyes (e.g., Texas Red). Nonlimiting examples of fluorescent dyes include: FAM; TET; Alexa Fluor 488; CAL Fluor Gold 540; HEX; CAL Fluor Orange 560; Quasar 470; 5-TAMRA; CA L Fluor Red 590; Cy3; T(Rox); CAL Fluor Red 610; CAL Fluor Red 635; T(JOE); Cy5; Quasar 670; Quasar 705.

Nonlimiting examples of fluorescent quenching molecules include: BHQ-1; BHQ-2; DABCYL; Pulsar 650.

A “solid support” is a material used in solid phase polymer synthesis. Typically a monomer, either directly or through a linker, is covalently bound to the solid support and the polymer chain is grown on the solid support through subsequent addition of other monomers. Oligonucleotide synthesis proceeds best on non-swellable or low-swellable solid supports. The solid supports used most often for oligonucleotide synthesis are controlled pore glass (CPG) and polystyrene (e.g., macroporous polystyrene).

A “phosphorus containing moiety” is chemical group containing at least one phosphorus atom. Nonlimiting examples of phosphorus containing moieties include: —P(OR²³)NR²⁴R²⁵; —P(═O)(OR²³)NR²⁴R²⁵; —P(OH)₂; —P(OR²³)OH; —P(O)(OR²³)OH; —P(O)(OH)₂; —P(O)(OH)OP(O)(OH)₂; —P(O)(OH)OP(O)(OH)OP(O)(OH)₂; —P(S)(OH)₂; and salts of the preceding compounds. R²³ is alkyl (e.g., —CH₃), substituted alkyl (e.g., —CH₂CH₂-EWG, where “EWG” is an electron withdrawing group such as —CN or -Ph-NO₂), heteroalkyl, substituted heteroalkyl, aryl, substituted aryl, heteroaryl, or substituted heteroaryl. R²⁴ and R²⁵ are independently alkyl, substituted alkyl, heteroalkyl, substituted heteroalkyl, aryl, substituted aryl, heteroaryl, or substituted heteroaryl; or combine to form a cyclic, fused, fused cyclic or heterocyclic ring.

For a discussion of phosphorus reagents, see: Beaucage, S. L.; Caruthers M. H. (1981). “Deoxynucleoside phosphoramidites—A new class of key intermediates for deoxypolynucleotide synthesis”. Tetrahedron Letters 22: 1859-1862; Lin, K.-Y., Matteucci, M. D. (1998). “A cytosine analog capable of clamp-like binding to a guanine in helical nucleic acids”. J. Amer. Chem. Soc. 120 (33): 8531-8532; Nielsen, J.; Marugg, J. E.; Taagaard, M.; Van Boom, J. H.; Dahl, O. (1986). “Polymer-supported synthesis of deoxyoligonucleotides using in situ prepared deoxynucleoside 2-cyanoethyl phosphoramidites”. Rec. Trav. Chim. Pays-Bas 105 (1): 33-34; Nielsen, J.; Taagaard, M.; Marugg, J. E.; Van Boom, J. H.; Dahl, 0. (1986). “Application of 2-cyanoethyl N,N,N′,N′-tetraisopropylphosphorodiamidite for in situ preparation of deoxyribonucleoside phosphoramidites and their use in polymer-supported synthesis of oligodeoxyribonucleotides”. Nucl. Acids Res. 14 (18): 7391-7403; Nielsen, J.; Marugg, J. E.; Van Boom, J. H.; Honnens, J.; Taagaard, M.; Dahl, 0. (1986). “Thermal instability of some alkyl phosphorodiamidites”. J. Chem Res. Synopses (1): 26-27; Nielsen, J.; Dahl, O. (1987). “Improved synthesis of 2-cyanoethyl N,N,N′,N′-tetraisopropylphosphorodiamidite (iPr2N)2POCH2CH2CN)”. Nucl. Acids Res. 15 (8): 3626; Beaucage, S. L. (2001). “2-Cyanoethyl Tetraisopropylphosphorodiamidite”. e-EROS Encyclopedia of Reagents for Organic Synthesis; Sinha, N. D.; Biernat, J.; Koester, H. (1983). “β-Cyanoethyl N,N-dialkylamino/N-morpholinomonochloro phosphoamidites, new phosphitylating agents facilitating ease of deprotection and work-up of synthesized oligonucleotides”. Tetrahedron Lett. 24 (52): 5843-5846; Marugg, J. E.; Burik, A.; Tromp, M.; Van der Marel, G. A.; Van Boom, J. H. (1986). “A new and versatile approach to the preparation of valuable deoxynucleoside 3′-phosphite intermediates”. Tetrahedron Lett. 24 (20): 2271-22274; Guzaev, A. P.; Manoharan, M. (2001). “2-Benzamidoethyl group—a novel type of phosphate protecting group for oligonucleotide synthesis”. J. Amer. Chem. Soc. 123 (5): 783-793; Sproat, B.; Colonna, F.; Mullah, B.; Tsou, D.; Andrus, A.; Hampel, A.; Vinayak, R. (February 1995). “An efficient method for the isolation and purification of oligoribonucleotides”. Nucleosides & Nucleotides 14 (1&2): 255-273; Stutz, A.; Hobartner, C.; Pitsch, S. (September 2000). “Novel fluoride-labile nucleobase-protecting groups for the synthesis of 3′(2′)-O-amino-acylated RNA sequences”. Helv. Chim. Acta 83 (9): 2477-2503; Welz, R.; Muller, S. (January 2002). “5-(Benzylmercapto)-1H-tetrazole as activator for 2′-O-TBDMS phosphoramidite building blocks in RNA synthesis”. Tetrahedron Letters 43 (5): 795-797; Vargeese, C.; Carter, J.; Yegge, J.; Krivjansky, S.; Settle, A.; Kropp, E.; Peterson, K.; Pieken, W. (1998). Nucl. Acids Res. 26 (4): 1046-1050; Gacs-Baitz, E.; Sipos, F.; Egyed, O.; Sagi, G. (2009). “Synthesis and structural study of variously oxidized diastereomeric 5′-dimethoxytrityl-thymidine-3′-O-[O-(2-cyanoethyl)-N,N-diisopropyl]-phosphoramidite derivatives. Comparison of the effects of the P═O, P═S, and P═Se functions on the NMR spectral and chromatographic properties.”. Chirality 21 (7): 663-673; M. J.; Ogilvie, K. K. (1980). “Phosphoramidate analogs of diribonucleoside monophosphates.”. Tetrahedron Lett. 21 (43): 4153-4154; Wilk, A.; Uznanski, B.; Stec, W. J. (1991). “Assignment of absolute configuration at phosphorus in dithymidylyl(3′,5′)phosphormorpholidates and -phosphormorpholidothioates.”. Nucleosides & Nucleotides 10 (1-3): 319-322. The preceding references are hereby incorporated-by-reference into this document for all purposes.

A “protecting group” is a chemical moiety typically used to mask a reactive functional group during synthetic manipulations. Nonlimiting categories of protecting groups include: acid labile protecting groups; base labile protecting groups; reductively labile protecting groups; photolabile protecting groups; and, thermally labile protecting groups.

Nonlimiting examples of acid labile protecting groups include: trityl; monomethoxytrityl; 4,4′-dimethoxytrityl (DMT); β-methoxyethoxymethyl ether (MEM); methoxymethyl ether (MOM); methylthiomethyl ether; tetrahydropyranyl (THP); 4-methoxytetrahydropyran-4-yl; tetrahydrofuranyl (THF); tert-butyloxycarbonyl (Boc); silyl ethers (e.g., trimethylsilyl (TMS), tert-butyldimethylsilyl (TBDMS), triisopropylsilyloxymethyl (TOM). The silyl ethers are also fluoride ion labile.

Nonlimiting examples of base labile protecting groups include: benzoyl and other arylcarboxylate derivatives; acetyl and other alkylcarboxylate derivatives; alkyl- or aryloxyacetates; trihaloacetate; dihaloacetate; acyloxymethyl ethers; fluorenylmethyloxycarbonyl (FMOC); cyanoethyl; substituted alkyl groups such as —CH₂CH₂-EWG, where “EWG” is an electron withdrawing group such as -PhNO₂ or —C(O)—; cyanoethyloxycarbonyl. Nonlimiting examples of reductively labile protecting groups include: benzyl and substituted analogues; benzyloxycarbonyl (Z); allyloxycarbonyl. Nonlimiting examples of photolabile protecting groups include: o-nitrobenzyl ether and substituted derivatives; o-nitrobenzylcarbamate. Nonlimiting examples of thermally labile protecting groups include: tert-butyloxyethyl ether (hydroxyl groups); 4-oxoalkyl esters; 3-acylaminopropyl esters; amides and esters of 4-carboxypropyl esters; 5-alkylthioalkyl esters.

An “alkyl” is a chemical moiety having the general formula CₙH₂ₙ₊₁. Alkyl groups are typically of the following categories: lower alkyl; higher alkyl; cyclic alkyl; and, branched alkyl. A lower alkyl group has six or fewer carbon atoms. Nonlimiting examples include: methyl; ethyl; propyl; butyl; and pentyl. A higher alkyl has seven or more carbon atoms. Nonlimiting examples include: heptyl; octyl; nonyl. A cyclic alkyl is an alkyl forming a ring structure and is of the formula CₙH₂ₙ₋₁. Nonlimiting examples include: cyclopropyl; cyclobutyl; cyclopentyl; and cyclohexyl. A branched alkyl is an alkyl chain (i.e., linear) where one or more of the hydrogen atoms is substituted with an alkyl group. Nonlimiting examples include: iso-propyl; sec-butyl; and tert-butyl.

A “heteroalkyl” is an alkyl where one or more of the carbon atoms is replaced by a heteroatom (e.g., O, S, NH). Nonlimiting examples include: —CH₂OCH₃; —CH₂CH₂OCH₃; —NC₄H₈O (morpholino).

A “substituted alkyl” is an alkyl where one or more of the hydrogen atoms is replaced by a functional group. Nonlimiting examples of functional groups include the following, where R²⁶, R²⁷, and R²⁸ are independently alkyl, substituted alkyl, heteroalkyl, substituted heteroalkyl, aryl, substituted aryl, heteroaryl or substituted heteroaryl: —OH; —SH; —NH₂; —OCH₃; —OCH₂CH₃; —SCH₃; —NHR²⁶; —NR²⁷R²⁸; —NO₂; —CN; —CO₂H; —C(O)OR²⁹; —OC(O)OR²⁹; —C(O)NH₂; —C(O)NHR²⁶; —C(O)NR²⁶R²⁷; —OC(O)NHR²⁶; —OC(O)NR²⁶R²⁷; —NHC(O)NHR²⁶; —NHC(O)NR²⁶R²⁷, where R²⁶, R²⁷, R²⁸ and R²⁹ are, independently, alkyl, substituted alkyl, aryl or substituted aryl; —F; —Cl; —Br; —I; —Ar, where “Ar” is an aryl group; —Ar—X, where “Ar—X” is a substituted aryl group; —HAr, where “—HAr” is a heteroaryl group; and, —HAr—X where “—HAr—X” is a substituted heteroaryl group.

A “substituted heteroalkyl” is a heteroalkyl where one or more of the hydrogen atoms is replaced by a functional group, where R³⁰, R³¹, R³² and R³³ are independently alkyl, substituted alkyl, heteroalkyl, substituted heteroalkyl, aryl, substituted aryl, heteroaryl or substituted heteroaryl: —OH; —SH; —NH₂; —OCH₃; —OCH₂CH₃; —SCH₃; —NHR³⁰; —NR³¹R³²; —NO₂; —CN; —CO₂H; —C(O)OR³³; —OC(O)OR³³; —C(O)NH₂; —C(O)NHR³⁰; —C(O)NR³¹R³²; —OC(O)NHR³¹; —OC(O)NR³¹R³²; —NHC(O)NHR³¹; —NHC(O)NR³¹R³²; —F; —Cl; —Br; —I.

An “aryl” group is of the structure:

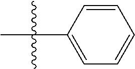 [SMILES: *c1ccccc1]

A “substituted aryl” group is of the structure:

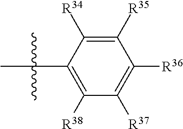

Wherein R³⁴, R³⁵, R³⁶, R³⁷ and R³⁸ are independently selected from H, alkyl, substituted alkyl, heteroalkyl, substituted heteroalkyl, aryl, substituted aryl, heteroaryl, substituted heteroaryl, —OH; —SH; —NH₂; —OCH₃; —OCH₂CH₃; —SCH₃; —NHR³⁹; —NR⁴⁰R⁴¹; —NO₂; —CN; —CO₂H; —C(O)OR⁴²; —OC(O)OR⁴²; —C(O)NH₂; —C(O)NHR³⁹; —C(O)NR⁴⁰R⁴¹; —OC(O)NHR³⁹; —OC(O)NR⁴⁰R⁴¹; —NHC(O)NHR³⁹; —NHC(O)NR⁴⁰R⁴¹; —F; —Cl; —Br; —I; where R³⁹, R⁴⁰, R⁴¹ and R⁴² are independently selected from alkyl, substituted alkyl, heteroalkyl, substituted heteroalkyl, aryl, substituted aryl, heteroaryl or substituted heteroaryl; provided that at least one of R³⁴, R³⁵, R³⁶, R³⁷, and R³⁸ is not H.

A “heteroaryl” group is an aromatic heterocycle. Nonlimiting examples of heteroaryl groups include:

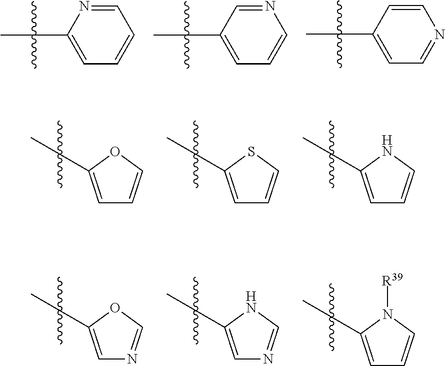

where R³⁹ is selected from alkyl, substituted alkyl, aryl and substituted aryl.

A “substituted heteroaryl” group is a heteroaryl group having one or more substituents selected from H, alkyl, substituted alkyl, heteroalkyl, substituted heteroalkyl, aryl, substituted aryl, heteroaryl, substituted heteroaryl, —OH; —SH; —NH₂; —OCH₃; —OCH₂CH₃; —SCH₃; —NHR⁴³; —NR⁴⁴R⁴⁵; —NO₂; —CN; —CO₂H; —C(O)OR⁴⁶; —OC(O)OR⁴⁶; —C(O)NH₂; —C(O)NHR⁴³; —C(O)NR⁴⁴R⁴⁵; —OC(O)NHR⁴³; —OC(O)NR⁴⁴R⁴⁵; —NHC(O)NHR⁴³; —NHC(O)NR⁴⁴R⁴⁵; —F; —Cl; —Br; —I; where R⁴³, R⁴⁴, R⁴⁵ and R⁴⁶ are independently selected from alkyl, substituted alkyl, heteroalkyl, substituted heteroalkyl, aryl, substituted aryl, heteroaryl or substituted heteroaryl.

Compounds of the invention are of the structure XO—CH₂—SM-B-A. Substituent “X” is H, a protecting group, a solid support which optionally includes a linker between the oxygen and the solid support, a phosphorus containing moiety or salts thereof. “SM” is a sugar moiety or an analogue of a sugar moiety. “B” is a nucleobase moiety or an analogue of a nucleobase moiety. “A” is one or more moieties attached to one or more nitrogen atoms on or within the base moiety and is of the structure —C(O)OR¹, where R¹ is a tertiary alkyl group.

For a discussion of nucleosides synthesis, see: Vorbrüggen, H.; Ruh-Polenz, C. Org. React. 2000, 55, 1; Diekmann, E.; Friedrich, K.; Fritz, H.-G. J. Prakt. Chem. 1993, 335, 415; Fischer, E.; Helferich, B. Chem. Ber. 1914, 47, 210; Miyaki, M.; Shimizu, B. Chem. Pharm. Bull. 1970, 18, 1446; Kazimierczuk, Z.; Cottam, H. B.; Revankar, G. R.; Robins, R. K. J. Am. Chem. Soc. 1984, 106, 6379; Wittenburg, E. Z. Chem. 1964, 4, 303; Choi, W-B.; Wilson, L. J.; Yeola, S.; Liotta, D. C.; Schinazi, R. F. J. Am. Chem. Soc. 1991, 113, 9377; Vorbrüggen, H.; Niedballa, U.; Krolikiewicz, K.; Bennua, B.; Höfle, G. In Chemistry and Biology of Nucleosides and Nucleotides; Harmon, R. E., Robins, R. K., Townsend, L. B., Eds.; Academic: New York, 1978; p. 251; Prystas, M.; \check{S}orm, F. Collect. Czech. Chem. Commun. 1964, 29, 121; Niedballa, U.; Vorbrüggen, H. J. Org. Chem. 1974, 39, 3668; Itoh, T.; Melik-Ohanjanian, R. G.; Ishikawa, I.; Kawahara, N.; Mizuno, Y.; Honma, Y.; Hozumi, M.; Ogura, H. Chem. Pharm. Bull. 1989, 37, 3184; Vorbrüggen, H.; Bennua, B. Tetrahedron Lett. 1978, 1339; Vorbrüggen, H.; Bennua, B. Chem. Ber. 1981, 114, 1279; Sugiura, Y.; Furuya, S.; Furukawa, Y. Chem. Pharm. Bull. 1988, 36, 3253; Kawasaki, A. M.; Wotring, L. L.; Townsend, L. B. J. Med. Chem. 1990, 33, 3170; Nair, V.; Purdy, D. F. Heterocycles 1993, 36, 421; Hanrahan, J. R.; Hutchinson, D. W. J. Biotechnol. 1992, 23, 193; Martin, O. R. Tetrahedron Lett. 1985, 26, 2055; Langer, S. H.; Connell, S.; Wender, I. J. Org. Chem. 1958, 23, 50; Patil, V. D.; Wise, D. S.; Townsend, L. B. J. Chem. Soc., Perkin Trans. 1 1980, 1853; Vorbrüggen, H.; Krolikiewicz, K.; Bennua, B. Chem. Ber. 1981, 114, 1234. The preceding references are hereby incorporated-by-reference into this document for all purposes.

A sugar moiety is typically a pentofuranosyl moiety. Nonlimiting examples of such moieties include (where XOCH₂—, B and A of the compounds are shown):

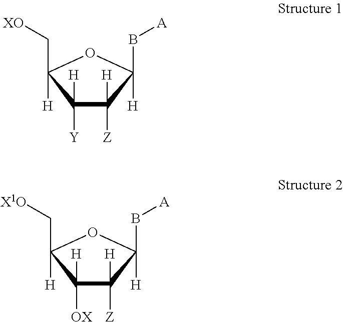

where the substituents of Structure 1 and Structure 2 above are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; Y is OH or OR² where R² is a protecting group, an alkyl, a substituted alkyl, a heteroalkyl, a substituted heteroalkyl, an aryl or a substituted aryl; and, Z is H, OH or OR³ where R³ is a protecting group, an alkyl, a substituted alkyl, a heteroalkyl, a substituted heteroalkyl, an aryl or a substituted aryl.

An analogue of a sugar moiety is typically an analogue of a natural furanosyl moiety. Nonlimiting examples of such moieties include:

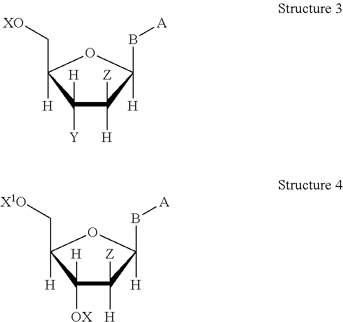

where the substituents of Structure 3 and Structure 4 above are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; Y is OH or OR² where R² is a protecting group, an alkyl, a substituted alkyl, a heteroalkyl, a substituted heteroalkyl, an aryl or a substituted aryl; and, Z is H, OH or OR³ where R³ is a protecting group, an alkyl, a substituted alkyl, a heteroalkyl, a substituted heteroalkyl, an aryl or a substituted aryl.

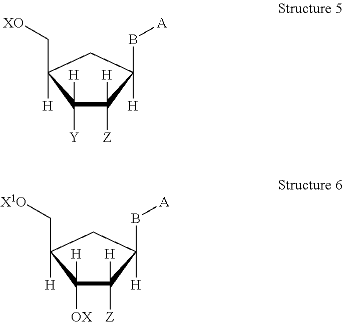

where the substituents of Structure 5 and Structure 6 above are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; Y is OH or OR² where R² is a protecting group, an alkyl, a substituted alkyl, a heteroalkyl, a substituted heteroalkyl, an aryl or a substituted aryl; and, Z is H, OH or OR³ where R³ is a protecting group, an alkyl, a substituted alkyl, a heteroalkyl, a substituted heteroalkyl, an aryl or a substituted aryl.

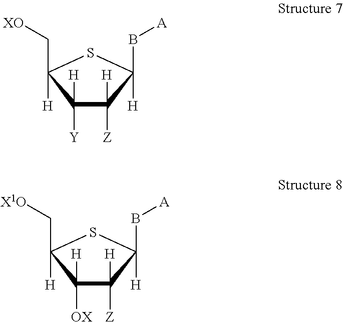

where the substituents of Structure 7 and Structure 8 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; Y is OH or OR² where R² is a protecting group, an alkyl, a substituted alkyl, a heteroalkyl, a substituted heteroalkyl, an aryl or a substituted aryl; and, Z is H, OH or OR³ where R³ is a protecting group, an alkyl, a substituted alkyl, a heteroalkyl, a substituted heteroalkyl, an aryl or a substituted aryl.

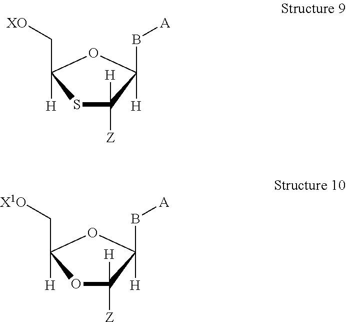

where the substituents of Structure 9 and Structure 10 above are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); and, Z is H, OH or OR³ where R³ is a protecting group, an alkyl, a substituted alkyl, a heteroalkyl, a substituted heteroalkyl, an aryl or a substituted aryl.

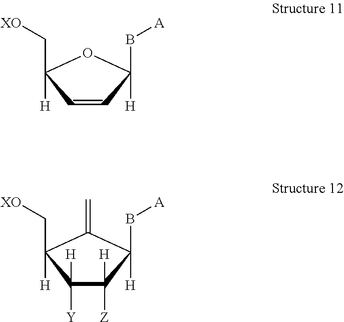

where the substituents of Structure 11 and Structure 12 above are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); Y is OH or OR² where R² is a protecting group, an alkyl, a substituted alkyl, a heteroalkyl, a substituted heteroalkyl, an aryl or a substituted aryl; and, Z is H, OH or OR³ where R³ is a protecting group, an alkyl, a substituted alkyl, a heteroalkyl, a substituted heteroalkyl, an aryl or a substituted aryl.

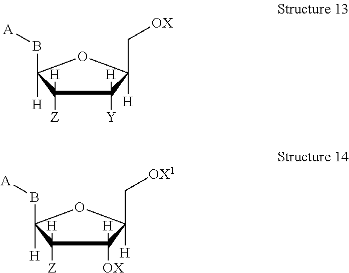

where the substituents of Structure 13 and Structure 14 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; Y is OH or OR² where R² is a protecting group, an alkyl, a substituted alkyl, a heteroalkyl, a substituted heteroalkyl, an aryl or a substituted aryl; and, Z is H, OH or OR³ where R³ is a protecting group, an alkyl, a substituted alkyl, a heteroalkyl, a substituted heteroalkyl, an aryl or a substituted aryl.

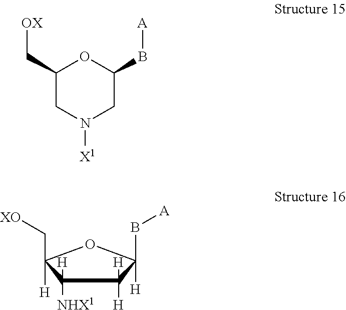

where the substituents of Structure 15 and Structure 16 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; Y is OH or OR² where R² is a protecting group, an alkyl, a substituted alkyl, a heteroalkyl, a substituted heteroalkyl, an aryl or a substituted aryl; and, Z is H, OH or OR³ where R³ is a protecting group, an alkyl, a substituted alkyl, a heteroalkyl, a substituted heteroalkyl, an aryl or a substituted aryl.

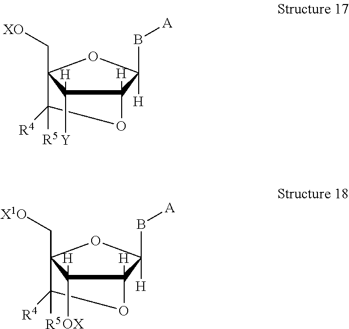

where the substituents of Structure 17 and Structure 18 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; Y is OH or OR² where R² is a protecting group, an alkyl, a substituted alkyl, a heteroalkyl, a substituted heteroalkyl, an aryl or a substituted aryl; and, R⁴ and R⁵ are, independently, H, alkyl, substituted alkyl, heteroalkyl, substituted heteroalkyl, aryl, or substituted aryl.

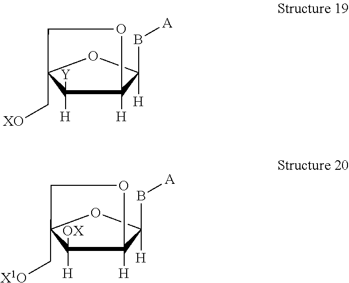

where the substituents of Structure 19 and Structure 20 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; and, Y is OH or OR² where R² is a protecting group, an alkyl, a substituted alkyl, a heteroalkyl, a substituted heteroalkyl, an aryl or a substituted aryl.

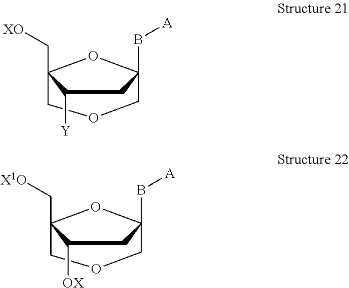

where the substituents of Structure 21 and Structure 22 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; and, Y is OH or OR² where R² is a protecting group, an alkyl, a substituted alkyl, a heteroalkyl, a substituted heteroalkyl, an aryl or a substituted aryl.

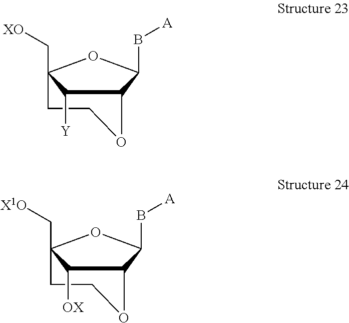

where the substituents of Structure 23 and Structure 24 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; and, Y is OH or OR² where R² is a protecting group, an alkyl, a substituted alkyl, a heteroalkyl, a substituted heteroalkyl, an aryl or a substituted aryl.

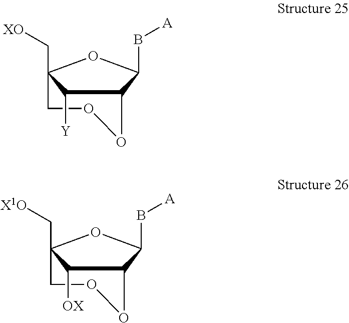

where the substituents of Structure 25 and Structure 26 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; and, Y is OH or OR² where R² is a protecting group, an alkyl, a substituted alkyl, a heteroalkyl, a substituted heteroalkyl, an aryl or a substituted aryl.

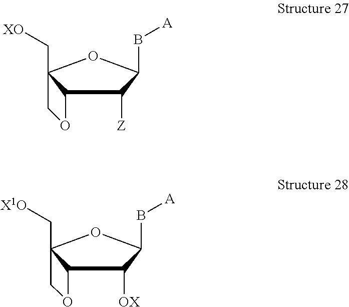

where the substituents of Structure 27 and Structure 28 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; and, Z is H, OH or OR³ where R³ is a protecting group, an alkyl, a substituted alkyl, a heteroalkyl, a substituted heteroalkyl, an aryl or a substituted aryl.

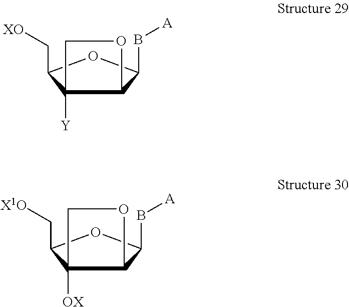

where the substituents of Structure 29 and Structure 30 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; and, Y is OH or OR² where R² is a protecting group, an alkyl, a substituted alkyl, a heteroalkyl, a substituted heteroalkyl, an aryl or a substituted aryl.

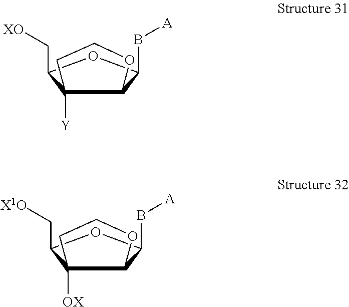

where the substituents of Structure 31 and Structure 32 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; and, Y is OH or OR² where R² is a protecting group, an alkyl, a substituted alkyl, a heteroalkyl, a substituted heteroalkyl, an aryl or a substituted aryl.

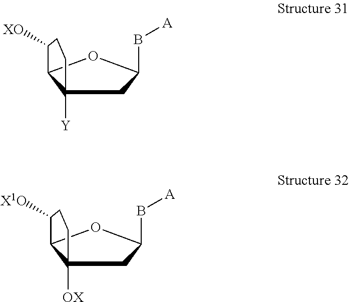

where the substituents of Structure 31 and Structure 32 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; and, Y is OH or OR² where R² is a protecting group, an alkyl, a substituted alkyl, a heteroalkyl, a substituted heteroalkyl, an aryl or a substituted aryl.

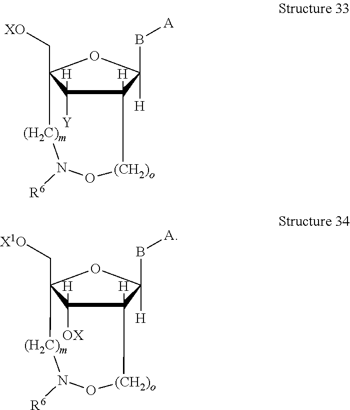

where the substituents of Structure 33 and Structure 34 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; Y is OH or OR² where R² is a protecting group, an alkyl, a substituted alkyl, a heteroalkyl, a substituted heteroalkyl, an aryl or a substituted aryl; “R⁶” is alkyl, substituted alkyl, aryl or substituted aryl; “m” and “o” and independently 0, 1 or 2.

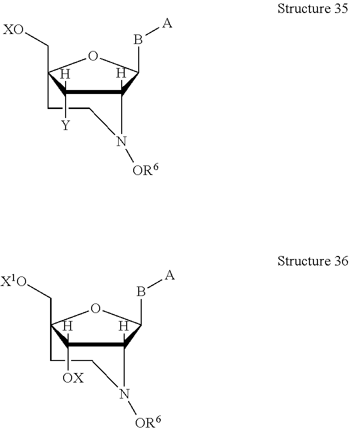

where the substituents of Structure 35 and Structure 36 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; Y is OH or OR² where R² is a protecting group, an alkyl, a substituted alkyl, a heteroalkyl, a substituted heteroalkyl, an aryl or a substituted aryl; “R⁶” is alkyl, substituted alkyl, aryl or substituted aryl.

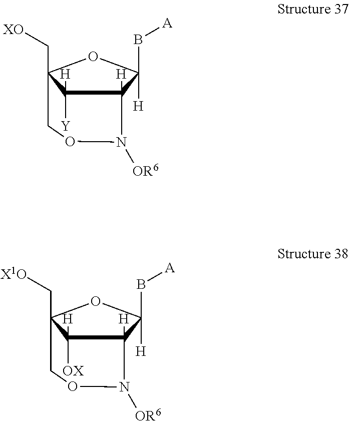

where the substituents of Structure 37 and Structure 38 are: “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); “X¹” is H, a protecting group, a solid support which optionally includes a linker between the oxygen and the solid support, a phosphorus containing moiety or salts thereof; Y is OH or OR² where R² is a protecting group, an alkyl, a substituted alkyl, a heteroalkyl, a substituted heteroalkyl, an aryl or a substituted aryl; “R⁶” is alkyl, substituted alkyl, aryl or substituted aryl.

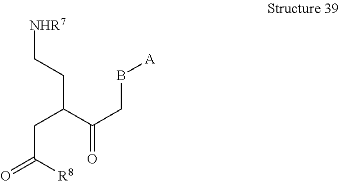

where the substituents of Structure 39 are: “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group (e.g., —C(CH₃)₃); R⁷ is H, alkyl, substituted alkyl, heteroalkyl, substituted heteroalkyl, aryl, substituted aryl or a protecting group; R⁸ is OH, a halide, OR⁹, NR¹⁰R¹¹, where R⁹ is alkyl, substituted alkyl, aryl, heteroalkyl, substituted heteroalkyl, aryl, or substituted aryl, and where R¹⁰ and R¹¹ are independently H, alkyl, substituted alkyl, aryl, heteroalkyl, substituted heteroalkyl, aryl, or substituted aryl.

Nonlimiting examples of nucleobase moieties include:

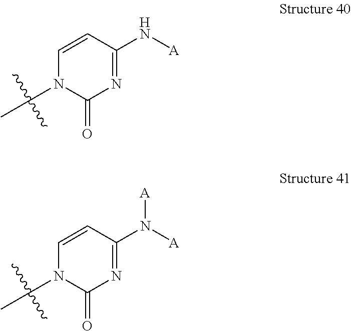

where the substituent “A” in Structure 40 and Structure 41 above is of the structure —C(O)OR¹, where R¹ is a tertiary alkyl group (e.g., —C(CH₃)₃).

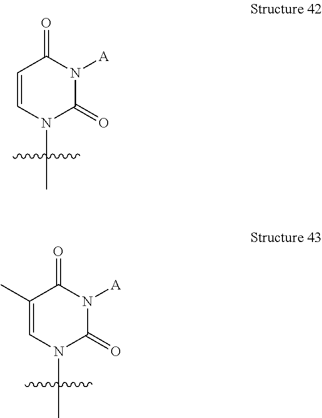

where the substituent “A” in Structure 42 and Structure 43 above is of the structure —C(O)OR¹, where R¹ is a tertiary alkyl group (e.g., —C(CH₃)₃).

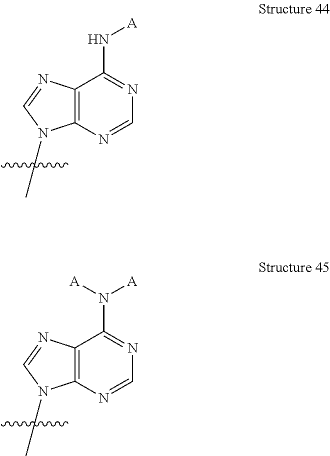

where substituent “A” of Structure 44 and Structure 45 above is of the structure —C(O)OR¹, where R¹ is a tertiary alkyl group (e.g., —C(CH₃)₃).

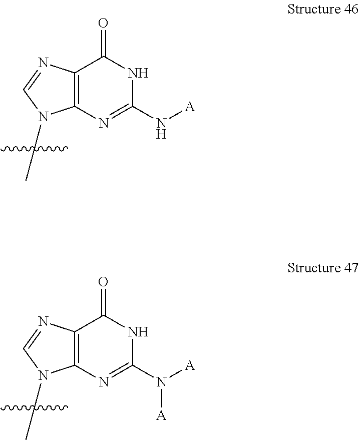

where substituent “A” of Structure 46 and Structure 47 above is of the structure —C(O)OR¹, where R¹ is a tertiary alkyl group (e.g., —C(CH₃)₃).

Nonlimiting examples of nucleobase analogue moieties include:

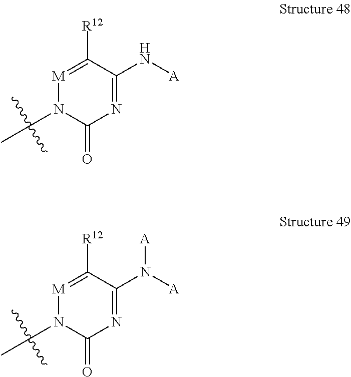

where “A” is of the structure —C(O)OR¹, where R¹ is a tertiary alkyl group (e.g., —C(CH₃)₃); where “M” is N or CR¹³, where R¹³ is H, halo, alkyl, substituted alkyl, heteroalkyl, substituted heteroalkyl, phenyl, substituted phenyl, alkenyl, alkynyl, OH, SH, or NR¹⁴R¹⁵, where R¹⁴ and R¹⁵ are, independently H or alkyl; and where R¹² is H, halo, alkyl, substituted alkyl, heteroalkyl, substituted heteroalkyl, phenyl, substituted phenyl, alkenyl, alkynyl, OH, SH, or NR¹⁴R¹⁵, where R¹⁴ and R¹⁵ are, independently H or alkyl.

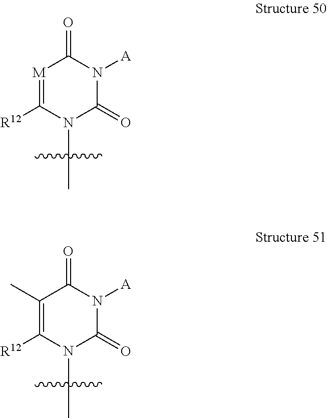

where the substituents of Structure 50 and Structure 51 above are: “A” is of the structure —C(O)OR¹, where R¹ is a tertiary alkyl group (e.g., —C(CH₃)₃); where “M” is N or CR¹³, where R¹³ is H, halo, alkyl, substituted alkyl, heteroalkyl, substituted heteroalkyl, phenyl, substituted phenyl, alkenyl, alkynyl, OH, SH, or NR¹⁴R¹⁵, where R¹⁴ and R¹⁵ are, independently H or alkyl; and where R¹² is H, halo, alkyl, substituted alkyl, heteroalkyl, substituted heteroalkyl, phenyl, substituted phenyl, alkenyl, alkynyl, OH, SH, or NR¹⁴R¹⁵, where R¹⁴ and R¹⁵ are, independently H or alkyl.

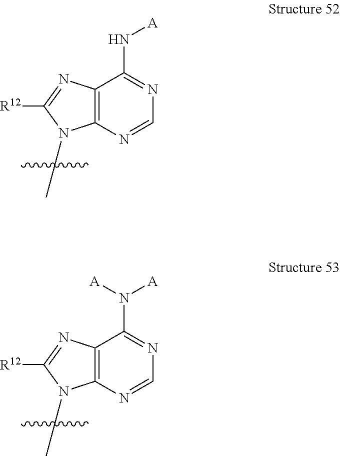

where the substituents of Structure 52 and Structure 53 above are: “A” is of the structure —C(O)OR¹, where R¹ is a tertiary alkyl group (e.g., —C(CH₃)₃); and where R¹² is H, halo, alkyl, substituted alkyl, heteroalkyl, substituted heteroalkyl, phenyl, substituted phenyl, alkenyl, alkynyl, OH, SH, or NR¹⁴R¹⁵, where R¹⁴ and R¹⁵ are, independently H or alkyl.

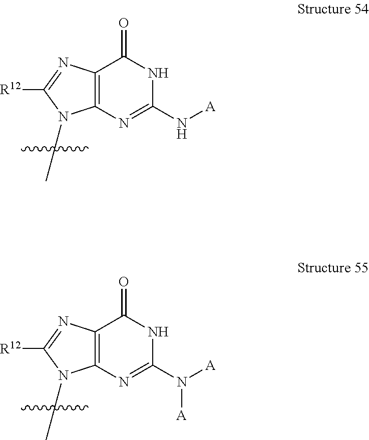

where the substituents of Structure 54 and Structure 55 above are: “A” is of the structure —C(O)OR¹, where R¹ is a tertiary alkyl group (e.g., —C(CH₃)₃); and where R¹² is H, halo, alkyl, substituted alkyl, heteroalkyl, substituted heteroalkyl, phenyl, substituted phenyl, alkenyl, alkynyl, OH, SH, or NR¹⁴R¹⁵, where R¹⁴ and R¹⁵ are, independently H or alkyl.

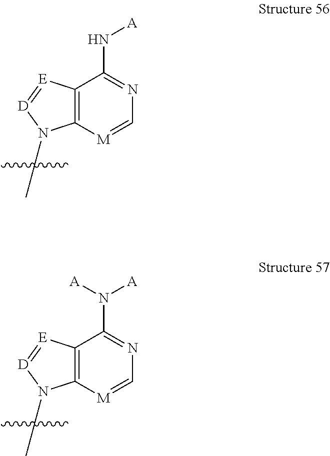

where the substituents of Structure 56 and Structure 57 above are: “A” is of the structure —C(O)OR¹, where R¹ is a tertiary alkyl group (e.g., —C(CH₃)₃); and where “M”, “D” and “E” are independently N or CR¹³, where R¹³ is H, halo, alkyl, substituted alkyl, heteroalkyl, substituted heteroalkyl, phenyl, substituted phenyl, alkenyl, alkynyl, OH, SH, or NR¹⁴R¹⁵, where R¹⁴ and R¹⁵ are, independently H or alkyl.

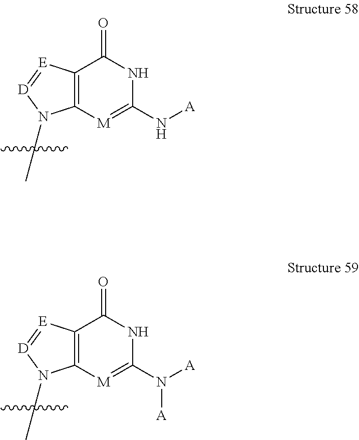

where the substituents of Structure 58 and Structure 59 are: “A” is of the structure —C(O)OR¹, where R¹ is a tertiary alkyl group (e.g., —C(CH₃)₃); and where “M”, “D” and “E” are independently N or CR¹³, where R¹³ is H, halo, alkyl, substituted alkyl, heteroalkyl, substituted heteroalkyl, phenyl, substituted phenyl, alkenyl, alkynyl, OH, SH, or NR¹⁴R¹⁵, where R¹⁴ and R¹⁵ are, independently H or alkyl.

For a discussion of nucleoside analogues, see: Merino, P. (Ed.) (2013) Chemical Synthesis of Nucleotide Analogues, Pedro Marino, Wiley Publishers; U.S. Pat. No. 7,427,672; Prakash, T. et al. J. Med. Chem. 2010, 53, 1636-1650. The preceding reference is hereby incorporated-by-reference into this document for all purposes.

The moiety “A” is of the structure —C(O)R¹ wherein R¹ is tertiary alkyl. A tertiary alkyl is one where a carbon atom is covalently bound to three groups (i.e., —CR¹⁶R¹⁷R¹⁸), where R¹⁶, R¹⁷ and R¹⁸ are independently selected from alkyl, substituted alkyl, heteroalkyl and substituted heteroalkyl. Typically, the substituents R¹⁶, R¹⁷ and R¹⁸ terminate in a CH₂ or CH₃ that is bound directly to the central carbon atom (e.g., —C(CH₃)₂(CH₂CH₃). Nonlimiting examples of tertiary alkyl groups include: —C(CH₃)₃; —C(CH₃)₂(CH₂CH₃); —C(CH₃)(CH₂CH₃)(CH₂CH₂CH₃); —C(R⁹)(R²⁰)—Linker-Label; and —C(R¹⁹)(R²⁰)—Linker-[Solid Support], wherein R¹⁹ and R²⁰ are independently selected from —CH₃, —CH₂CH₃, —CH₂CH₂CH₃, and CH(CH₃)_{2.}

Nonlimiting examples of —C(R⁹)(R²)—Linker-Label include:

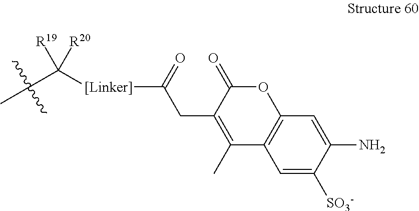

wherein the substituents of Structure 60 above are: R¹⁹ and R²⁰ are independently selected from —CH₃, —CH₂CH₃, —CH₂CH₂CH₃, and CH(CH₃)₂.

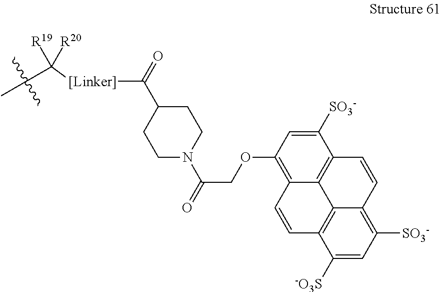

wherein the substituents of Structure 61 above are: R¹⁹ and R²⁰ are independently selected from —CH₃, —CH₂CH₃, —CH₂CH₂CH₃, and CH(CH₃)₂.

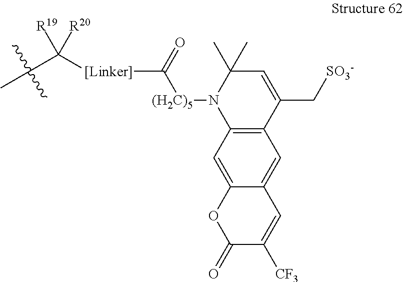

wherein the substituents of Structure 62 above are: R¹⁹ and R²⁰ are independently selected from —CH₃, —CH₂CH₃, —CH₂CH₂CH₃, and CH(CH₃)₂.

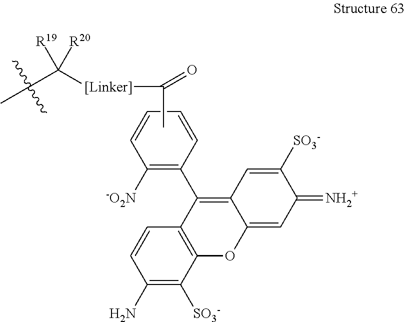

wherein the substituents of Structure 63 above are: R¹⁹ and R²⁰ are independently selected from —CH₃, —CH₂CH₃, —CH₂CH₂CH₃, and CH(CH₃)₂.

Nonlimiting examples of —C(R′)(R²⁰)—Linker-[Solid Support], include:

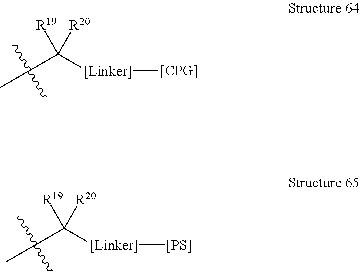

wherein the substituents of Structure 64 and Structure 65 above are: R¹⁹ and R²⁰ are independently selected from —CH₃, —CH₂CH₃, —CH₂CH₂CH₃, and CH(CH₃)₂; CPG is controlled pore glass; and, PS is polystyrene.

In one case, when SM is of the structure

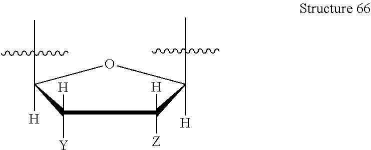

where the substituents of Structure 66 above are: Y is —OP(O-CNE)ONR⁵¹R⁵² or —OP(O)(OH)H or salts thereof, where R⁵¹ and R⁵² are independently selected from alkyl, substituted alkyl, aryl or substituted aryl or R⁵¹ and R⁵² together form a heterocycle (e.g., pyrrolidine), then X is an acid labile protecting group or a solid support, Z is H or OR⁵³, and R⁵³ is a hydroxyl protecting group.

In another case, when SM is of the structure

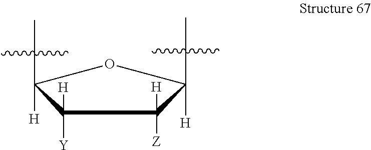

where the substituents of Structure 67 above are: X is —P(O-CNE)(NR⁵¹R⁵²) or —P(O)(OR⁵³)H or salts thereof, where R⁵¹ and R⁵² are independently selected from alkyl, substituted alkyl, aryl or substituted aryl or R⁵¹ and R⁵² together form a heterocycle (e.g., pyrrolidine), and where R⁵³ is alkyl, substituted alkyl, aryl or substituted alkyl, then Y is an acid labile hydroxyl protecting group or a solid support and Z is H.

In another case, when SM is of the structure

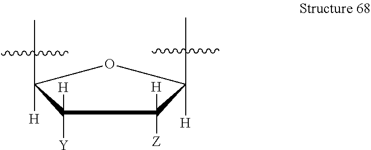

where the substituents of Structure 68 above are: X is —P(O)(OR⁵³)H or —P(O)(OH)O[P(O)(O⁻)(O⁻)]ₙH or salts thereof, where R⁵³ is alkyl, substituted alkyl, aryl or substituted aryl, wherein n=0, 1 or 2, then Y is OH or OR⁵⁴ wherein R⁵⁴ is a thermolabile hydroxyl protecting group, and Z is H, —OH, or OR⁵⁴.

Nonlimiting examples of compounds of the present invention include the following:

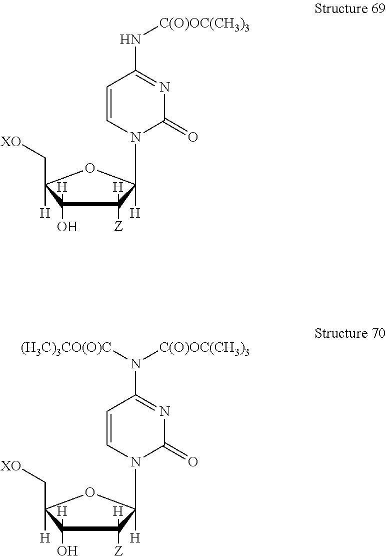

where the substituents of Structure 69 and Structure 70 above are: “X” is —P(O)(OH)₂, —P(O)(OH)OP(O)(OH)₂, —P(O)(OH)OP(O)(OH)OP(O)(OH)₂ or salts thereof, and where “Z” is —H or —OH.

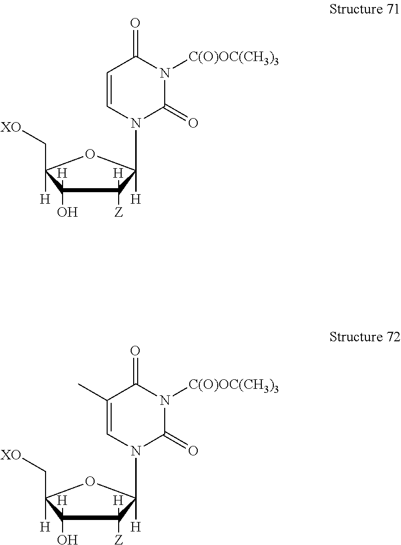

where the substituents of Structure 71 and 72 above are: “X” is —P(O)(OH)₂, —P(O)(OH)OP(O)(OH)₂, —P(O)(OH)OP(O)(OH)OP(O)(OH)₂ or salts thereof, and where “Z” is —H or —OH.

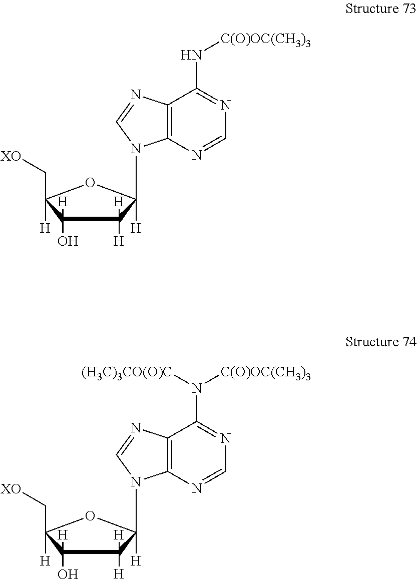

where the substituents of Structure 73 and Structure 74 above are: “X” is —P(O)(OH)₂, —P(O)(OH)OP(O)(OH)₂, —P(O)(OH)OP(O)(OH)OP(O)(OH)₂ or salts thereof, and where “Z” is —H or —OH.

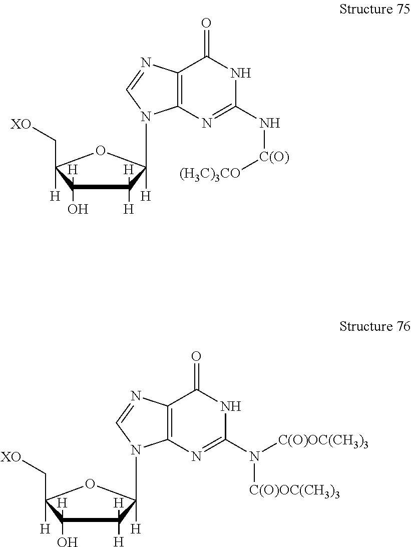

where the substituents of Structure 75 and Structure 76 above are: “X” is —P(O)(OH)₂, —P(O)(OH)OP(O)(OH)₂, —P(O)(OH)OP(O)(OH)OP(O)(OH)₂ or salts thereof, and where “Z” is —H or —OH.

The present invention is further directed to oligonucleotides, and salts thereof, including one or more nucleotides or nucleotide analogues of the structure —O—CH₂—SM(—O—)B-A, where “SM” is a sugar moiety or an analogue of a sugar moiety; “B” is a nucleobase moiety or an analogue of a nucleobase moiety; “A” is one or more moieties attached to one or more nitrogen atoms on or within the base moiety and is of the structure —C(O)OR⁶⁰, where R⁶⁰ is a tertiary alkyl group.

For a discussion of oligonucleotide synthesis, see: Ellington, A. and Pollard, J. D. 2001. Introduction to the Synthesis and Purification of Oligonucleotides. Current Protocols in Nucleic Acid Chemistry. 00:A.3C.1-A.3C.22; Beaucage, S. L. and Reese, C. B. 2009. Recent Advances in the Chemical Synthesis of RNA. Current Protocols in Nucleic Acid Chemistry. 38:2.16.1-2.16.31; Tsukamoto, M. and Hayakawa, Y.2005. “Strategies useful for the Chemical Synthesis of Oligonucleotides and Related Compounds.” Frontiers in Organic Chemistry, Bentham Science Publishers, Vol. 1. The preceding references are hereby incorporated-by-reference into this document for all purposes.

In one aspect, the oligonucleotide is of the following structure:

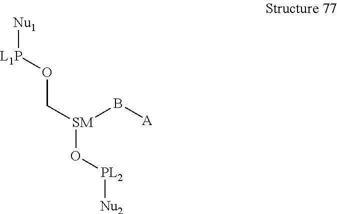

where the substituents of Structure 77 above are: PL₁ and PL₂ are, independently, either H or —P(O)(OH)O— or an analogue thereof, and Nu₁ and Nu₂ are, independently, no substituent, a nucleoside or nucleoside analogue, or an oligonucleotide; “SM” is a sugar moiety or an analogue of a sugar moiety; “B” is a nucleobase moiety or an analogue of a nucleobase moiety; “A” is one or more moieties attached to one or more nitrogen atoms on or within the base moiety and is of the structure —C(O)OR⁶, where R⁶⁰ is a tertiary alkyl group.

In another aspect, the oligonucleotide is of one of the following structures (or salts thereof):

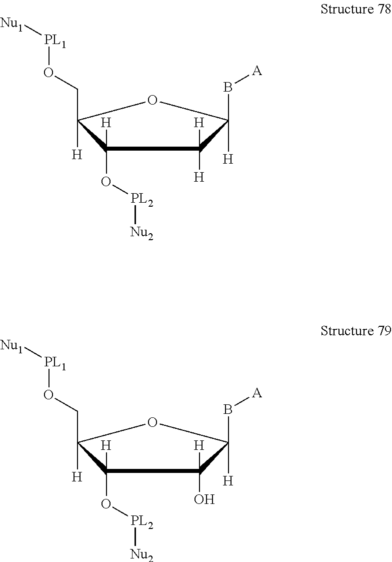

where the substituents of Structure 78 and Structure 79 above are: PL₁ and PL₂ are, independently, either H or —P(O)(OH)O— or an analogue thereof, and Nu₁ and Nu₂ are, independently, no substituent, a nucleoside or nucleoside analogue, or an oligonucleotide; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group.

In another aspect, the oligonucleotide is of one of the following structures (or salts thereof):

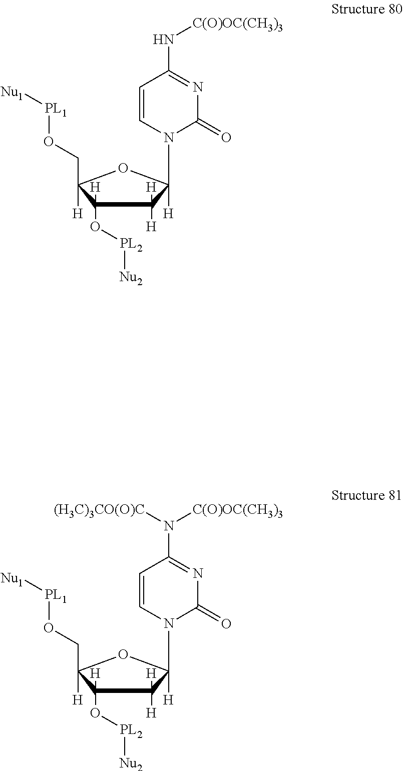

where the substituents of Structure 80 and Structure 81 above are: PL₁ and PL₂ are, independently, either H or —P(O)(OH)O— or an analogue thereof, and Nu₁ and Nu₂ are, independently, no substituent, a nucleoside or nucleoside analogue, or an oligonucleotide (or salts thereof);

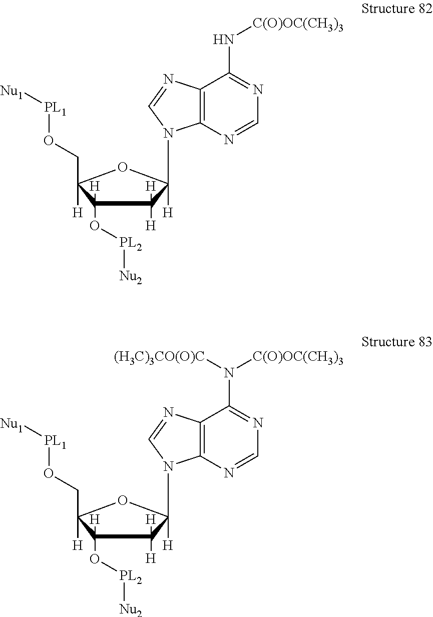

where the substituents of Structure 82 and Structure 83 above are: PL₁ and PL₂ are, independently, either H or —P(O)(OH)O— or an analogue thereof, and Nu₁ and Nu₂ are, independently, no substituent, a nucleoside or nucleoside analogue, or an oligonucleotide (or salts thereof);

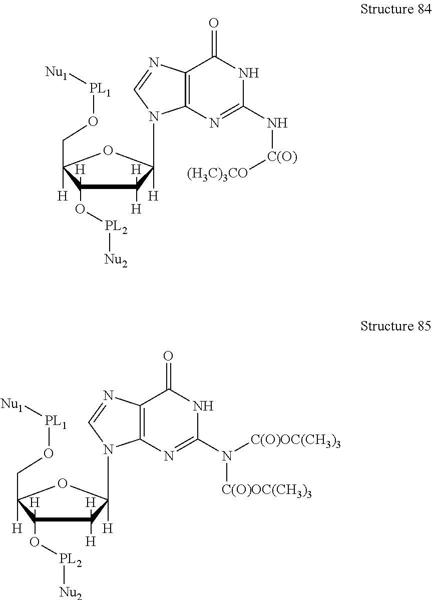

where the substituents of Structure 84 and Structure 85 above are: PL₁ and PL₂ are, independently, either H or —P(O)(OH)O— or an analogue thereof, and Nu₁ and Nu₂ are, independently, no substituent, a nucleoside or nucleoside analogue, or an oligonucleotide (or salts thereof);

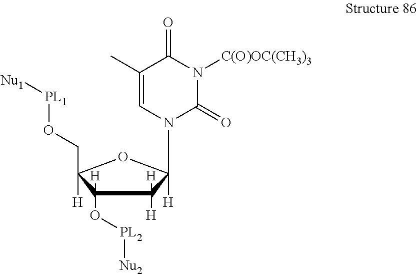

where the substituents of Structure 86 above are: PL₁ and PL₂ are, independently, either H or —P(O)(OH)O— or an analogue thereof, and Nu₁ and Nu₂ are, independently, no substituent, a nucleoside or nucleoside analogue, or an oligonucleotide (or salts thereof);

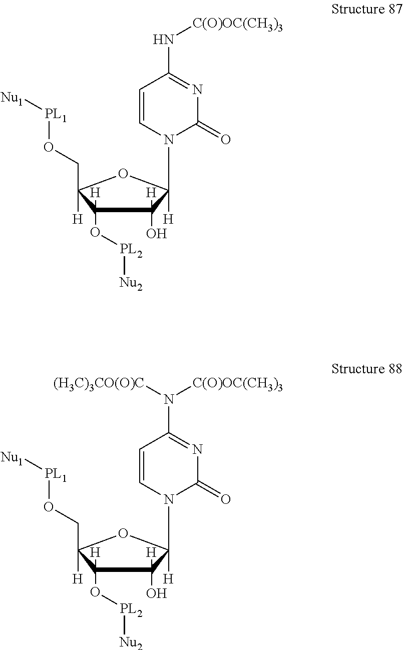

where the substituents of Structure 87 and Structure 88 above are: PL₁ and PL₂ are, independently, either H or —P(O)(OH)O— or an analogue thereof, and Nu₁ and Nu₂ are, independently, no substituent, a nucleoside or nucleoside analogue, or an oligonucleotide (or salts thereof).

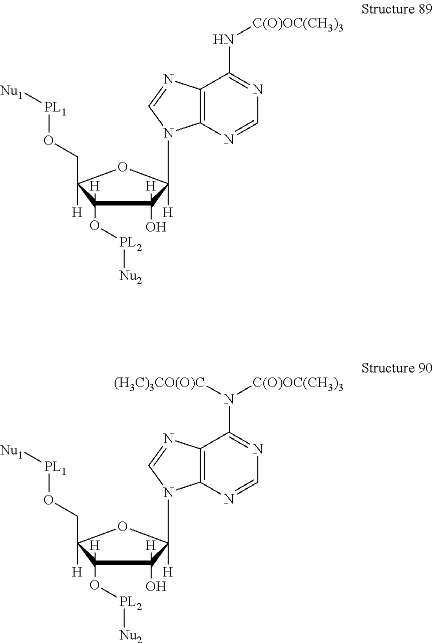

where the substituents of Structure 89 and Structure 90 above are: PL₁ and PL₂ are, independently, either H or —P(O)(OH)O— or an analogue thereof, and Nu₁ and Nu₂ are, independently, no substituent, a nucleoside or nucleoside analogue, or an oligonucleotide (or salts thereof).

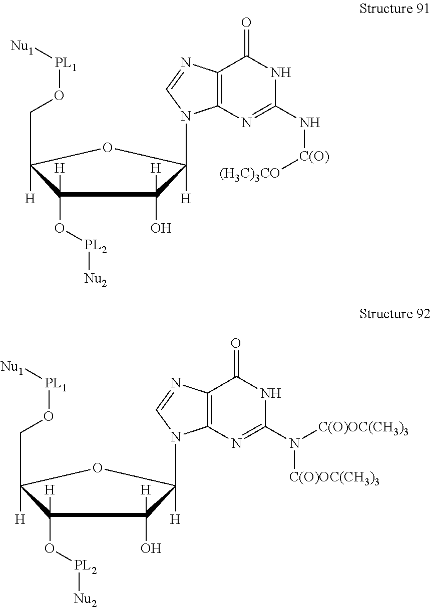

where the substituents of Structure 91 and Structure 92 above are: PL₁ and PL₂ are, independently, either H or —P(O)(OH)O— or an analogue thereof, and Nu₁ and Nu₂ are, independently, no substituent, a nucleoside or nucleoside analogue, or an oligonucleotide (or salts thereof).

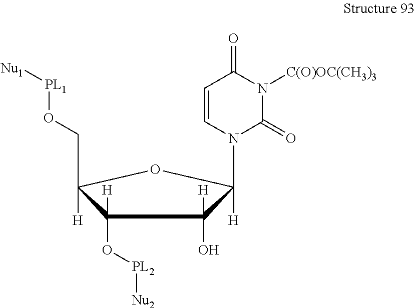

where the substituents of Structure 93 above are: PL₁ and PL₂ are, independently, either H or —P(O)(OH)O— or an analogue thereof, and Nu₁ and Nu₂ are, independently, no substituent, a nucleoside or nucleoside analogue, or an oligonucleotide (or salts thereof).

In another aspect, the oligonucleotides include two or more, three or more, four or more, five or more, or six or more nucleotides or nucleotide analogues of the structures shown above.

The present invention is further directed to certain therapeutic nucleotides, nucleotide analogues, nucleosides and nucleoside analogues. A therapeutic nucleotide, nucleotide analogue, nucleoside or nucleoside analogue is one that can be used to treat a disease (e.g., HCV), where the compound includes a nucleotide, nucleotide analogue, nucleoside or nucleoside analogue and one or more thermally labile protecting groups, where at least one of the thermally labile protecting groups is of the structure —C(O)OR⁶⁰, and where R⁶⁰ is a tertiary alkyl group (e.g., —C(CH₃)₃).

For a discussion of therapeutic nucleotides, nucleotide analogues, nucleosides and nucleoside analogues, see: Lars Petter Jordheim et al. Nature Reviews Drug Discovery, 447-464 (2013); Squires, K. Antivir. Ther. 2001; 6 Suppl 3:1-14; U.S. Pat. No. 8,664,386; U.S. Pat. No. 8,658,617; U.S. Pat. No. 8,642,756; U.S. Pat. No. 8,633,309; U.S. Pat. No. 8,629,263; U.S. Pat. No. 8,618,076; U.S. Pat. No. 8,580,765; U.S. Pat. No. 8,569,478; U.S. Pat. No. 8,563,530; U.S. Pat. No. 8,551,973. The preceding references are hereby incorporated-by-reference into this document for all purposes.

In one aspect, the therapeutic nucleotide, nucleotide analogue, nucleoside or nucleoside analogue is of one of the following structures:

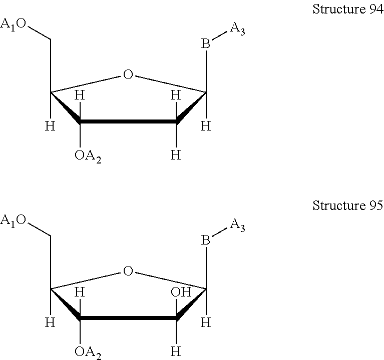

where the substituents of Structure 94 and Structure 95 above are: A₁, A₂ and A₃ are independently H or a thermally labile protecting group, and where at least one of the thermally labile protecting groups is of the structure —C(O)OR⁶, and where R⁶⁰ is a tertiary alkyl group (e.g., —C(CH₃)₃), and B is a nucleobase or nucleobase analogue;

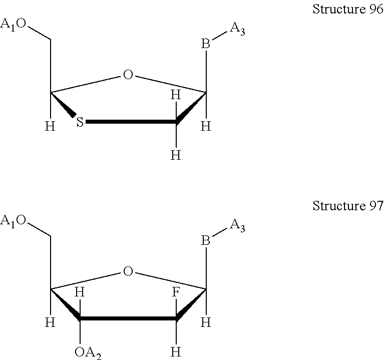

where the substituents of Structure 96 and Structure 97 above are: A₁, A₂ and A₃ are independently H or a thermally labile protecting group, and where at least one of the thermally labile protecting groups is of the structure —C(O)OR⁶⁰, and where R⁶⁰ is a tertiary alkyl group (e.g., —C(CH₃)₃), and B is a nucleobase or nucleobase analogue;

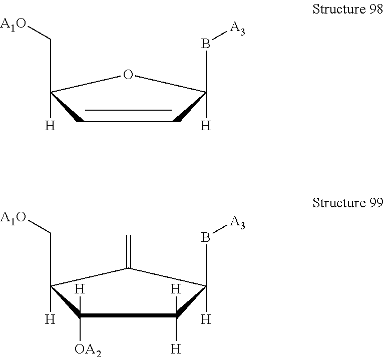

where the substituents of Structure 98 and Structure 99 above are: A₁, A₂ and A₃ are independently H or a thermally labile protecting group, and where at least one of the thermally labile protecting groups is of the structure —C(O)OR⁶⁰, and where R⁶⁰ is a tertiary alkyl group (e.g., —C(CH₃)₃), and B is a nucleobase or nucleobase analogue;

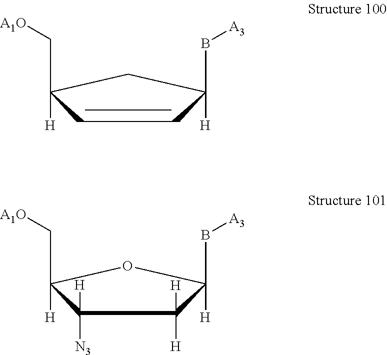

where the substituents of Structure 100 and Structure 101 above are: A₁ and A₃ are independently H or a thermally labile protecting group, and where at least one of the thermally labile protecting groups is of the structure —C(O)OR⁶⁰, and where R⁶⁰ is a tertiary alkyl group (e.g., —C(CH₃)₃), and B is a nucleobase or nucleobase analogue;

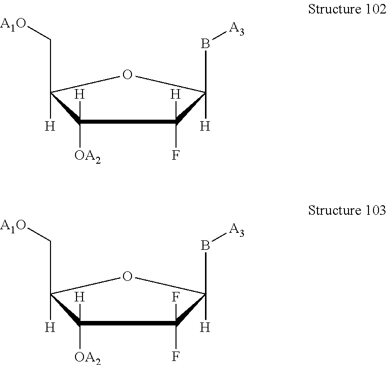

where the substituents of Structure 102 and Structure 103 above are: A₁, A₂ and A₃ are independently H or a thermally labile protecting group, and where at least one of the thermally labile protecting groups is of the structure —C(O)OR⁶⁰, and where R⁶⁰ is a tertiary alkyl group (e.g., —C(CH₃)₃), and B is a nucleobase or nucleobase analogue;

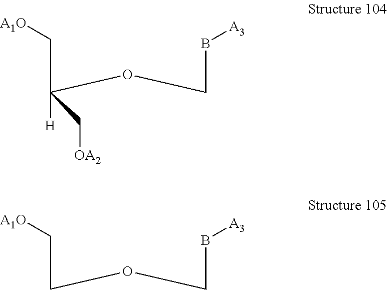

where the substituents of Structure 104 and Structure 105 above are: A₁, A₂ and A₃ are independently H or a thermally labile protecting group, and where at least one of the thermally labile protecting groups is of the structure —C(O)OR⁶⁰, and where R⁶⁰ is a tertiary alkyl group (e.g., —C(CH₃)₃), and B is a nucleobase or nucleobase analogue;

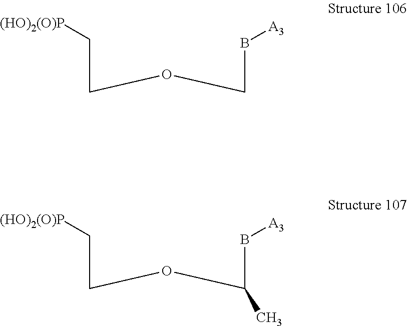

where the substituents of Structure 106 and Structure 107 above are: A₃ is H or a thermally labile protecting group, and where at least one of the thermally labile protecting groups is of the structure —C(O)OR⁶⁰, and where R⁶⁰ is a tertiary alkyl group (e.g., —C(CH₃)₃), and B is a nucleobase or nucleobase analogue;

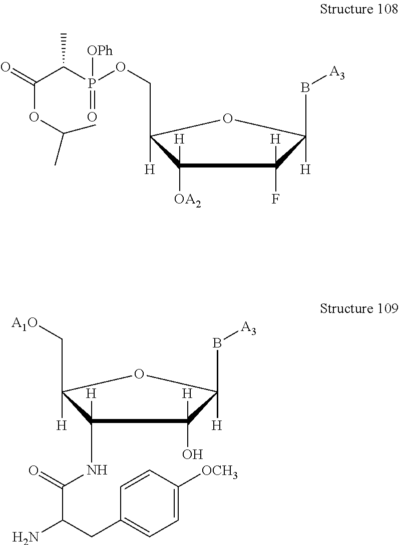

where the substituents of Structure 108 and Structure 109 above are: A₁, A₂ and A₃ are independently H or a thermally labile protecting group, and where at least one of the thermally labile protecting groups is of the structure —C(O)OR⁶⁰, and where R⁶⁰ is a tertiary alkyl group (e.g., —C(CH₃)₃), and B is a nucleobase or nucleobase analogue.

In another aspect, the therapeutic nucleotide, nucleotide analogue, nucleoside or nucleoside analogue is of one of the following structures:

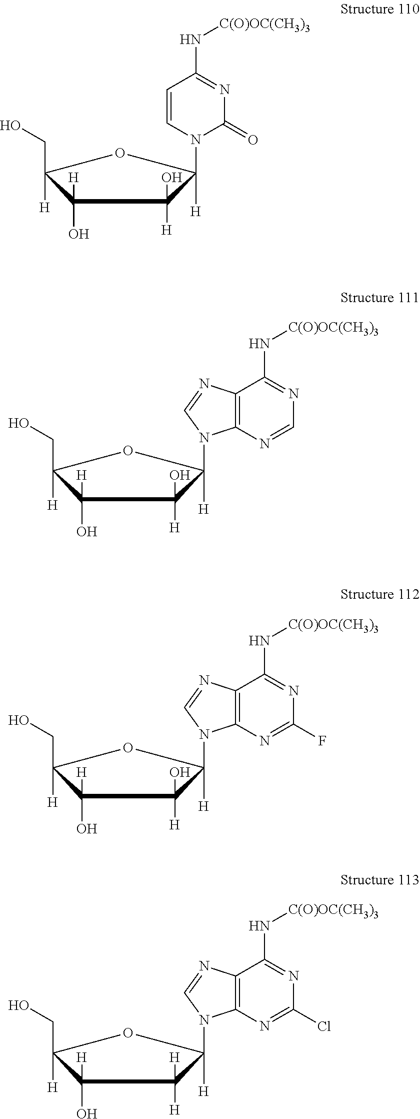

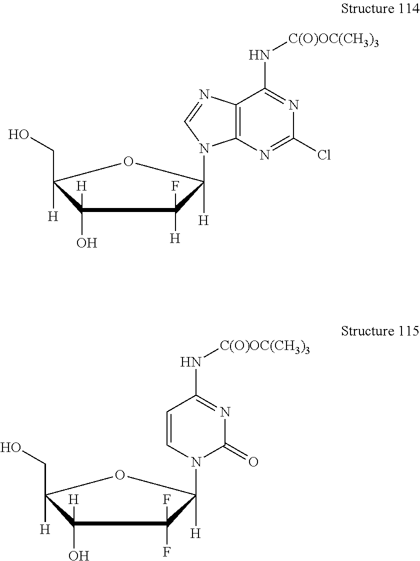

The present invention is further directed to therapeutic oligonucleotides (or salts thereof). A therapeutic oligonucleotide is one that can be used to treat a disease (e.g., CMV), where the compound includes an oligonucleotide (e.g., Fomivirsen, Mipomersen) containing one or more thermally labile protecting groups. At least one of the thermally labile protecting groups is of the structure —C(O)OR⁶⁰, and where R⁶⁰ is a tertiary alkyl group (e.g., —C(CH₃)₃).

The therapeutic oligonucleotide is typically of the following structure (or salts thereof):

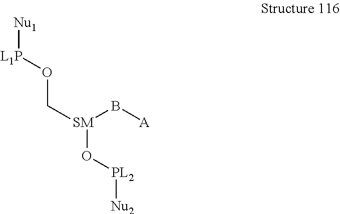

where the substituents of Structure 116 above are: PL₁ and PL₂ are, independently, either H or —P(O)(OH)O— or an analogue thereof, and Nu₁ and Nu₂ are, independently, no substituent, a nucleoside or nucleoside analogue, or an oligonucleotide (or salts thereof). “SM” is a sugar moiety or an analogue of a sugar moiety; “B” is a nucleobase moiety or an analogue of a nucleobase moiety; “A” is one or more moieties attached to one or more nitrogen atoms on or within the base moiety and is of the structure —C(O)OR⁶⁰, where R⁶⁰ is a tertiary alkyl group.

For a discussion of therapeutic oligonucleotides, see: Yogesh S. Sanghvi Current Protocols in Nucleic Acid Chemistry, 4.1.1-4.1.22, September 2011; Goodchild, J. Methods Mol. Biol. 2011; 764:1-15; U.S. Pat. No. 8,697,675. The preceding references are hereby incorporated-by-reference into this document for all purposes.

In another aspect, the present invention is directed to an oligonucleotide-label conjugate (or salts thereof). The oligonucleotide-label conjugate includes one or more nucleotides or nucleotide analogues of the following structure:

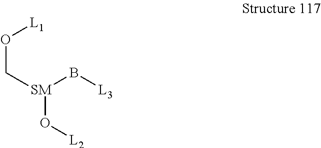

where the substituents of Structure 117 above are: L₁ and L₂ are independently H, a nucleotide, a nucleotide analogue, and a label, where there may be a linking group connecting the label to its position on the nucleotide or nucleotide analogue; L₃ is H, —C(O)OR⁶⁰ where R⁶⁰ is a tertiary alkyl (e.g., —C(CH₃)₃), or a label, where there may be a linking group connecting the label to its position on the nucleotide or nucleotide analogue. If the label is not L₁, L₂ or L₃, it is attached to another nucleotide of the oligonucleotide. “SM” is a sugar moiety or an analogue of a sugar moiety; “B” is a nucleobase moiety or an analogue of a nucleobase moiety.

For a discussion of oligonucleotide-label conjugates, see: U.S. Pat. No. 5,583,236; U.S. Pat. No. 8,530,634; Durrant, Ian et al. Methods in Molecular Biology, Vol. 31 (1994), 163-175. The preceding references are hereby incorporated-by-reference into this document for all purposes.

In another aspect, the present invention is directed to a method of synthesizing an oligonucleotide (or salts thereof). The method comprises the following steps:

1) Coupling a compound to a solid support, either directly or through a linker, where the compound is of one of the following structures:

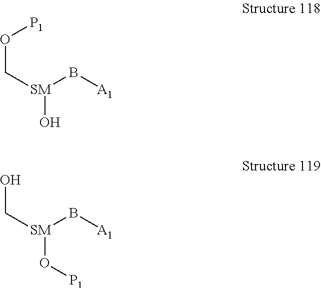

where the substituents of Structure 118 and Structure 119 above are: “P₁” is a protecting group (e.g., DMT), “SM” is a sugar moiety or an analogue of a sugar moiety, “B” is a nucleobase or nucleobase analogue, and “A₁” is H or —C(O)OR⁶⁰, where R⁶⁰ is a tertiary alkyl (e.g., —C(O)OC(CH₃)₃) to provide a solid support compound of one of the following structures:

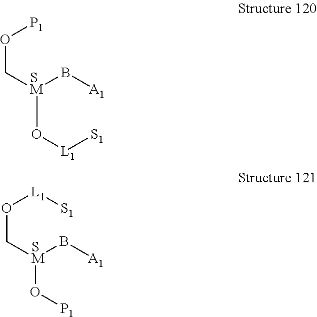

where the substituents of Structure 120 and Structure 121 above are: L₁ is a linker or no chemical entity, and S₁ is a solid support; “P₁” is a protecting group (e.g., DMT), “B” is a nucleobase or nucleobase analogue, “SM” is a sugar moiety or an analogue of a sugar moiety, and “A₁” is H or —C(O)OR⁶⁰, where R⁶⁰ is a tertiary alkyl (e.g., —C(O)OC(CH₃)₃);

2) Deprotecting the solid support compound to provide a deprotected compound of one of the following structures:

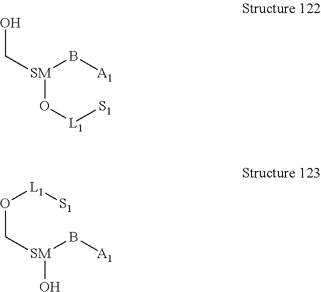

where the substituents of Structure 122 and Structure 123 above are: L₁ is a linker or no chemical entity, and S is a solid support; “B” is a nucleobase or nucleobase analogue, “SM” is a sugar moiety or an analogue of a sugar moiety, and “A₁” is H or —C(O)OR⁶⁰, where R⁶⁰ is a tertiary alkyl (e.g., —C(O)OC(CH₃)₃);

3) Reacting the deprotected compound with a compound including a moiety comprising a phosphorus atom, wherein the compound is of one of the following structures:

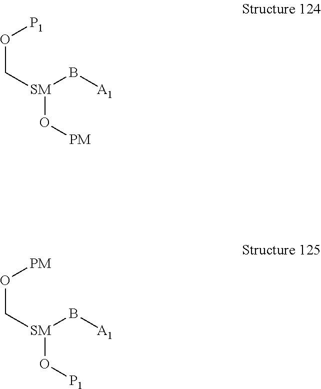

where the substituents of Structure 124 and Structure 125 above are: “PM” is a phosphorus containing moiety, “P₁” is a protecting group (e.g., DMT); “B” is a nucleobase or nucleobase analogue; “SM” is a sugar moiety or an analogue of a sugar moiety; and “A₁” is H or —C(O)OR⁶⁰, where R⁶⁰ is a tertiary alkyl (e.g., —C(O)OC(CH₃)₃), to provide a dinucleotide of one of the following structures;

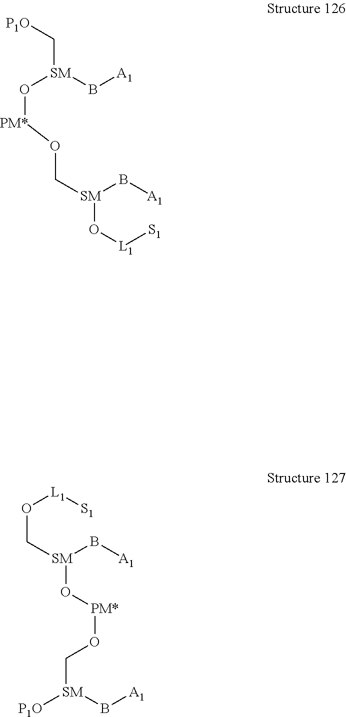

where the substituents of Structure 126 and Structure 127 above are: “PM*” is the phosphorus containing moiety after the reaction, L₁ is a linker or no chemical entity, S₁ is a solid support, “P₁” is a protecting group (e.g., DMT), “B” is a nucleobase or nucleobase analogue, “SM” is a sugar moiety or an analogue of a sugar moiety, and “A₁” is H or —C(O)OR⁶⁰, where R⁶ is a tertiary alkyl (e.g., —C(O)OC(CH₃)₃);

4) Optionally, chemically modifying the phosphorus containing moiety to provide a modified dimer of one of the following structures:

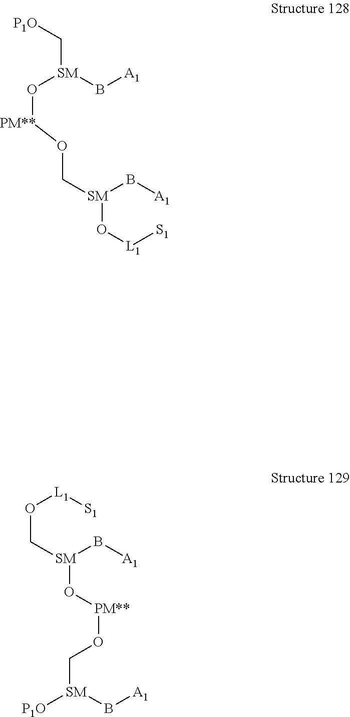

where the substituents of Structure 128 and Structure 129 above are: “PM**” is a chemically modified phosphorus containing moiety, L₁ is a linker or no chemical entity, S₁ is a solid support, “P₁” is a protecting group (e.g., DMT), “B” is a nucleobase or nucleobase analogue, “SM” is a sugar moiety or an analogue of a sugar moiety, and “A₁” is H or —C(O)OR⁶⁰, where R⁶⁰ is a tertiary alkyl (e.g., —C(O)OC(CH₃)₃);

5) Optionally, deprotecting the dimer or modified dimer to provide a deprotected dimer or modified dimer of one of the following structures:

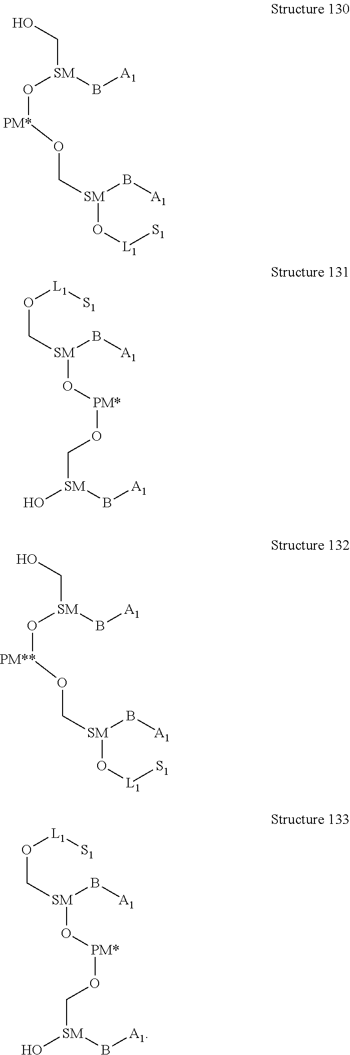

where the substituents of Structure 130, Structure 131, Structure 132 and Structure 133 above are: “PM*” is the phosphorus containing moiety after the reaction to provide a dimer, “PM**” is a chemically modified phosphorus containing moiety, L₁ is a linker or no chemical entity, S₁ is a solid support, “B” is a nucleobase or nucleobase analogue, “SM” is a sugar moiety or an analogue of a sugar moiety, and “A₁” is H or —C(O)OR⁶⁰, where R⁶⁰ is a tertiary alkyl (e.g., —C(O)OC(CH₃)₃);

6) Optionally, repeating steps “3” and “4” to provide an oligomer or modified oligomer of one of the following structures:

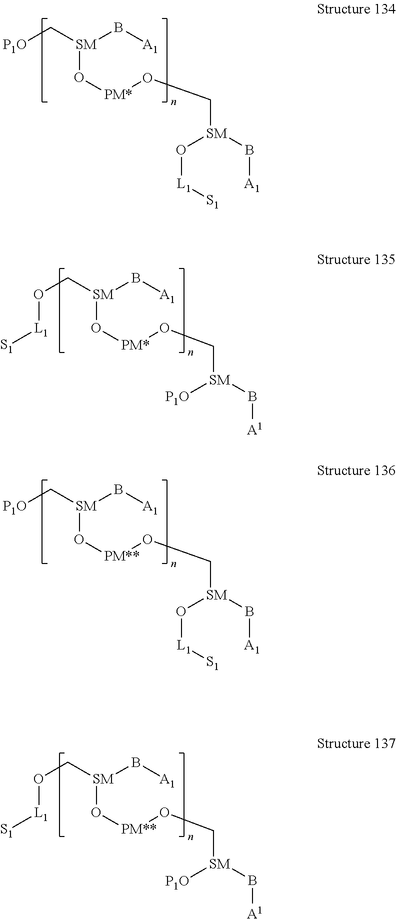

where the substituents of Structure 134, Structure 135, Structure 136 and Structure 137 above are: “P₁” is a protecting group (e.g., DMT), “PM*” is the phosphorus containing moiety after the reaction to provide an oligomer, “PM**” is a chemically modified phosphorus containing moiety, “L₁” is a linker or no chemical entity, “S₁” is a solid support, “B” is a nucleobase or nucleobase analogue, “SM” is a sugar moiety or an analogue of a sugar moiety, and “A₁” is H or —C(O)OR⁶⁰, where R⁶⁰ is a tertiary alkyl (e.g., —C(O)OC(CH₃)₃); “n” is an integer ranging from 1 to 200 (e.g., 1 to 25, 1 to 50, 1 to 75, 1 to 100, etc.);

7) Deprotecting the dimer, modified dimer, oligonucleotide or modified oligonucleotide, removing it from the solid support, and chemically modifying the PM* or PM** moiety to provide a compound of the following structure:

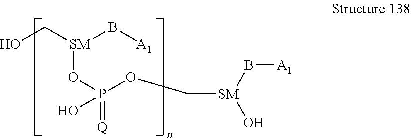

where the substituents of Structure 138 above are: “Q” is O or S, and where “n” is an integer ranging from 1 to 200 (e.g., 1 to 25, 1 to 50, 1 to 75, 1 to 100, etc.), where at least one “A₁” is —C(O)OR⁶⁰, where R⁶⁰ is tertiary alkyl (e.g., —C(CH₃)₃), “B” is a nucleobase or nucleobase analogue, and “SM” is a sugar moiety or an analogue of a sugar moiety.

In one case, the compound coupled to the solid support in step “1” of the above recited method is one of the following structures:

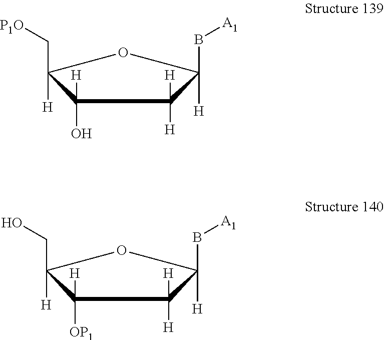

where the substituents of Structure 139 and Structure 140 above are: “P₁” is a protecting group (e.g., DMT), “B” is a nucleobase or nucleobase analogue, and “A₁” is —H or —C(O)OR⁴, where R⁴ is tertiary alkyl (e.g., —C(O)OC(CH₃)₃).

In another case, the compound coupled to the solid support in step “1” of the above recited method is one of the following structures:

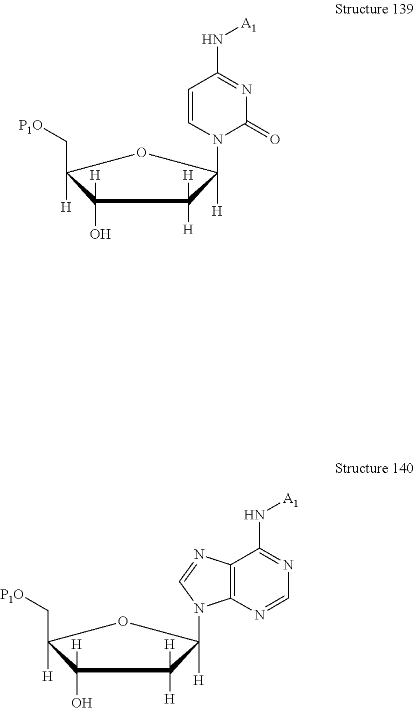

where the substituents of Structure 139 and Structure 140 above are: “P₁” is a protecting group (e.g., DMT), and “A₁” is —H or —C(O)OR⁴, where R⁴ is tertiary alkyl (e.g., —C(O)OC(CH₃)₃); or,

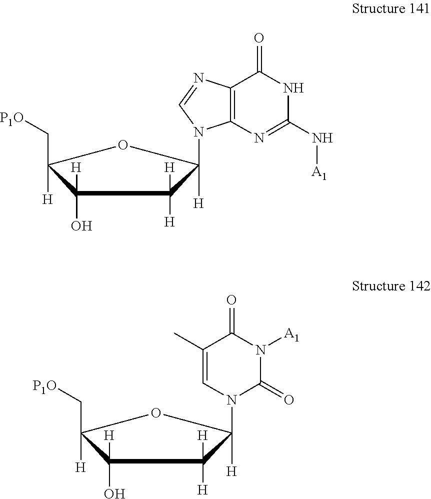

where the substituents of Structure 141 and Structure 142 above are: “P₁” is a protecting group (e.g., DMT), and “A₁” is —H or —C(O)OR₄, where R₄ is tertiary alkyl (e.g., —C(O)OC(CH₃)₃).

In one case, the deprotected structure in step “2” of the method recited above is one of the following structures:

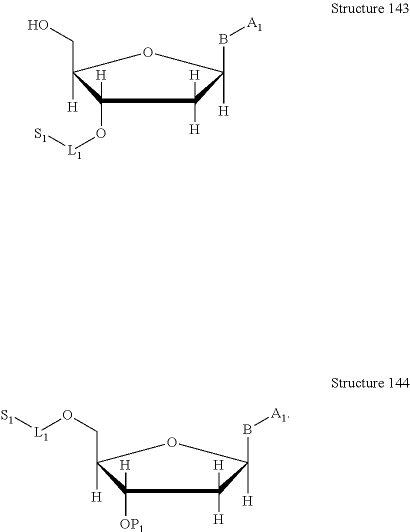

where the substituents of Structure 143 and Structure 144 above are: “B” is a nucleobase or nucleobase analogue, “A₁” is —H or —C(O)OR⁴, where R⁴ is tertiary alkyl (e.g., —C(O)OC(CH₃)₃), L₁ is a linker or no chemical entity, and S₁ is a solid support.

In one case, the deprotected structure in step “2” of the method recited above is one of the following structures:

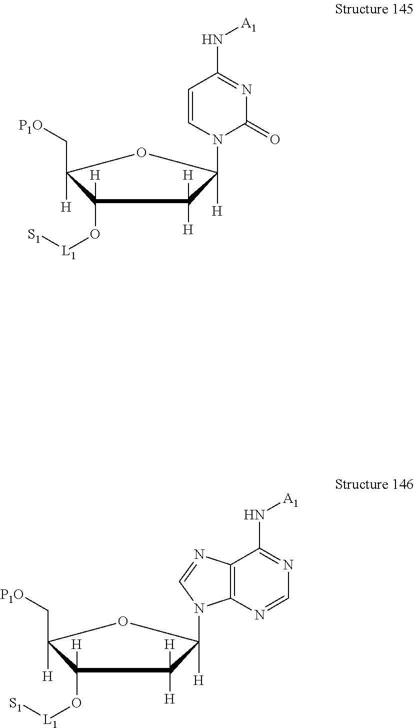

where the substituents of Structure 145 and Structure 146 above are: “P₁” is a protecting group (e.g., DMT), “A₁” is —H or —C(O)OR⁴, where R⁴ is tertiary alkyl (e.g., —C(O)OC(CH₃)₃), and where L₁ is a linker or no chemical moiety, and S₁ is a solid support; or

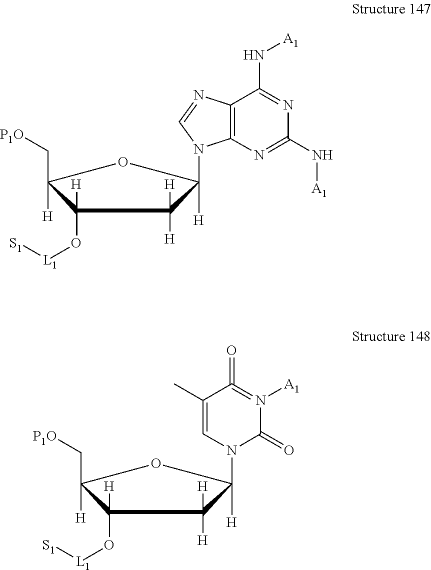

where the substituents of Structure 147 and Structure 148 above are: “P₁” is a protecting group (e.g., DMT), “A₁” is —H or —C(O)OR⁴, where R⁴ is tertiary alkyl (e.g., —C(O)OC(CH₃)₃), and where L₁ is a linker or no chemical moiety, and S₁ is a solid support.

In one case, the compound including a moiety comprising a phosphorus atom in step “3” of the method recited above is of one of the following structures:

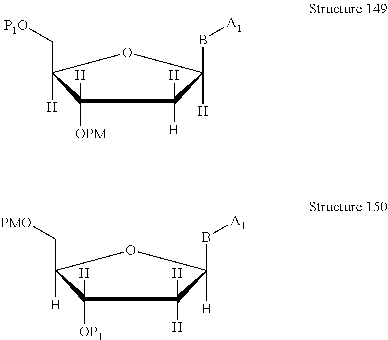

where the substituents of Structure 149 and Structure 150 above are: “P₁” is a protecting group (e.g., DMT), where “As” is —H or —C(O)OR⁴, where R⁴ is tertiary alkyl (e.g., —C(O)OC(CH₃)₃), where “B” is a nucleobase or nucleobase analogue, and “PM” is a phosphorus containing moiety selected from one of the following moieties:

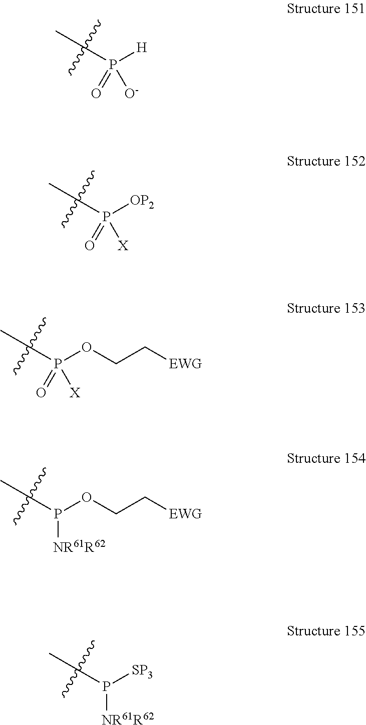

where the substituents of Structure 151, Structure 152, Structure 153, Structure 154 and Structure 155 above are: “P₂” and “P₃” are, independently, protecting groups (e.g., Bn, —CH₂CH₂SC(O)Ph), and where “EWG” is an electron withdrawing group (e.g., —CN, —NO₂), and where R⁶¹ and R⁶² are alkyl, substituted alkyl, aryl, substituted aryl, or together form a heterocycle with the nitrogen atom bound to the phosphorus atom (e.g., pyrrolidine, piperidine).

In one case, the deprotected, modified dimer in step “5” of the method recited above is one of the following structures:

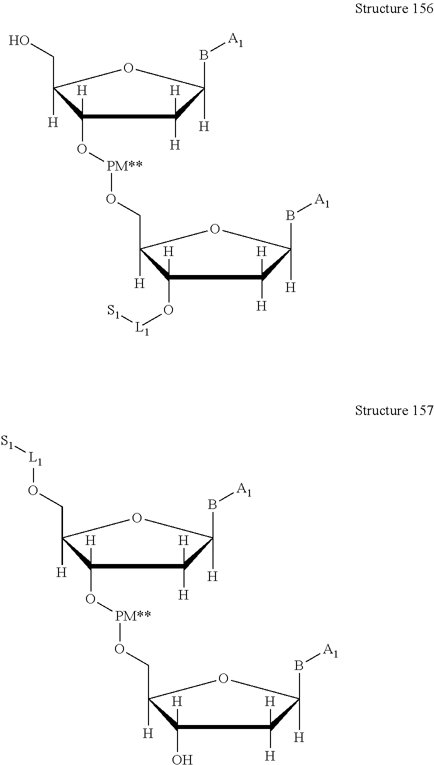

where “A₁” is —H or —C(O)OR⁴, and where R⁴ is tertiary alkyl (e.g., —C(O)OC(CH₃)₃), and where “B” is a nucleobase or nucleobase analogue, and where “PM**” is the phosphorus containing moiety, for example, selected from one of the following moieties: —P(O)(O—); —P(S)(O—); —P(O)(—CH₂CH₂-EWG)-; —P(S)(—CH₂CH₂-EWG)-, where L₁ is a linker or no chemical moiety, and S₁ is a solid support.

In one case, the oligomer in step “7” of the method recited above is of the following structure:

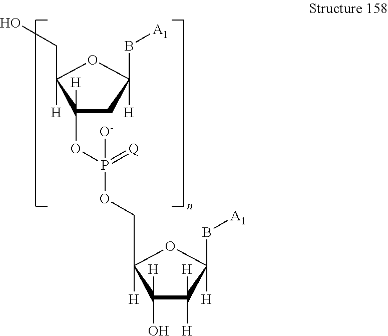

where the substituents of Structure 158 above are: “A₁” is —H or —C(O)OR⁶⁰, and where R⁶⁰ is tertiary alkyl (e.g., —C(O)OC(CH₃)₃), and where “B” is a nucleobase or nucleobase analogue, and where “Q” is O or S.

In another aspect, the present invention is directed to a method of synthesizing an oligonucleotide (or salts thereof). The method comprises the following steps:

1) coupling a compound to a solid support, either directly or through a linker, where the compound is of one of the following structures:

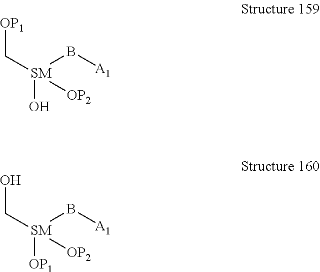

where the substituents of Structure 159 and Structure 160 above are: “P₁” and “P₂” are independently protecting groups, “B” is a nucleobase or nucleobase analogue, and “SM” is a sugar moiety or sugar moiety analogue, and “A₁” is H or —C(O)OR⁶⁰ where R⁶⁰ is tertiary alkyl (e.g., —C(O)OC(CH₃)₃) to provide a solid support bound compound of one of the following structures:

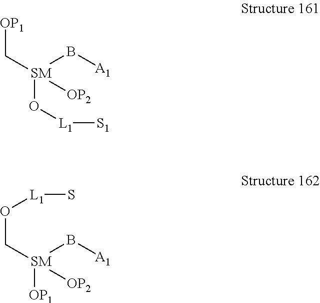

where the substituents of Structure 161 and Structure 162 above are: “P₁” and “P₂” are independently protecting groups, “B” is a nucleobase or nucleobase analogue, and “SM” is a sugar moiety or sugar moiety analogue, and “A₁” is H or —C(O)OR⁶⁰ where R⁶⁰ is tertiary alkyl (e.g., —C(O)OC(CH₃)₃), L₁ is a linker or no chemical moiety, and S₁ is a solid support;

2) deprotecting the solid support bound compound to provide a deprotected compound of one of the following structures:

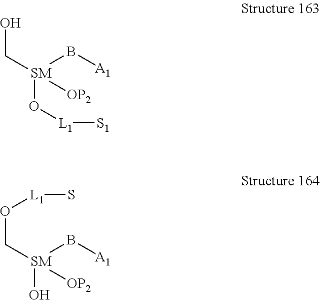

where the substituents of Structure 163 and Structure 164 above are: “P₂” is a protecting group, “B” is a nucleobase or nucleobase analogue, “SM” is a sugar moiety or sugar moiety analogue, and “A₁” is H or —C(O)OR⁶⁰ where R⁶⁰ is tertiary alkyl (e.g., —C(O)OC(CH₃)₃), L₁ is a linker or no chemical moiety, and S₁ is a solid support;

3) reacting the deprotected compound with a compound including a moiety comprising a phosphorus atom, wherein the compound is of one of the following structures:

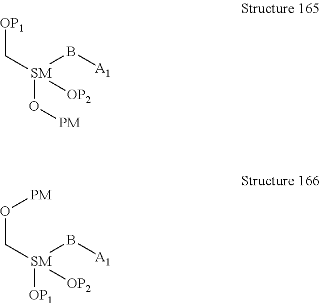

where the substituents of Structure 165 and Structure 166 above are: “P₁” and “P₂” are independently protecting groups, “B” is a nucleobase or nucleobase analogue, and “SM” is a sugar moiety or sugar moiety analogue, and “A₁” is H or —C(O)OR⁶⁰ where R⁶⁰ is tertiary alkyl (e.g., —C(O)OC(CH₃)₃), L₁ is a linker or no chemical moiety, and S₁ is a solid support, “PM” is the phosphorus containing moiety, to provide a dinucleotide of one of the following structures:

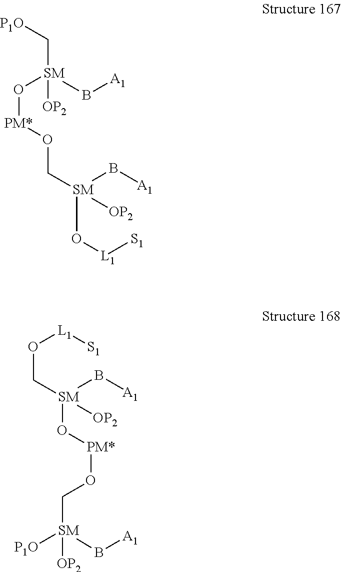

where the substituents of Structure 167 and Structure 168 above are: “P₁” and “P₂” are independently protecting groups, “B” is a nucleobase or nucleobase analogue, and “SM” is a sugar moiety or sugar moiety analogue, and “A₁” is H or —C(O)OR⁶⁰ where R⁶⁰ is tertiary alkyl (e.g., —C(O)OC(CH₃)₃), L₁ is a linker or no chemical moiety, and S₁ is a solid support, “PM*” is the phosphorus containing moiety after the reaction;

4) optionally, chemically modifying the phosphorus containing moiety to provide a modified dimer of one of the following structures:

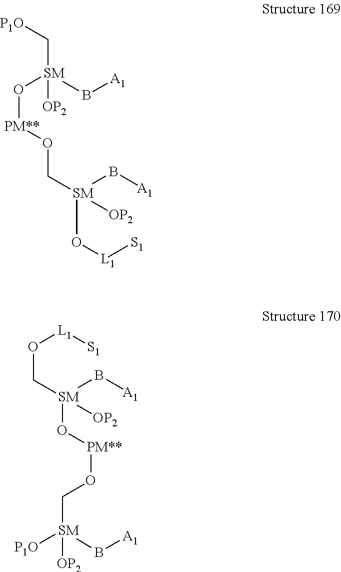

where the substituents of Structure 169 and Structure 170 above are: “P₁” and “P₂” are independently protecting groups, “B” is a nucleobase or nucleobase analogue, and “SM” is a sugar moiety or sugar moiety analogue, and “A₁” is H or —C(O)OR⁶⁰ where R⁶⁰ is tertiary alkyl (e.g., —C(O)OC(CH₃)₃), L₁ is a linker or no chemical moiety, and Sj is a solid support, “PM**” is a chemically modified phosphorus containing moiety;

5) optionally, deprotecting the dimer or modified dimer to provide a deprotected dimer or modified dimer of one of the following structures:

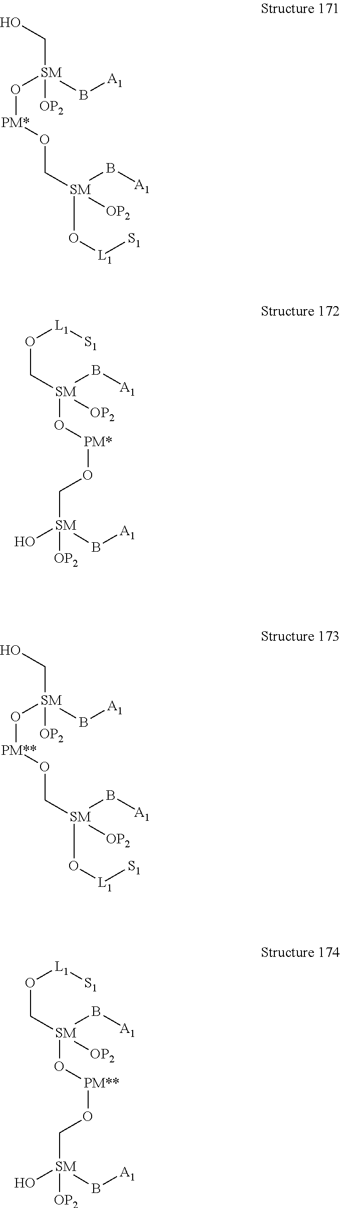

where the substituents of Structure 171, Structure 172, Structure 173 and Structure 174 above are: “P₂” is a protecting group, “B” is a nucleobase or nucleobase analogue, and “SM” is a sugar moiety or sugar moiety analogue, and “A₁” is H or —C(O)OR⁶⁰ where R⁶⁰ is tertiary alkyl (e.g., —C(O)OC(CH₃)₃), L₁ is a linker or no chemical moiety, and S₁ is a solid support, “PM*” is the phosphorus containing moiety after the coupling reaction, “PM**” is a chemically modified phosphorus containing moiety;

6) optionally repeating steps “3” and “4” to provide an oligomer or modified oligomer of one of the following structures:

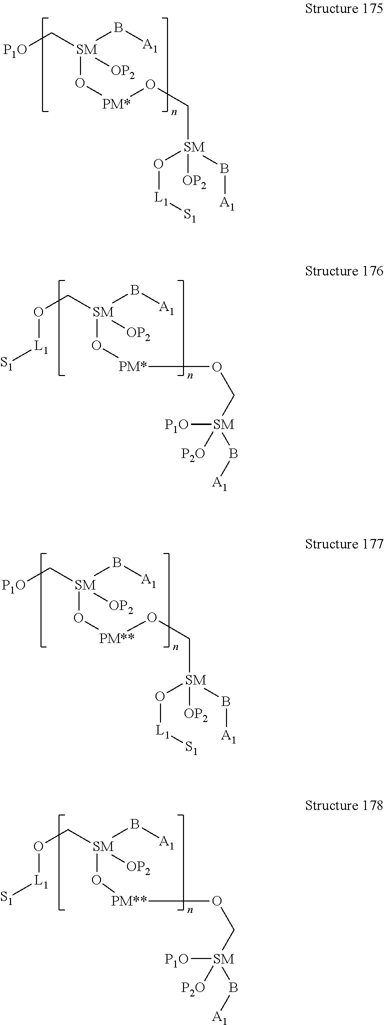

where the substituents for Structure 175, Structure 176, Structure 177 and Structure 178 above are: “P₁” and “P₂” are, independently, protecting groups, “B” is a nucleobase or nucleobase analogue, and “SM” is a sugar moiety or sugar moiety analogue, and “A₁” is H or —C(O)OR⁶ where R⁶⁰ is tertiary alkyl (e.g., —C(O)OC(CH₃)₃), L₁ is a linker or no chemical moiety, and S is a solid support, “PM*” is the phosphorus containing moiety after the coupling reaction, “PM**” is a chemically modified phosphorus containing moiety, “n” is an integer ranging from 1 to 200 (e.g., 1 to 25, 1 to 50, 1 to 75, etc.);

7) deprotecting the dimer, modified dimer, oligonucleotide or modified oligonucleotide, removing it from the solid support, and chemically modifying the “PM*” or “PM**” moiety to provide a compound of the following structure:

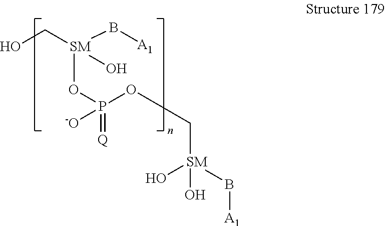

where “n” is an integer ranging from 1 to 200(e.g., 1 to 25, 1 to 50, 1 to 75, etc.), and where “B” is a nucleobase or nucleobase analogue, and where “SM” is a sugar moiety or sugar moiety analogue, and where at least one “A₁” is —C(O)OR⁶⁰ where R⁶⁰ is tertiary alkyl (e.g., —C(CH₃)₃), and where “Q” is O or S.

In one case, the compound coupled to the solid support in step “1” of the above recited method is one of the following structures:

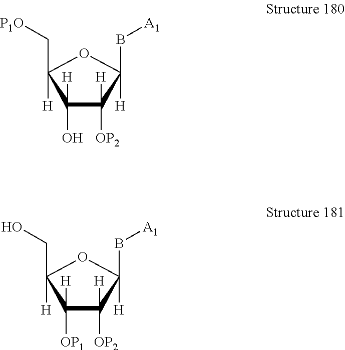

where the substituents of Structure 180 and Structure 181 above are: “P₁” and “P₂” are, independently, protecting groups, “B” is a nucleobase or nucleobase analogue, and “A₁” is —H or —C(O)OR⁶⁰, where R⁶⁰ is tertiary alkyl (e.g., —C(O)OC(CH₃)₃).

In another case, the compound coupled to the solid support in step “1” of the above recited method is one of the following structures:

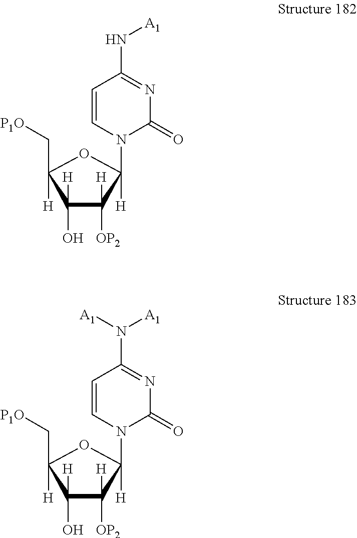

where the substituents of Structure 182 and Structure 183 above are: “P₁” and “P₂” are, independently, protecting groups, and “A₁” is —H or —C(O)OR⁶⁰, where R⁶⁰ is tertiary alkyl (e.g., —C(O)OC(CH₃)₃); or,

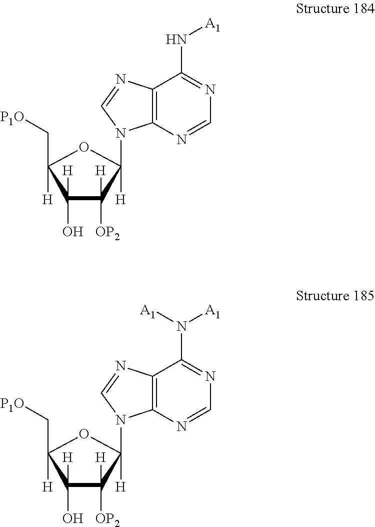

where the substituents of Structure 184 and Structure 185 above are: “P₁” and “P₂” are, independently, protecting groups, and “A₁” is —H or —C(O)OR⁶⁰, where R⁶⁰ is tertiary alkyl (e.g., —C(O)OC(CH₃)₃); or,

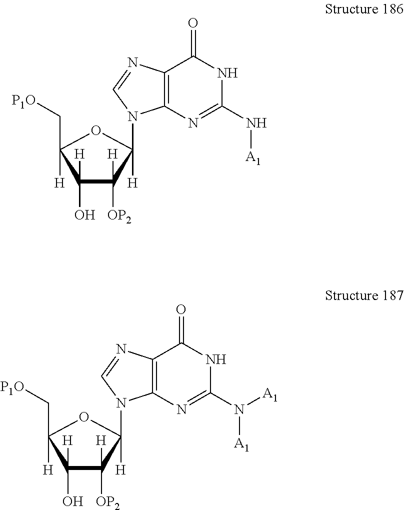

where the substituents of Structure 186 and Structure 187 above are: “P₁” and “P₂” are, independently, protecting groups, and “A₁” is —H or —C(O)OR⁶⁰, where R⁶⁰ is tertiary alkyl (e.g., —C(O)OC(CH₃)₃); or,

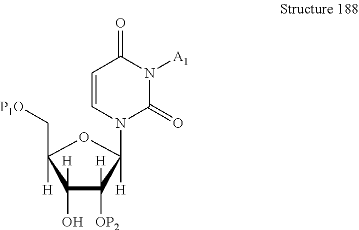

where the substituents of Structure 188 above are: “P₁” and “P₂” are, independently, protecting groups, and “A₁” is —H or —C(O)OR⁶⁰, where R⁶⁰ is tertiary alkyl (e.g., —C(O)OC(CH₃)₃).

In one case, the deprotected structure in step “2” of the method recited above is one of the following structures:

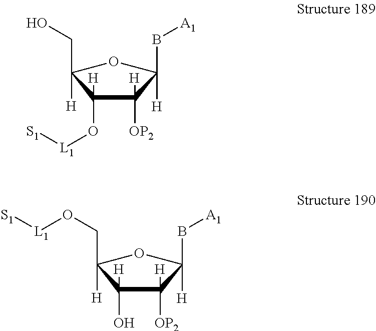

where the substituents of Structure 189 and Structure 190 above are: “P₂” is a protecting group, and where “B” is a nucleobase or nucleobase analogue, and where “A₁” is —H or —C(O)OR⁶⁰, where R⁶⁰ is tertiary alkyl (e.g., —C(O)OC(CH₃)₃), and where L₁ is a linker or no chemical entity, and S₁ is a solid support.

In one case, the deprotected structure in step “2” of the method recited above is one of the following structures:

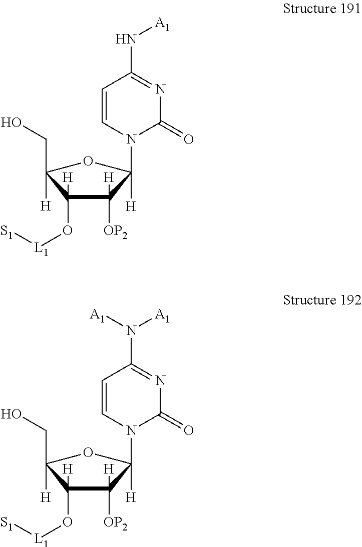

where the substituents of Structure 191 and Structure 192 above are: “P₂” is a protecting group, and where “A₁” is —H or —C(O)OR⁶⁰, where R⁶⁰ is tertiary alkyl (e.g., —C(O)OC(CH₃)₃), and where L₁ is a linker or no chemical moiety, and S₁ is a solid support; or

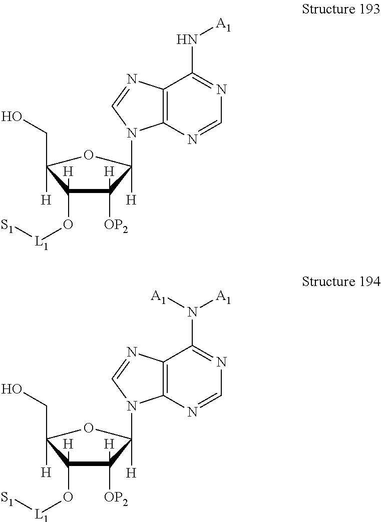

where the substituents of Structure 193 and Structure 194 above are: “P₂” is a protecting group, and where “A₁” is —H or —C(O)OR⁶⁰, where R⁶⁰ is tertiary alkyl (e.g., —C(O)OC(CH₃)₃), and where L₁ is a linker or no chemical moiety, and S₁ is a solid support; or

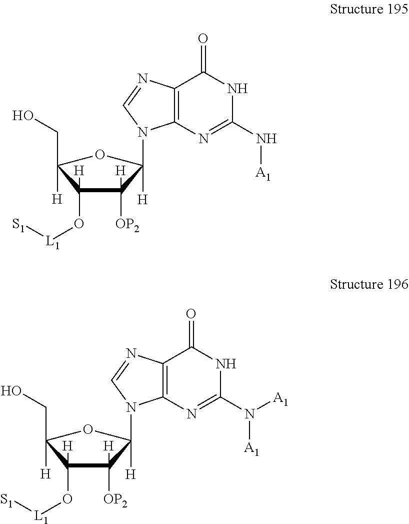

where the substituents of Structure 195 and Structure 196 above are: “P₂” is a protecting group, and where “A₁” is —H or —C(O)OR⁶⁰, where R⁶⁰ is tertiary alkyl (e.g., —C(O)OC(CH₃)₃), and where L₁ is a linker or no chemical moiety, and S₁ is a solid support; or

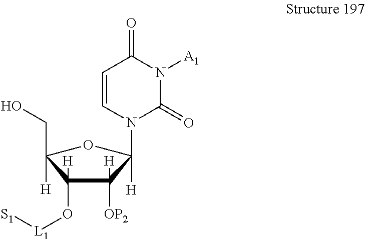

where the substituents of Structure 197 above are: “P₂” is a protecting group, and where “A₁” is —H or —C(O)OR⁶⁰, where R⁶⁰ is tertiary alkyl (e.g., —C(O)OC(CH₃)₃), and where L₁ is a linker or no chemical moiety, and S₁ is a solid support.

In one case, the compound including a moiety comprising a phosphorus atom in step “3” of the method recited above is of one of the following structures:

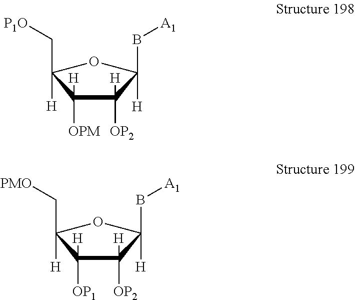

where the substituents of Structure 198 and Structure 199 above are: “P₁” and “P₂” are, independently, protecting groups, and where “A₁” is —H or —C(O)OR⁶⁰, where R⁶⁰ is tertiary alkyl (e.g., —C(O)OC(CH₃)₃), and where “B” is a nucleobase or nucleobase analogue, and where “PM” is a phosphorus containing moiety selected from one of the following moieties:

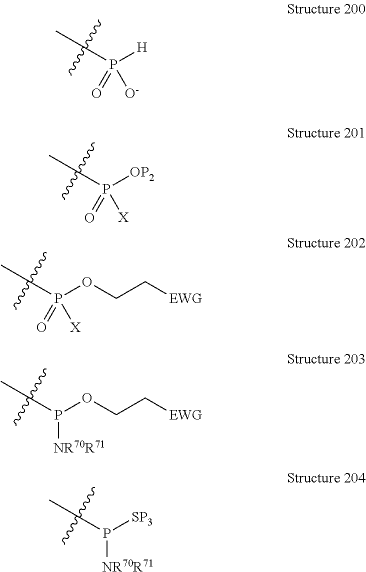

where the substituents of Structure 200, Structure 201, Structure 202, Structure 203 and Structure 204 above are: “P₃” and “P₄” are, independently, protecting groups (e.g., Bn, —CH₂CH₂SC(O)Ph), and where “EWG” is an electron withdrawing group (e.g., —CN, -PhNO₂), and where R⁷⁰ and R⁷¹ are alkyl, substituted alkyl, aryl, substituted aryl, or together form a heterocycle with the nitrogen atom bound to the phosphorus atom (e.g., pyrrolidine, piperidine).

In one case, the deprotected, modified dimer in step “5” of the method recited above is one of the following structures:

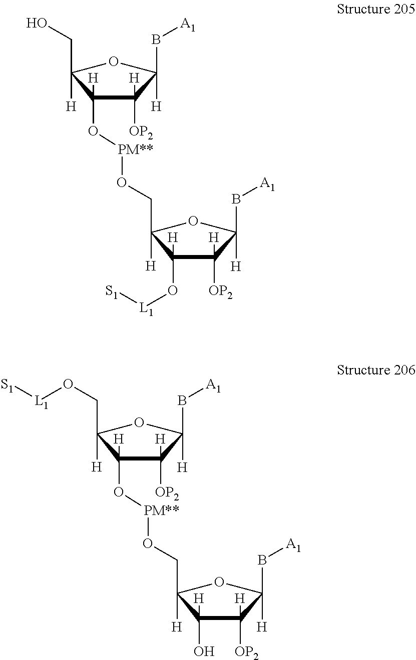

where the substituents of Structure 205 and Structure 206 above are: “P₂” is a protecting group, and where “A₁” is —H or —C(O)OR⁶⁰, and where R⁶⁰ is tertiary alkyl (e.g., —C(O)OC(CH₃)₃), and where “B” is a nucleobase or nucleobase analogue, and where “PM**” is the phosphorus containing moiety, for example, selected from one of the following moieties: —P(O)(O—); —P(S)(O—); —P(O)(—CH₂CH₂-EWG)-; —P(S)(—CH₂CH₂-EWG)-, where “EWG” is an electron withdrawing group (e.g., —CN, —NO₂).

In one case, the oligomer in step “7” of the method recited above is of the following structure:

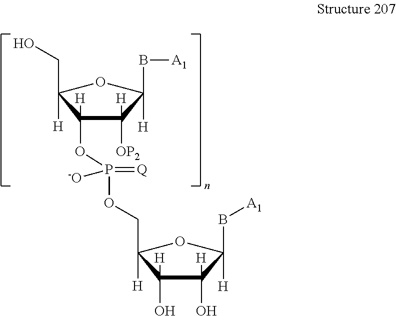

where the substituents of Structure 207 above are: “A₁” is —H or —C(O)OR⁶⁰, and where R⁶⁰ is tertiary alkyl (e.g., —C(O)OC(CH₃)₃), and where “B” is a nucleobase or nucleobase analogue, and where “Q” is O or S.

In another aspect, the present invention is directed to a method of amplifying DNA using the polymerase chain reaction (PCR). The method involves using one or more deoxynucleotide triphosphates having at least one thermally labile protecting group on a nitrogen atom on or within the ring structure of a nucleobase, where the protecting group is of the structure —C(O)OR⁶⁰ where R⁶⁰ is a tertiary alkyl (e.g., —C(CH₃)₃).

For a discussion of PCR, see: U.S. Pat. No. 8,133,669; U.S. Pat. No. 4,683,195; U.S. Pat. No. 4,683,202; U.S. Pat. No. 4,800,159; U.S. Pat. No. 4,965,188; U.S. Pat. No. 5,008,182; U.S. Pat. No. 5,176,995; U.S. Pat. No. 6,040,166; U.S. Pat. No. 6,197,563. The preceding references are hereby incorporated-by-reference into this document for all purposes.

In another aspect, the present invention is directed to a method of amplifying DNA using PCR, where the method comprises the following steps:

1) providing a reaction mixture comprising target DNA (i.e., the DNA to be amplified), DNA polymerase, primers and deoxynucleotide triphosphates (dNTPs), where one or more of the dNTPs is of one of the following structures:

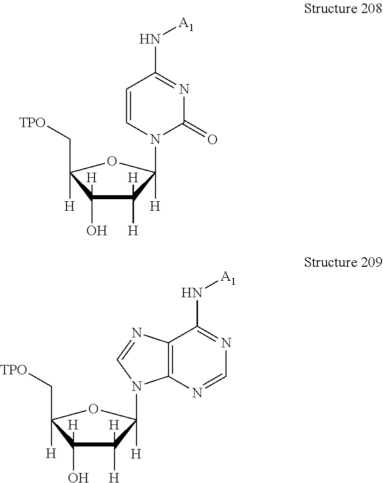

where the substituents of Structure 208 and Structure 209 above are: “TP” is triphosphate, “A₁” is —C(O)OR⁶⁰, where R⁶⁰ is tertiary alkyl (e.g., —C(O)OC(CH₃)₃);

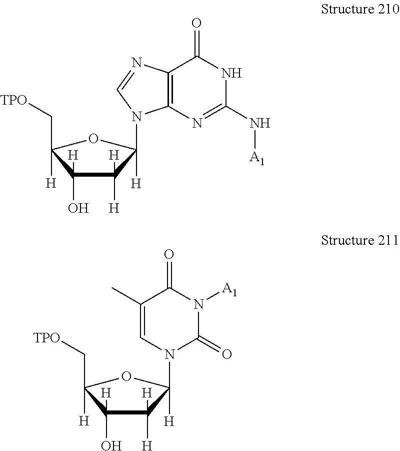

where the substituents of Structure 210 and Structure 211 above are: “TP” is triphosphate, “A₁” is —C(O)OR⁶⁰, where R⁶⁰ is tertiary alkyl (e.g., —C(O)OC(CH₃)₃);

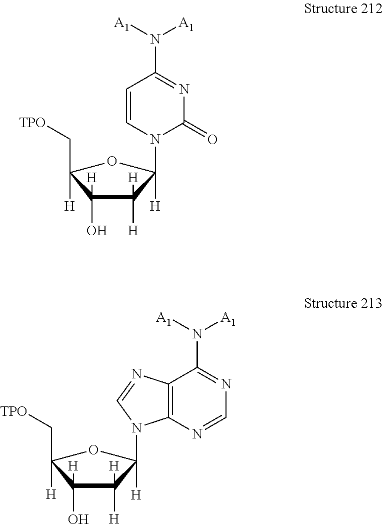

where the substituents of Structure 212 and Structure 213 above are: “TP” is triphosphate, “A₁” is —C(O)OR⁶⁰, where R⁶⁰ is tertiary alkyl (e.g., —C(O)OC(CH₃)₃);

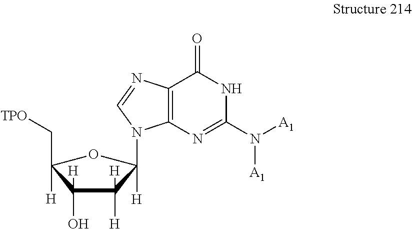

where the substituents of Structure 214 above are: “TP” is triphosphate, “A₁” is —C(O)OR⁶⁰, where R⁶⁰ is tertiary alkyl (e.g., —C(O)OC(CH₃)₃);

2) heating the reaction mixture (e.g., 94° C. to 98° C.) for a period of time (e.g., one minute) to denature the target DNA, thereby providing a single-stranded DNA template;

3) lowering the reaction temperature (e.g., 50° C. to 65° C.) of the reaction mixture for a period of time (e.g., 20 to 40 seconds), which allows annealing of primers to the single-stranded DNA template to provide a primer-template complex and binding of the DNA polymerase to the primer-template complex;

4) heating the reaction mixture (e.g., 75° C. to 80° C.), allowing the DNA polymerase to synthesize a DNA strand complementary to the target DNA by adding the dNTPs to the DNA template in the 5′ to 3′ direction;

5) optionally holding the temperature of the reaction mixture at 70° C. to 74° C. to ensure extension of any remaining single-stranded DNA.

In another aspect, the present invention is directed to a method of amplifying DNA using the polymerase chain reaction (PCR). The method involves using one or more primers (i.e., oligonucleotides targeted to a specific DNA sequence) having one or more thermally labile protecting groups on a nitrogen atom on or within the ring structure of a nucleobase of the primer, where the protecting group is of the structure —C(O)OR⁴ where R⁴ is a tertiary alkyl (e.g., —C(CH₃)₃).

In another aspect, the present invention is directed to a method of amplifying DNA using PCR, where the method comprises the following steps:

1) providing a reaction mixture comprising target DNA (i.e., the DNA to be amplified), DNA polymerase, primers and deoxynucleotide triphosphates (dNTPs), where one or more of the primers is of the following structure:

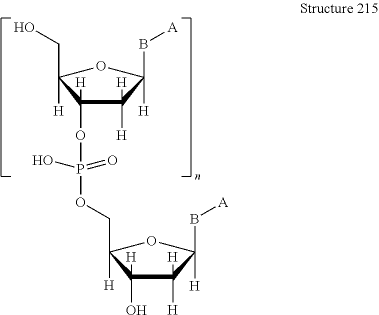

where the substituents of Structure 215 above are: “n” is an integer between 1 and 50, and where “B” is a nucleobase, and where “A” is either H or a thermally labile protecting group of the structure —C(O)OR⁶⁰ where R⁶⁰ is tertiary alkyl (e.g., —C(CH₃)₃), provided that at least one “A” is a thermally labile protecting group;

2) heating the reaction mixture (e.g., 94° C. to 98° C.) for a period of time (e.g., one minute) to denature the target DNA, thereby providing a single-stranded DNA template;

3) lowering the reaction temperature (e.g., 50° C. to 65° C.) of the reaction mixture for a period of time (e.g., 20 to 40 seconds), which allows annealing of primers to the single-stranded DNA template to provide a primer-template complex and binding of the DNA polymerase to the primer-template complex;

4) heating the reaction mixture (e.g., 75° C. to 80° C.), allowing the DNA polymerase to synthesize a DNA strand complementary to the target DNA by adding the dNTPs to the DNA template in the 5′ to 3′ direction;

5) optionally holding the temperature of the reaction mixture at 70° C. to 74° C. to ensure extension of any remaining single-stranded DNA.

In another aspect, the present invention is directed to a method of making nucleoside, or nucleoside analogue, triphosphates, where the nucleoside or nucleoside analogue triphosphate includes at least one thermally labile protecting group. The method comprises the steps of:

1) adding a monophosphorus reagent, and optionally a condensing agent (e.g., carbonyldiimidazole), to a reaction mixture comprising a nucleoside or nucleoside analogue, where the analogue is of the following structure:

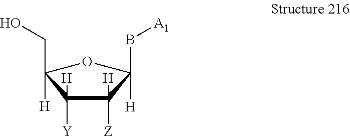

where the substituents of Structure 216 above are: Y is OP¹ where P¹ is a protecting group or —H, Z is H or OP² where P² is a protecting group or —H, B is a nucleobase or a nucleobase analogue, and A is a thermally labile protecting group of the structure —C(O)OR⁶⁰ where R⁶⁰ is a tertiary alkyl (e.g., —C(CH₃)₃), to provide a mono-phosphorylated intermediate of the following structure:

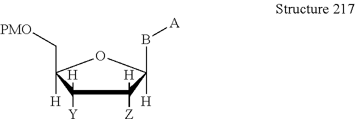

where the substituents of Structure 217 above are: Y is OP¹ where P¹ is a protecting group, Z is H or OP² where P² is a protecting group, B is a nucleobase or a nucleobase analogue, and A is a thermally labile protecting group of the structure —C(O)OR⁶⁰ where R⁶⁰ is a tertiary alkyl (e.g., —C(CH₃)₃), “PM” is a moiety comprising a single phosphorus atom;

2) adding a polyphosphorus reagent to the phosphorylated intermediate to provide a poly-phosphorylated intermediate of the following structure:

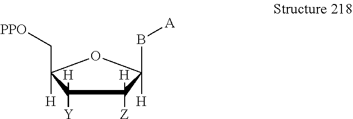

where the substituents of Structure 218 above are: Y is OP¹ where P¹ is a protecting group, Z is H or OP² where P² is a protecting group, B is a nucleobase or a nucleobase analogue, and A is a thermally labile protecting group of the structure —C(O)OR⁶⁰ where R⁶⁰ is a tertiary alkyl (e.g., —C(CH₃)₃), “PP” is a moiety comprising multiple phosphorus atoms;

3) hydrolyzing the poly-phosphorylated intermediate and removing P₁ to provide a nucleoside triphosphate or nucleoside analogue triphosphate of the following structure:

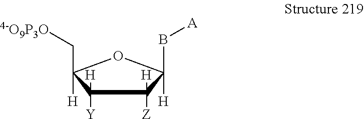

where the substituents of Structure 219 above are: Y is OP¹ where P¹ is a protecting group, Z is H or OP² where P² is a protecting group, B is a nucleobase or a nucleobase analogue, and A is a thermally labile protecting group of the structure —C(O)OR⁶⁰ where R⁶⁰ is a tertiary alkyl (e.g., —C(CH₃)₃).

In one case, the monophosphorus reagent used in step “1” of the method recited above is selected from the following: POCl₃; and,

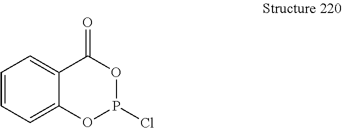 [SMILES: O=C1OP(Cl)Oc2ccccc21]

In one case, the nucleoside or nucleoside analogue of step “1” of the method recited above is of one of the following structures:

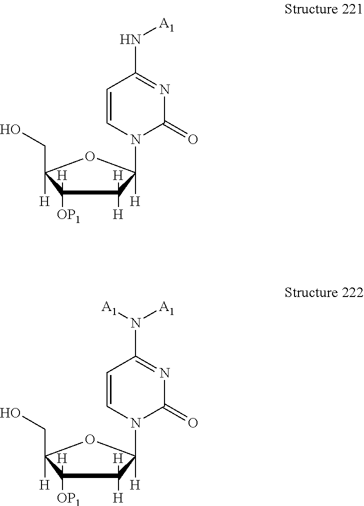

where the substituents of Structure 221 and Structure 222 above are: P₁ is a protecting group, and A is a thermally labile protecting group of the structure —C(O)OR⁶⁰ where R⁶⁰ is a tertiary alkyl (e.g., —C(CH₃)₃);

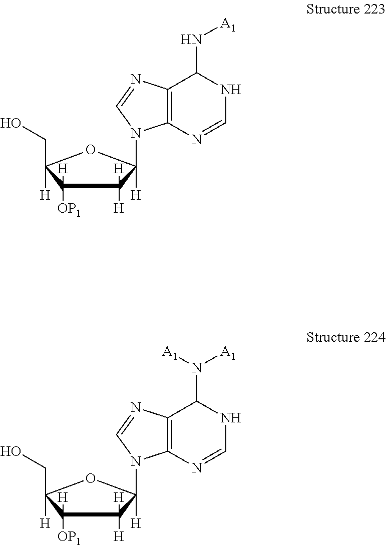

where the substituents of Structure 223 and Structure 224 above are: P₁ is a protecting group, and A is a thermally labile protecting group of the structure —C(O)OR⁶⁰ where R⁶⁰ is a tertiary alkyl (e.g., —C(CH₃)₃);

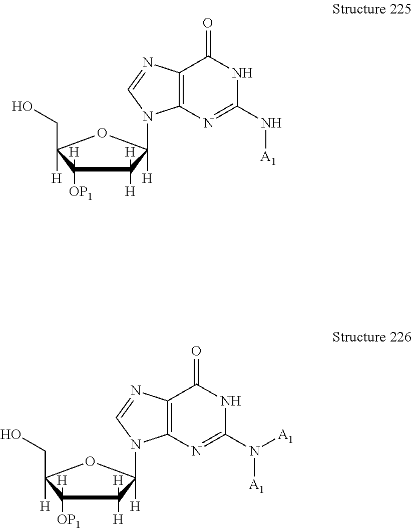

where the substituents of Structure 225 and Structure 226 above are: P₁ is a protecting group, and A is a thermally labile protecting group of the structure —C(O)OR⁶⁰ where R⁶⁰ is a tertiary alkyl (e.g., —C(CH₃)₃);

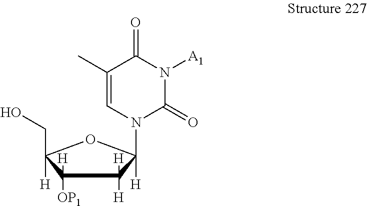

where the substituents of Structure 227 above are: P₁ is a protecting group, and A is a thermally labile protecting group of the structure —C(O)OR⁶⁰ where R⁶⁰ is a tertiary alkyl (e.g., —C(CH₃)₃).

In one case, the polyphosphorus reagent of step “2” of the method recited above is one of the following structures: (n-Bu₃NH)₂H₂P₂O₇; and, P₂O₇⁴⁻.

In one case, the poly-phosphorylated intermediate of step “2” of the method recited above is of the following structure:

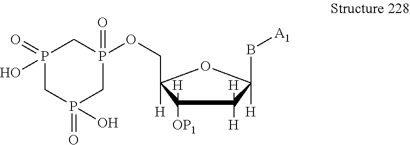

where the substituents of Structure 228 above are: P₁ is a protecting group, B is a nucleobase or a nucleobase analogue, and A₁ is a thermally labile protecting group of the structure —C(O)OR⁶⁰ where R⁶⁰ is a tertiary alkyl (e.g., —C(CH₃)₃).

In one case, the nucleoside triphosphate of the method recited above is one of the following:

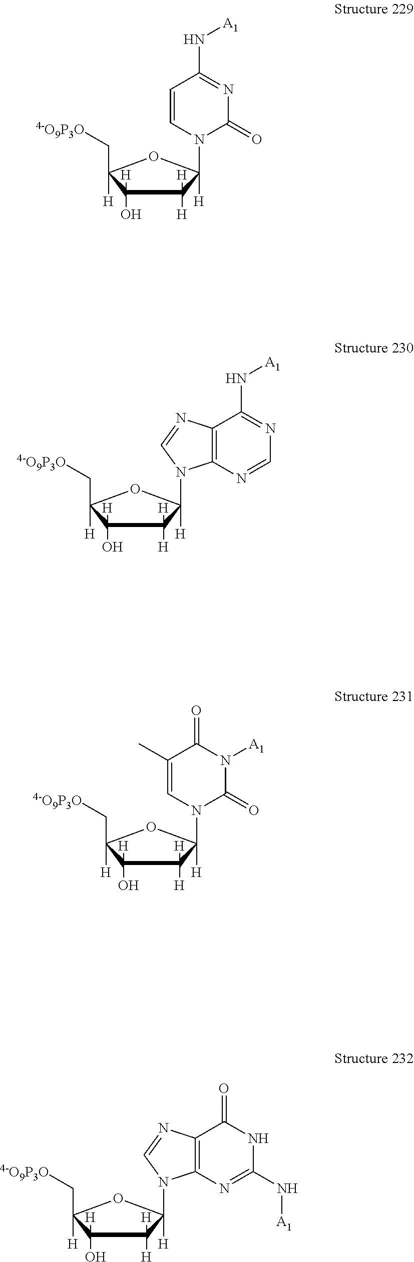

where the substituent of Structure 229, Structure 230, Structure 231 and Structure 232 above is: A₁ is a thermally labile protecting group of the structure —C(O)OR⁶⁰ where R⁶⁰ is a tertiary alkyl (e.g., —C(CH₃)₃).

For a discussion of triphosphate synthesis, see: Gregor S. Cremosnik, Alexandre Hofer and Henning J. Jessen Angew. Chem. Int. Ed., 2014, 53, 286; Malwina Strenkowska, Przemyslaw Wanat, Marcin Ziemniak, Jacek Jemielity and Joanna Kowalska Org. Lett., 2012, 14, 4782; Tobias Santner, Vanessa Siegmund, Andreas Marx and Ronald Micura Bioorganic & Medicinal Chemistry, 2012, 20, 2416; Julianne Caton-Williams, Bilal Fiaz, Rudiona Hoxhaj, Matthew Smith and Zhen Huang Sci. China Chem., 2012, 55, 80; Gregor S. Cremosnik, Alexandre Hofer and Henning J. Jessen Angew. Chem., 2014, 126, 290; Qi Sun, Shanshan Gong, Jian Sun, Si Liu, Qiang Xiao and Shouzhi Pu J. Org. Chem., 2013, 78, 8417; Julianne Caton-Williams, Matthew Smith, Nicolas Carrasco and Zhen Huang Org. Lett., 2011, 13, 4156; Julianne caton-Williams, Lina Lin, Matthew Smith and Zhen Huang Chem Commun., 2011, 47, 8142-8144. The preceding references are hereby incorporated-by-reference into this document for all purposes.

In another aspect, the present invention is directed to a method of treating a disease where the method comprises the following steps:

1) administering a therapeutic amount of a compound to a patient in need thereof, wherein the compound comprises a nucleotide, nucleotide analogue, nucleoside or nucleoside analogue and one or more thermally labile protecting groups, where at least one of the thermally labile protecting groups is of the structure —C(O)OR⁸, and where R⁸ is a tertiary alkyl group (e.g., —C(CH₃)₃);

2) applying energy to one or more areas of the patient, resulting in an increase of temperature in the one or more areas and the subsequent thermal deprotection of the nucleotide, nucleotide analogue, nucleoside or nucleoside analogue;

thereby treating the disease.

For a discussion of certain thermolabile protecting groups, see: Chmielewski, M. et al. New J. Chem., 2012, 36, 603-12. U.S. Pat. No. 8,133,669; U.S. Pat. No. 7,355,037; U.S. Pat. No. 6,762,298. The preceding references are hereby incorporated-by-reference into this document for all purposes.

In one case, the therapeutic compound is of one of the following structures:

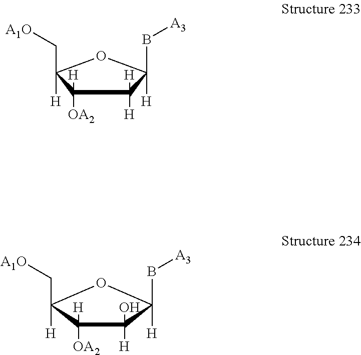

where the substituents of Structure 233 and Structure 234 above are: “A₁”, “A₂” and “A₃” are, independently —H or a thermolabile protecting group, provided that at least one of A₁, A₂ or A₃ is a thermolabile protecting group of the structure —C(O)OR⁶⁰, where R⁶⁰ is a tertiary alkyl (e.g., —C(O)OC(CH₃)₃); and where “B” is a nucleobase or nucleobase analogue.

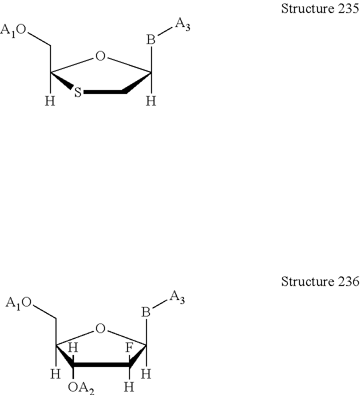

where the substituents of Structure 235 and Structure 236 above are: “A₁”, “A₂” and “A₃” are, independently —H or a thermolabile protecting group, provided that at least one of A₁, A₂ or A₃ is a thermolabile protecting group of the structure —C(O)OR⁶⁰, where R⁶⁰ is a tertiary alkyl (e.g., —C(O)OC(CH₃)₃); and where “B” is a nucleobase or nucleobase analogue;

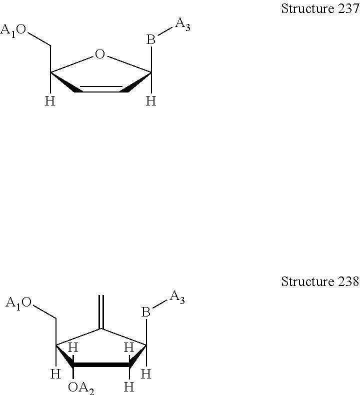

where the substituents of Structure 237 and Structure 238 above are: “A₁”, “A₂” and “A₃” are, independently —H or a thermolabile protecting group, provided that at least one of A₁, A₂ or A₃ is a thermolabile protecting group of the structure —C(O)OR⁶⁰, where R⁶⁰ is a tertiary alkyl (e.g., —C(O)OC(CH₃)₃); and where “B” is a nucleobase or nucleobase analogue;

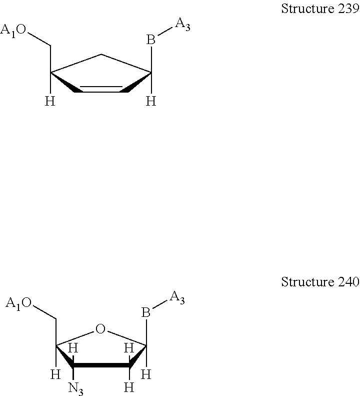

where the substituents of Structure 239 and Structure 240 above are: “A₁”, “A₂” and “A₃” are, independently —H or a thermolabile protecting group, provided that at least one of A₁, A₂ or A₃ is a thermolabile protecting group of the structure —C(O)OR⁶⁰, where R⁶⁰ is a tertiary alkyl (e.g., —C(O)OC(CH₃)₃); and where “B” is a nucleobase or nucleobase analogue;

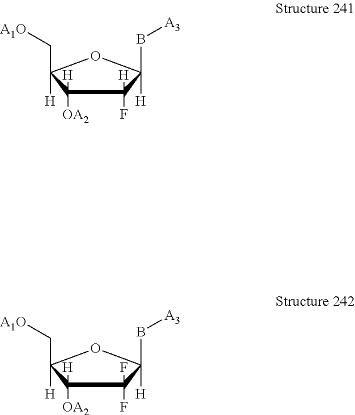

where the substituents of Structure 241 and Structure 242 above are: “A₁”, “A₂” and “A₃” are, independently —H or a thermolabile protecting group, provided that at least one of A₁, A₂ or A₃ is a thermolabile protecting group of the structure —C(O)OR⁶⁰, where R⁶⁰ is a tertiary alkyl (e.g., —C(O)OC(CH₃)₃); and where “B” is a nucleobase or nucleobase analogue;

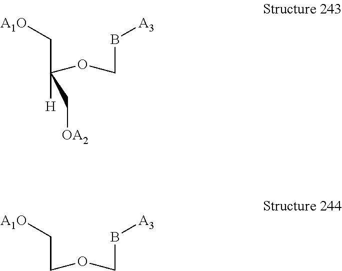

where the substituents of Structure 243 and Structure 244 above are: “A₁”, “A₂” and “A₃” are, independently —H or a thermolabile protecting group, provided that at least one of A₁, A₂ or A₃ is a thermolabile protecting group of the structure —C(O)OR⁴, where R⁴ is a tertiary alkyl (e.g., —C(O)OC(CH₃)₃); and where “B” is a nucleobase or nucleobase analogue;

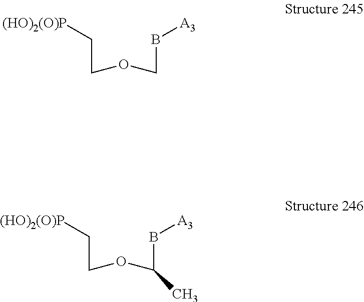

where the substituents of Structure 245 and Structure 246 above are: “A₃” is a thermolabile protecting group of the structure —C(O)OR⁶⁰, where R⁶⁰ is a tertiary alkyl (e.g., —C(O)OC(CH₃)₃); and where “B” is a nucleobase or nucleobase analogue;

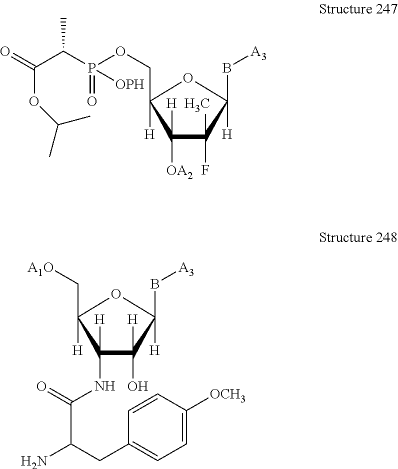

where the substituents of Structure 247 and Structure 248 above are: “A₁”, “A₂” and “A₃” are, independently —H or a thermolabile protecting group, provided that at least one of A₁, A₂ or A₃ is a thermolabile protecting group of the structure —C(O)OR⁶⁰, where R⁶⁰ is a tertiary alkyl (e.g., —C(O)OC(CH₃)₃); and where “B” is a nucleobase or nucleobase analogue;

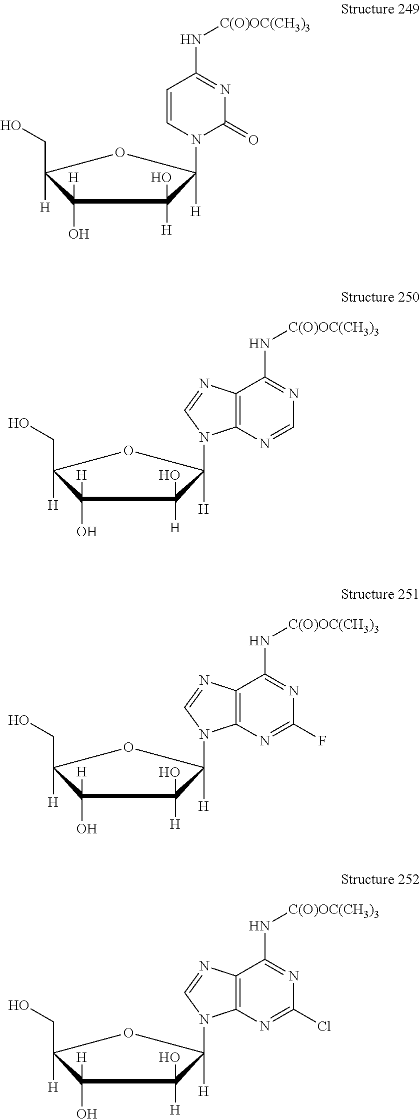

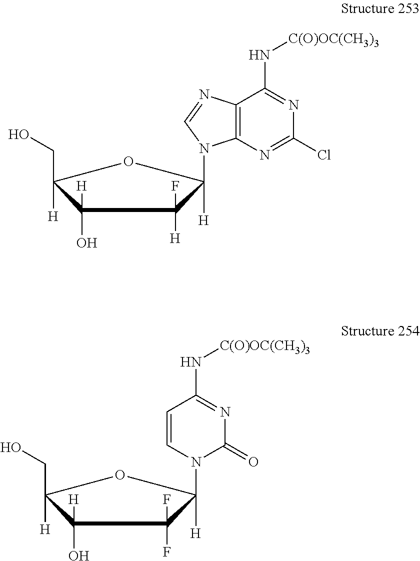

In one case, the thermal energy is applied to one or more areas of the patient using one or more of the following methods: microwave phased array or single applicator hyperthermia as discussed in U.S. Pat. No. 6,725,095, U.S. Pat. No. 6,807,446 and U.S. Pat. No. 6,768,925, which are incorporate-by-reference for all purposes into this document.

In another aspect, the present invention is directed to a method of treating a disease where the method comprises the following steps:

1) administering a therapeutic amount of a compound to a patient in need thereof, wherein the compound comprises an oligonucleotide (or salt thereof) and one or more thermally labile protecting groups, where at least one of the thermally labile protecting groups is of the structure —C(O)OR⁶⁰, and where R⁶⁰ is a tertiary alkyl group (e.g., —C(CH₃)₃);

2) applying energy to one or more areas of the patient, resulting in an increase of temperature in the one or more areas and the subsequent thermal deprotection of the oligonucleotide;

thereby treating the disease.

In one case, the therapeutic compound is either Fomivirsen or Mipomersen to which is attached one or more thermally labile protecting groups of the structure —C(O)OR⁸, where R⁸ is a tertiary alkyl group (e.g., —C(CH₃)₃).

In one case, the thermal energy is applied to one or more areas of the patient using one or more of the following methods: microwave phased array or single applicator hyperthermia as discussed in U.S. Pat. No. 6,725,095, U.S. Pat. No. 6,807,446 and U.S. Pat. No. 6,768,925, which are incorporate-by-reference for all purposes into this document.

The present invention is further directed to a method of deprotecting nucleosides, nucleoside analogues, nucleotides and nucleotide analogues. The protected compounds are of the structure: XO-SM-B-A. Substituent “X” is H, a protecting group, a solid support, a phosphorus containing moiety or salts thereof. “SM” is a sugar moiety or an analogue of a sugar moiety. “B” is a base moiety of an analogue of a base moiety. “A” is one or more moieties attached to one or more nitrogen atoms on or within the base moiety and is of the structure —C(O)OR⁶⁰, wherein R⁶⁰ is a tertiary alkyl group.

The deprotection method comprises heating the compound in the presence of a solvent (e.g., water). In certain cases, the pH of the solvent is between 6.0 and 9.0—e.g., between 6.5 to 7.5, 6.75 to 7.25, 6.90 to 7.10, or approximately 7.0. In other cases, the pH of the solvent is above 7.0—e.g., 7.0 to 10.0, 7.0 to 9.0 or 7.0 to 8.0. The temperature to which the compound is heated ranges from 90° C. to 100° C. Oftentimes it ranges from 91° C. to 99° C., 92° C. to 97° C., 93° C. to 95° C. In certain cases, the temperature is 94° C. The temperature is maintained for a period less than one hour. Oftentimes it is maintained for less than 45 minutes or 30 minutes. In certain cases it is maintained for less than 20 minutes.

The deprotection method results in removal of more than 90 percent of the —C(O)OR¹ protecting groups. Oftentimes it results in removal of more than 92.5 percent or 95 percent of the protecting groups. In certain cases, it results in removal of more than 97.5 percent or 99 percent of the protecting groups.

The deprotection method further results in less than 5 percent degradation of the compound. Oftentimes it results in less than 4 percent or 3 percent degradation of the compound. In certain cases it results in less than 2 percent or 1 percent of the compound.

In another method, the compound XO-SM-B-A is deprotected in the presence of solvent by use of microwave technology. See, for example, Culf et al., Oligonucleotides 18:81-92 (2008), and Kumar et al., Nucleic Acids Research, 1997, Vol. 25, No. 24, pp. 5127-5129, both of which are incorporated by reference into this document. The pH of the solvent is typically greater than 6.0 or equal to or greater than 7.0—e.g., 7.0 to 7.5, 7.5 to 8.0, 8.0 to 8.5, 8.5 to 9.0. The temperature of the solvent in the microwave temperature is oftentimes less than 55° C.—e.g., less than 50° C., less than 45° C., less than 40° C., less than 35° C., or less than 30° C. In certain cases, either ammonia or an amine are included in the reaction mixture of the deprotection. Nonlimiting examples of amines include monoalkyl amines such as methyl amine, ethyl amine, propyl amine, ethanolamine, and dialkyl amines such as dimethyl amine, diethyl amine, and other amines such as DBU. In certain cases, the deprotection step takes less than 30 minutes to be more than 90 percent complete. Oftentimes, the deprotection step takes less than 25 minutes, 20 minutes, 15 minutes, 10 minutes or 5 minutes to be more than 90 percent complete.

In another method, the compound XO-SM-B-A is deprotected in the absence of solvent. The compound is heated to a temperature ranging from 90° C. to 100° C. Oftentimes it ranges from 91° C. to 97° C., 92° C. to 96° C., 93° C. to 95° C. In certain cases, the temperature is 94° C. The temperature is maintained for a period less than one hour. Oftentimes it is maintained for less than 45 minutes or 30 minutes. In certain cases it is maintained for less than 20 minutes.

The solventless deprotection method results in removal of more than 90 percent of the —C(O)OR¹ protecting groups. Oftentimes it results in removal of more than 92.5 percent or 95 percent of the protecting groups. In certain cases, it results in removal of more than 97.5 percent or 99 percent of the protecting groups.

The solventless deprotection method further results in less than 5 percent degradation of the compound. Oftentimes it results in less than 4 percent or 3 percent degradation of the compound. In certain cases it results in less than 2 percent or 1 percent of the compound.

The present invention is further directed to a method of deprotecting oligonucleotides or oligonucleotide analogues. The protected compounds are of the structure:

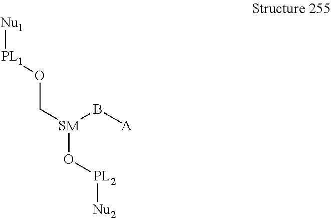

where the substituents of Structure 255 above are: “PL₁” and “PL₂” are, independently, either H or —P(O)(OH)O— or an analogue thereof, and where “Nu₁” and “Nu₂” are, independently, no substituent, a nucleoside or nucleoside analogue, or an oligonucleotide (or salts thereof), and where “SM” is a sugar moiety or sugar moiety analogue, and where “B” is a nucleobase or nucleobase analogue, “A” is one or more moieties attached to one or more nitrogen atoms on or within the nucleobase moiety and is of the structure —C(O)OR⁶⁰, wherein R⁶⁰ is a tertiary alkyl group.

The oligonucleotide, or oligonucleotide analogue, deprotection method comprises heating the compound in the presence of a solvent (e.g., water). In certain cases, the pH of the solvent is between 6.0 and 9.0—e.g., between 6.5 to 7.5, 6.75 to 7.25, 6.90 to 7.10, or approximately 7.0.

In other cases, the pH of the solvent is above 7.0—e.g., 7.0 to 10.0, 7.0 to 9.0 or 7.0 to 8.0. The temperature to which the compound is heated ranges from 90° C. to 100° C. Oftentimes it ranges from 91° C. to 99° C., 92° C. to 97° C., 93° C. to 95° C. In certain cases, the temperature is 94° C.

In certain cases, the temperature is 94° C. The temperature is maintained for a period less than one hour. Oftentimes it is maintained for less than 45 minutes or 30 minutes. In certain cases it is maintained for less than 20 minutes.

The deprotection method results in removal of more than 90 percent of the oligonucleotide/analogue —C(O)OR¹ protecting groups. Oftentimes it results in removal of more than 92.5 percent or 95 percent of the protecting groups. In certain cases, it results in removal of more than 97.5 percent or 99 percent of the protecting groups.

The deprotection method further results in less than 5 percent degradation of the oligonucleotide or oligonucleotide analogue. Oftentimes it results in less than 4 percent or 3 percent degradation of the compound. In certain cases it results in less than 2 percent or 1 percent of the compound.

In another method, an oligonucleotide comprising a protecting group of structure —C(O)OR⁶⁰, where R⁶⁰ is tertiary alkyl (e.g., C(CH₃)₃), is deprotected in the presence of solvent by use of microwave technology. See, for example, Culf et al., Oligonucleotides 18:81-92 (2008), and Kumar et al., Nucleic Acids Research, 1997, Vol. 25, No. 24, pp. 5127-5129, both of which are incorporated-by-reference into this document. The pH of the solvent is typically greater than 6.0 or equal to or greater than 7.0—e.g., 7.0 to 7.5, 7.5 to 8.0, 8.0 to 8.5, 8.5 to 9.0. The temperature of the solvent in the microwave temperature is oftentimes less than 55° C.—e.g., less than 50° C., less than 45° C., less than 40° C., less than 35° C., or less than 30° C. In certain cases, either ammonia or an amine are included in the reaction mixture of the deprotection. Nonlimiting examples of amines include monoalkyl amines such as methyl amine, ethyl amine, propyl amine, ethanolamine, and dialkyl amines such as dimethyl amine, diethyl amine, and other amines such as DBU. In certain cases, the deprotection step takes less than 30 minutes to be more than 90 percent complete. Oftentimes, the deprotection step takes less than 25 minutes, 20 minutes, 15 minutes, 10 minutes or 5 minutes to be more than 90 percent complete.

In another method, the compound

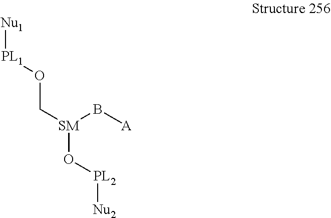

where the substituents of Structure 256 above are: “PL₁” and “PL₂” are, independently, either H or —P(O)(OH)O— or an analogue thereof, and where “Nu₁” and “Nu₂” are, independently, no substituent, a nucleoside or nucleoside analogue, or an oligonucleotide, and where “SM” is a sugar moiety or sugar moiety analogue, and where “B” is a nucleobase or nucleobase analogue, “A” is one or more moieties attached to one or more nitrogen atoms on or within the nucleobase moiety and is of the structure —C(O)OR⁶, wherein R⁶ is a tertiary alkyl group, is deprotected in the absence of solvent. The compound is heated to a temperature ranging from 90° C. to 100° C. Oftentimes it ranges from 91° C. to 97° C., 92° C. to 96° C., 93° C. to 95° C. In certain cases, the temperature is 94° C. The temperature is maintained for a period less than one hour. Oftentimes it is maintained for less than 45 minutes or 30 minutes. In certain cases it is maintained for less than 20 minutes.

The solventless deprotection method of the oligonucleotide or analogue results in removal of more than 90 percent of the —C(O)OR¹ protecting groups. Oftentimes it results in removal of more than 92.5 percent or 95 percent of the protecting groups. In certain cases, it results in removal of more than 97.5 percent or 99 percent of the protecting groups.

The solventless deprotection method further results in less than 5 percent degradation of the oligonucleotide or oligonucleotide analogue. Oftentimes it results in less than 4 percent or 3 percent degradation of the compound. In certain cases it results in less than 2 percent or 1 percent of the compound.

The present invention is further directed to an instrument for polymer (e.g., DNA oligonucleotide) synthesis. For a discussion of DNA synthesizers, see: U.S. Pat. No. 5,368,823; U.S. Pat. No. 5,472,672; U.S. Pat. No. 5,529,756; U.S. Pat. No. 5,837,858. The preceding references are hereby incorporated-by-reference into this document for all purposes.

The instrument of the present invention typically includes one or more reservoirs containing chemical compounds used for synthesis of the subject polymer, where the reservoirs are operably connected in a system that allows flow of the various reagents (e.g., in a liquid medium) to a synthesis chamber (e.g., column including a solid support). There is a mechanism in the instrument to induce reagent flow (e.g., gas pressure) to the synthesis chamber, where the various chemical reactions involved in polymer synthesis are carried out. The synthesis chamber includes either an internal or external means to control its temperature (e.g., microwave device or heated jacket). The synthesized polymer exits the synthesis chamber through a valve that controls liquid flow. A computer controller is typically used to control flow of compounds from the reservoirs, the temperature of the synthesis chamber and exit of the polymer from the instrument.

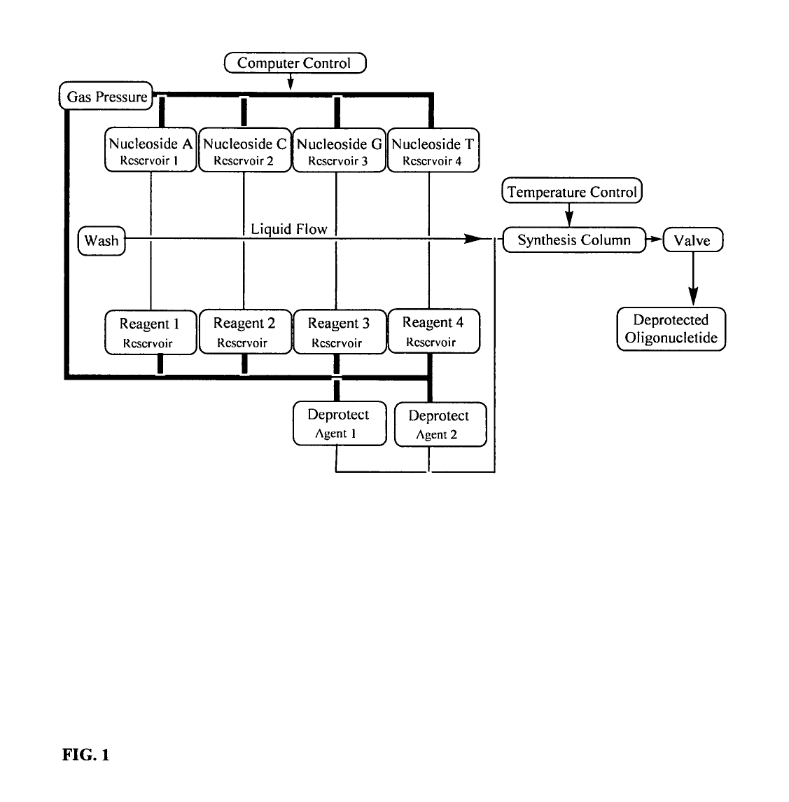

In reference to FIG. 1, a computer control unit controls gas pressure in a networked system of channels. The gas pressure directs flow of the various chemical compounds used in DNA oligonucleotide synthesis—“Nucleoside A, Reservoir 1”, “Nucleoside C, Reservoir 2”, “Nucleoside G, Reservoir 3”, “Nucleoside T, Reservoir 4”, “Wash”, “Reagent 1, Reservoir”, “Reagent 2, Reservoir”, “Reagent 3, Reservoir”, “Reagent 4, Reservoir”, “Deprotect, Agent 1”, and, “Deprotect, Agent 2”—to a synthesis column that includes a solid support. The exact order of chemical introduction into the synthesis column is dictated by the computer control unit to produce a protected, solid support bound DNA oligomer. A temperature control unit operably connected to the synthesis column adjusts the temperature of the column to facilitate, or effect, removal of one or more types of protecting groups from the oligonucleotide; in certain cases the temperature control unit further facilitates, or effects, removal of the oligonucleotide from the solid support to provide the oligonucleotide of interest.

In certain cases, the synthesis column and associated temperature controller are designed to effect removal of one or more BOC groups from an oligonucleotide under neutral conditions (i.e., pH of liquid medium used in oligonucleotide synthesis at approximately 7.0). This is done by heating the oligonucleotide attached to the solid support of the synthesis column to a temperature between 91° C. and 99° C. (or approximately 94° C.) for a period ranging from five minutes to 20 minutes.

In other cases, the synthesis column and associated temperature controller are designed to effect removal of an oligonucleotide from a solid support. This can occur where a tertiary alkyl group is used to link the oligonucleotide to the solid support, or where the tertiary alkyl group is part of a linker between the oligonucleotide and the solid support. As with BOC removal, cleavage of the oligomer from the solid support occurs under neutral conditions and involves heating of the solid support compound between 91° C. and 99° C. for a period ranging from five minutes to 20 minutes.

### Experimental Section

DNA syntheses were performed on a Biosearch 8750 synthesizer with Cruachem DNA amidites.

Anion exchange HPLC analyses were performed as follows: 2-20 mL of the aqueous samples, depending on the concentration, were injected onto a Dionex anion exchange column (4.6×250 mm); samples were eluted at 2 mL/min with aqueous buffers of (A) 0.025 M TRIS HCl and 0.01 M TRIS, and (B) 0.025 M TRIS HCl, 0.01 M TRIS and 1.0 M NaCl using a linear gradient of 1:0 to 0:1 over 20 min, with UV detection at 260 nm.

Samples for base composition analysis were treated as previously described¹⁶, with analysis by reverse phase HPLC as follows: 20 uL of the aqueous sample were injected onto a HAISIL HL C18 5 m column (4.6×150 mm); samples were eluted at 1 mL/min with buffers of (A) 0.1M TEAA, 5% acetonitrile, (B) acetonitrile, with a linear gradient of 1:0 to 0:1 over 20 min. UV detection at 260 nm.

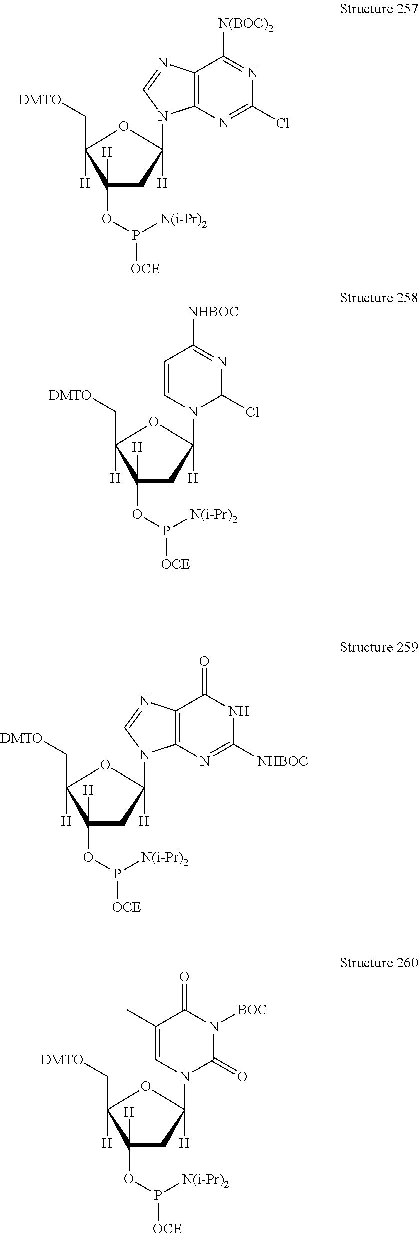

### 5′-O-(4,4′dimethoxtrityl)-N⁶,N⁶-(Di-tert-butyloxycarbonyl) deoxyadenosine-3′-O—N,N-diisopropyl cyanoethyl phosphoramidite (Structure 257)

To 5′-O-(4,4′dimethoxytrityl)-N⁶-(benzoyl)deoxyadenosine, (50 g, 76 mM) and imidazole, (20 g, 0.294 M), was added 700 mL dry pyridine and 50 g (0.333 M) tert-butyldimethylsilyl chloride. The solution was stirred for 18 hrs. The pyridine was removed by rotary evaporation and the residue was dissolved in 700 mL of ethyl acetate. The organic phase was washed with 500 ml of 0.5 M K₂HPO₄ followed by 500 mL saturated NaHCO₃. The solution was evaporated, giving 62 g of product 5′-O-(4,4′dimethoxytrityl)-N⁶-(benzoyl)-3′-O-tert-butyldimethylsilyl-deoxyadenosine. To a solution of this product in 900 mL of methanol was added 100 mL of conc. aqueous ammonia. After brief swirling, the solution was allowed to stand overnight. The solvents were removed by rotary evaporation, and the solid was re-dissolved in 700 mL of dry pyridine and 20 mL TEA, and 50 g di-tert-butyl pyrocarbonate was added. The solution was stirred for 18 hrs. The solvent was removed by rotory evaporation, the residue was dissolved in 500 mL DCM and this solution was washed with 500 mL 0.5 M KH₂PO₄. The organic phase was added to a silica column, 10×35 cm, packed with 2% methanol and 2% pyridine in DMF. A gradient to 6% methanol was applied to the column over 10 L of solvent, and fractions containing pure 5′-O-DMT-3′-O-tert-butyldimethylsilyl-N⁶,N⁶-(Di-tert-butyloxycarbonyl)-deoxyadenosine were pooled and reduced by rotary evaporation. The yield was 42 g (55 mM, 72% from DMT dA(Bz)).

The TBDMS group was removed by adding a solution of 60 mL 1 M TBAF in THF and 10 mL of HOAc in 500 mL of THF. After 18 hrs 50 mL saturated NaHCO₃ was added and the THF was removed by rotary evaporation. The residue was dissolved in 600 mL DCM and washed with 400 mL saturated NaHCO₃. The organic phase was added to a silica column, 10×35 cm, packed with 2% methanol and 2% pyridine in DCM. A gradient to 10% methanol was applied to the column over 14 L of solvent, and fractions containing pure 5′-O-DMT-N⁶,N⁶-(Di-tert-butyloxycarbonyl)-deoxyadenosine were pooled and reduced by rotary evaporation. The yield was 32 g (42.5 mM, 89% from 5′-O-DMT-3′-O-tert-butyldimethylsilyl-N⁶,N⁶-(Di-tert-butyloxycarbonyl)-deoxyadenosine). ¹H NMR (400 mHz, CDCl₃, PPM) 8.78 (s, 1H), 8.22 (s, 1H), 7.3-7.6 (m, 9H), 6.8 (d, 4H), 6.5 (t, 1H), 4.7 (s, 1H), 3.8 (s, 6H), 3.42 (d, 2H), 2.8 (m, 1H), 2.55 (m, 1H), 1.46 (s, 18H).

A solution of 2-cyanoethyl-N, N, N′,N′-tetraisopropylphosphorodiamidite (15 g, 50 mM) and 1H-tetrazole (1 g, 14 mM) in 800 mL dry acetonitrile was prepared and after 1 min of mixing this was added to the flask containing 32 g (49 mM) of dried 5′-O-DMT-N⁶,N⁶-(Di-tert-butyloxycarbonyl)-deoxyadenosine. The nucleoside slowly dissolved with swirling for 2 hr.

The solvent was removed by rotory evaporation and the residue was dissolved in EtOAc, 700 ml containing 300 mL of saturated NaHCO₃ solution. The mixture was shaken and allowed to separate, and the organic phase was added to a silica column, 10×25 cm, packed with 2% pyridine in EtOAc. The column was eluted isocratically, and fractions containing pure 257 were pooled and reduced by rotory evaporation to 33.9 g (35.6 mM, 84% yield from the protected nucleoside). ³¹P NMR (161 mHz, CDCl₃, PPM): 149.617, 149.410. Anal. Calc'd for C₅₀H₆₄N₇O₁₀P: C, 62.95. H, 6.76. N, 10.28. Found: C, 63.20. H, 6.79. N, 10.21.

### 5′-O-(4,4′dimethoxytrityl)-N⁴-(tert-butyloxycarbonyl) deoxycytosine-3′-O—N,N-diisopropyl cyanoethyl phosphoramidite (Structure 258)

To dry 5′-O-(4,4′dimethoxytrityl)-N⁴-(acetyl) deoxycytosine (50 g, 87.4 mM) was added imidazole (20 g, 0.294 M), 700 mL dry pyridine, and 50 g (0.333 M) tert-butyldimethylsilyl chloride. The solution was stirred for 18 hrs, the pyridine was removed by rotory evaporation, and the residue was dissolved in 700 mL of ethyl acetate. The organic phase was washed with 500 ml of 0.5 M K₂HPO₄ followed by 500 mL saturated NaHCO₃. Evaporation gave 56 g (81 mM) of 5′-O-(4,4′dimethoxytrityl)-N⁴-(acetyl)-3′-O-tert-butyldimethysilyl-deoxycytosine. A solution of this product in 900 mL of methanol was prepared, and to this was added 100 mL of conc. aqueous ammonia. After brief swirling, the solution was allowed to stand overnight. After drying, the solid was re-dissolved in 700 mL of dry pyridine and the solvent removed by rotory evaporation and high vacuum overnight. The solid was re-dissolved in 700 mL of dry THF and 20 g of dry K₂CO₃ were added. After 10 min 50 g (229 mM) di-tert-butyl pyrocarbonate was added. The solution was stirred for 18 hrs. The K₂CO₃ was removed by filtration and 200 mL of 0.5 MKH₂PO₄ was added. The THF was removed by evaporation. The residue was dissolved in 500 mL DCM and washed with 500 mL 0.5 M KH₂PO₄. The organic phase was added to a silica column, 10×35 cm, packed with 1% methanol and 1% pyridine in DCM. A gradient to 2% methanol was applied to the column after the first DMT containing bands eluted, and fractions containing pure 5′-O-DMT-3′-O-tert-butyldimethylsilyl-N⁴-(tert-butyloxycarbonyl)-deoxycytosine were pooled and reduced by rotary evaporation. The yield was 28 g (37.6 mM, 43% yield from DMT dC(Ac)). The TBDMS group was removed by adding a solution of 60 mL 1 M TBAF in THF and 10 mL of HOAc in 500 mL of THF. After 18 hrs, saturated NaHCO₃ (50 mL) was added and the THF was removed by rotary evaporation. The residue was dissolved in 600 mL DCM and washed with 400 mL saturated NaHCO₃. The organic phase was added to a silica column, 10×35 cm, packed with 2% methanol and 2% pyridine in DCM. A gradient to 10% methanol was applied to the column over 10 L of solvent, and fractions containing pure 5′-O-DMT-N⁴-(tert-butyloxycarbonyl)-deoxycytosine were pooled and reduced by rotory evaporation. The yield was 20 g (31.7 mM, 84% from 5′-O-DMT-3′-O-tert-butyldimethylsilyl-N⁴-(tert-butyloxycarbonyl)-deoxycytosine). ¹H NMR (400 mHz, CDCl₃, PPM): 8.2 (d, 1H), 7.3 (m, 9H), 7.0 (d, 1H), 6.85 (d, 4H), 6.3 (t, 1H), 4.5 (dd, 1H), 4.15 (dd, 1H), 3.8 (s, 6H), 3.5 (dd, 2H), 3.4 (dd, 1H), 2.75 (m, 1H), 2.35 (m, 1H), 1.5 (s, 18H).

A solution of 2-cyanoethyl-N, N, N′,N′-tetraisopropylphosphorodiamidite, 9 g (30 mM) and 1H-tetrazole, 650 mg (9 mM) in 500 mL dry acetonitrile was prepared and after 1 min of mixing this was added to the 20 g (31.7 mM) of dried 5′-O-DMT-N⁴-(-tert-butyloxycarbonyl)-deoxycytosine. The nucleoside slowly dissolved with swirling, and after 2 hrs the solvent was removed by rotory evaporation. The residue was dissolved in 500 ml EtOAc and shaken with 200 mL of saturated NaHCO₃ solution. The separated organic phase was added to a silica column, 5×25 cm, packed with 2% pyridine in EtOAc. The column was eluted isocratically, and fractions containing pure 258 were pooled and reduced by roary evaporation to 19.5 g (23.5 mM), 74% yield from the nucleoside. ³¹P NMR (161 mHz, CDCl₃, PPM): 149.997, 149.339. Anal. Calc'd for C₄₄H₅₆N₅₀O₉P: C, 63.68. H, 6.80. N, 8.44. Found: C, 63.59. H, 6.69. N, 8.52.

### 5′-O-(4,4′dimethoxvtrityl)-N²-(tert-butyloxycarbonyl) deoxyguanosine-3′-O—N,N-diisopropyl cyanoethyl phosphoramidite (Structure 259)

To dry 5′-O-(4,4′dimethoxytrityl)-N²-(isobutyryl)deoxyguanosine (100 g, 156 mM) was added imidazole (40 g, 0.588 M) in 1200 mL dry pyridine and 100 g (0.666 M) tert-butyldimethylsilyl chloride. The solution was stirred for 18 hrs, the pyridine was removed by rotary evaporation, and the residue was dissolved in 700 mL of ethyl acetate. The organic phase was washed with 500 ml of 0.5 M K₂HPO₄ followed by 500 mL saturated NaHCO₃. Drying produced 110 g (145 mM) of product 5′-O-(4,4′dimethoxytrityl)-N²-(isobutyryl)-3′-O-tert-butyldimethylsilyl-deoxyguanosine. A solution of the product in 1500 mL of methanol was prepared, and to this was added 150 mL of conc. aqueous ammonia. After brief swirling, the solution was allowed to stand overnight. The solvents were removed by rotary evaporation, and the dried solid was re-dissolved in 1400 mL of THF and 40 g of dry K₂CO₃ were added. After 10 min of stirring, 100 g di-tert-butyl pyrocarbonate was added. The solution was stirred for 3 hrs. TLC showed partial conversion (silica, 2% MeOH, 2% pyridine in DCM, rf starting material 0.3, rf product 0.7, visualized with 10% H₂SO₄ and heating). Longer reaction times gave less desired product and more side reaction materials. The K₂CO₃ was removed by filtration and 400 mL of 0.5 M KH₂PO₄ was added. The THF was removed by rotary evaporation and the residue was mixed with 700 mL DCM. The organic phase was added to a silica column, 10×35 cm, packed with 2% methanol and 2% pyridine in DMF. A gradient to 10% methanol was applied to the column over 14 L of solvent, and fractions containing pure 5′-O-DMT-3′-O-tert-butyldimethylsilyl-N²-(tert-butyloxycarbonyl)-deoxyguanosine were pooled and reduced by rotory evaporation. The yield was 25 g (34 mM, 22% yield from DMT dG(iBu)). The TBDMS group was removed by adding a solution of 60 mL 1 M TBAF in THF and 10 mL of HOAc in 500 mL of THF. After 18 hrs, saturated NaHCO₃, 50 mL, was added and the THF was removed by rotary evaporation. The residue was dissolved in 600 mL DCM and washed with 400 mL saturated NaHCO₃. The organic phase was reduced to a foam by rotary evaporation to give 5′-O-DMT-N²-(tert-butyloxycarbonyl)-deoxyguanosine (19.5 g, 29.1 mM, 86% from 5′-O-DMT-3′-O-tert-butyldimethylsilyl-N²-(tert-butyloxycarbonyl)-deoxyguanosine). The material was pure enough for the next step. An analytical sample was prepared by column chromatography as above on a silica column, 10×35 cM, packed with 2% methanol and 2% pyridine in DCM. A gradient to 10% methanol was applied to the column over 10 L of solvent, and fractions containing pure product were pooled and evaporated. ¹H NMR (400 mHz, CDCl₃, PPM) 7.7 (m, 1H), 7.2-7.4 (m, 10H), 6.8 (d, 4H), 6.2 (t, 1H), 5.7 (s, 2H), 4.65 (m, 1H), 4.15 (m, 1H), 3.8 (s, 6H), 3.35 (m, 2H), 2.7 (m, 1H), 2.45 (m, 1H), 1.6 (s, 18H).

A solution of 2-cyanoethyl-N, N, N′,N′-tetraisopropyl-phosphorodiamidite (12 g, 40 mM) and 1H-tetrazole (1 g, 14 mM) in 400 mL dry acetonitrile was prepared and after 1 min of mixing this was added to the flask containing 19.5 g (29.1 mM) of dried 5′-O-DMT-N²-(tert-butyloxycarbonyl)-deoxyguanosine. The nucleoside slowly dissolved with swirling, and after 2 hrs the solvent was removed by rotary evaporation and the residue was dissolved in EtOAc (500 ml) containing 100 mL of saturated NaHCO₃ solution. The mixture was shaken and allowed to separate, and the organic phase was added to a silica column, 6×25 cM, packed with 2% pyridine in EtOAc. The column was eluted isocratically, and fractions containing pure 259 were pooled and reduced by rotary evaporation to 12 g (14 mM, 45% yield from the nucleoside). ³¹P NMR (161 mHz, CDCl₃, PPM): 149.213, 149.175. Anal. Calc'd for C₄₅H₅₆N₇O₉P: C, 62.13. H, 6.49. N, 11.27. Found: C, 62.22. H, 6.69. N, 11.02.

### 5′-O-(4,4′dimethoxytrityl)-N³-(tert-butyloxycarbonyl)-thymidine-3′-O—N,N-diisopropyl cyanoethyl phosphoramidite (Structure 260)

5′-O-(4,4′dimethoxytrityl)-thymidine, 50 g (91.5 mM) was dried by rotary evaporation from 700 mL of dry pyridine and high vacuum overnight. Imidazole, 20 g (0.294 M) was added along with 700 mL dry pyridine and 50 g (0.333 M) tert-butyldimethylsilyl chloride. The solution was stirred for 18 hrs, whereupon TLC showed complete conversion (silica, 10% MeOH, 2% pyridine in DCM, rf starting material 0.5, rf product 0.9) visualized with 10% H₂SO₄ and heating). The pyridine was removed by rotary evaporation, and the product was dissolved in DCM, 700 mL. The solution was washed with 500 mL 0.5 M KH₂PO₄ followed by 500 mL sat'd aq. NaHCO₃. The solution was evaporated and subjected to high vacuum overnight. The yield was 60 g, 90.8%. 50 g of this product was dissolved in 1 L of THF and 25 g of anhydrous K₂CO₃ was added under Argon. The mixture was stirred for 30 min, and 50 g of ditertbutylpyrocarbonate was added. After this was completely dissolved, 12 g of DMAP was added. After overnight stirring, TLC revealed complete reaction (1:1 pet. ether:ethyl acetate, 2% pyridine rf starting material 0.4, rf product 0.8). The THF was removed by rotary evaporation and the residue was dissolved in 700 mL of ethyl acetate. The organic phase was washed with 500 ml of 0.5 M K₂HPO₄ followed by 500 mL sat'd NaHCO₃. The organic phase was added to a silica column, 10×35 cM, packed with 49% ethyl acetate, 49% pet. ether and 2% pyridine. The column was eluted isocratically, and fractions containing pure 5′-O-DMT-3′-O-tert-butyldimethylsilyl-N³-(tert-butyloxycarbonyl)-thymidine were pooled and reduced by rotory evaporation. The yield was 49.3 g, 86% yield. The TBDMS group was removed by adding a solution of 60 mL 1 M TBAF in THF and 10 mL of HOAc in 500 mL of THF. After 18 hrs, TLC showed complete conversion (silica, 2% MeOH, 2% pyridine in DCM, rf starting material 0.60, rf product 0.2, visualized with 10% H₂SO₄ and heating). Sat'd NaHCO₃, 50 mL, was added and the THF was removed by rotary evaporation. The residue was dissolved in 600 mL EtOAc and washed with 400 mL of water followed by 400 mL sat'd NaHCO₃. The organic phase reduced to a tar by rotary evaporation, then re-dissolved in 200 mL of DCM and added to a silica column, 10×35 cM, packed with 2% methanol and 2% pyridine in DCM. A gradient to 10% methanol was applied to the column over 10 L of solvent, and fractions containing pure 5′-O-DMT-N³-(tert-butyloxycarbonyl)-thymidine were pooled and reduced by rotary evaporation. The yield was 35 g (54.3 mM), 84% from 5′-O-DMT-3′-O-tert-butyldimethylsilyl-N³-(tert-butyloxycarbonyl)-thymidine. ¹H NMR (400 mHz, CDCl₃, PPM): 8.6 (d, 2H), 7.4-7.2 (m, 9H), 6.7 (dd, 4H), 6.37 (t, 1H), 4.6 (dd, 1H), 4.15 (dd, 1H), 3.8 (s, 6H), 3.5 (dd, 1H), 3.4 (dd, 1H), 2.7 (d, 1H), 2.35 (m, 2H), 1.6 (s, 9H), 1.45 (s, 3H). 25 g (45 mM) of the product was dried by solution in dry pyridine, 500 mL, and the solvent was removed by rotary evaporation followed by high vacuum overnight. A solution of 2-cyanoethyl-N, N, N′,N′-tetraisopropylphosphorodiamidite, 15 g (50 mM) and 1H-tetrazole, 650 mg (9 mM) in 500 mL dry acetonitrile was prepared and after 1 min of mixing this was added to the flask containing 25 g (45 mM) of dried 5′-O-DMT-N³-(-tert-butyloxycarbonyl)-thymidine. The nucleoside slowly dissolved with swirling, and after 2 hrs TLC showed complete conversion (silica, 2% pyridine in EtOAc, rf starting material 0.30, rf product 0.75 as two diasteromeric spots, visualized with 0.5% AgNO₃ and heat). The solvent was removed by rotary evaporation and the residue was dissolved in EtOAc, 500 ml containing 200 mL of sat'd NaHCO₃ solution.

The mixture was shaken and allowed to separate, and the organic phase was added to a silica column, 5×25 cM, packed with 49% ethyl acetate, 49% pet. Ether and 2% pyridine. The column was eluted isocratically, and fractions containing pure 260 were pooled and reduced by rotary evaporation to 25.5 g (29.6 mM), 76.3% yield from the nucleoside. ³¹P NMR (161 mHz, CDCl₃, PPM): 149.711, 149.105. Anal. Calc'd for C₄₅H₅₇N₄O₁₀P: C, 63.97. H, 6.80. N, 6.63.

Found: C, 63.74. H, 6.64. N, 6.78.

### General Synthesis of Boc Protected Ribonucleoside Phosphoramidites.

These reagents for the synthesis of RNA were prepared by treatment of the commercial base-protected 5′-O-DMT-2′-O-TBDMS ribonucleoside 3′-phosphoramidites with ammonia to remove the protecting group on the nucleobase. Treatment of this with di-tert-butyl pyrocarbonate gave the desired Boc-protected reagents for synthesis of RNA.

For example, the preparation of the riboC reagent.

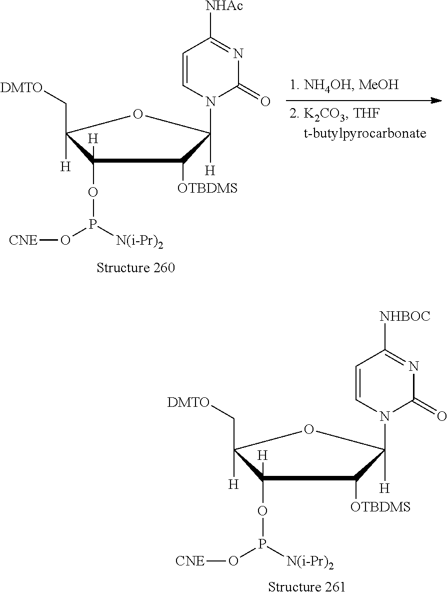

5′-O-DMT-2′-O-TBDMS-N⁴-acetylcytosine 3′-O—(N,N-diisopropyl cyanoethyl phosphor-amidite) was N-deprotected with aqueous ammonia in methanol. The product was dried well and treated with di-tert-butyl pyrocarbonate and potassium carbonate in THF. The product Boc RNA amidite was isolated by column chromatography. Likewise were prepared the fully protected N⁶-diBoc-adenosine, N²—Boc-guanosine, and N-1-Boc-uridine phosphoramidites.

### General Synthesis of CPG-Boc-Nucleoside

The 5′-DMT-N-(Boc) nucleoside was treated with diglycolic anhydride and catalytic N-methylimidazole in dry pyridine, and the resulting 3′-ester purified by column chromatography. 10 g of 1000 A aminopropyl CPG was treated with 400 mg of the glycolate, 400 mg BOP and 400 microliters N-methylmorpholine in acetonitrile sufficient to form a thick slurry with the CPG. Standing overnight, followed by washing, capping and drying gave the derivatized CPG at a loading of 30 micromoles/g.

### Time Course for Deprotection of a Single Boc Residue on a T₁₀ Oligonucleotide

### Preparation and Deprotection of Boc-dC-T10

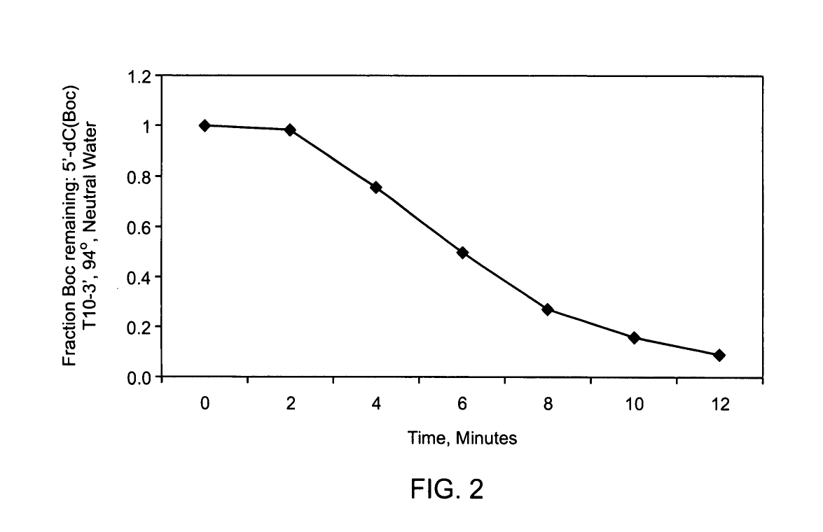

The dC amidite was coupled to T-10, the DNA was cleaved and deprotected with 25% 2-methoxyethylamine in methanol (1 mL) for 3 hrs at room temp, remove CPG and evaporate to dryness, re-dissolve in DI water (1 mL). ESMS showed the correct mass of the DNA with the t-butyl group (M+100) still attached. RP HPLC was used to follow the heat induced N-4-Boc-dC deprotection; the Boc protected dC-T-10 had a longer retention time with baseline separation from the oligo without it. Integration gave relative amounts of each species in solution. A 3 hr time course showed complete deprotection in 15 min; a 12 min time course (shown in FIG. 2) showed orderly deprotection with T ½ about 6 min.

Deprotection of (Boc)₂-dA-T₁₀

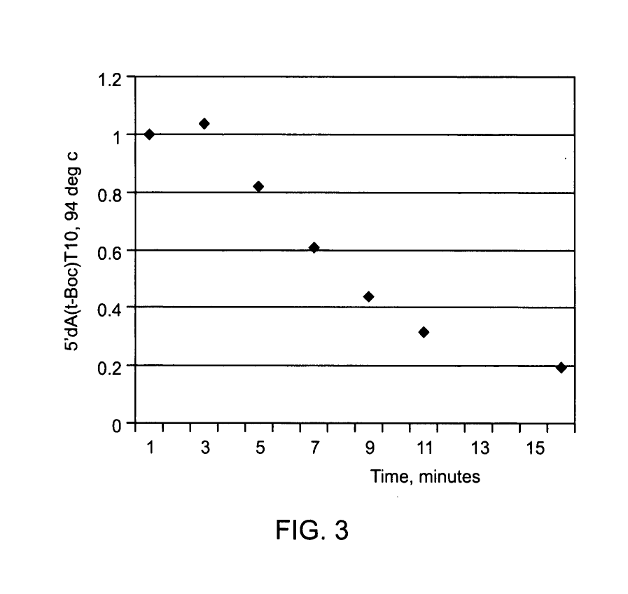

The BisBoc-dA amidite was coupled to T-10, the DNA was cleaved and deprotected with 25% 2-methoxyethylamine in methanol (1 mL) for 3 hrs at room temp, remove CPG and evaporate to dryness, re-dissolve in DI water (1 mL). ESMS showed the correct mass of the DNA with the t-butyl group (M+100) still attached. RP HPLC was used to follow the heat induced Boc deprotection. A 15 minute time course showed 80% deprotection in 15 min with Ty. of about 7 min. The NMR and ESMS data show that one of the t-Butyl carbonates is removed by the base treatment during removal of the oligonucleotides from the CPG, leaving a single Boc on adenine residues. See FIG. 3.

### Boc-Protected RNA

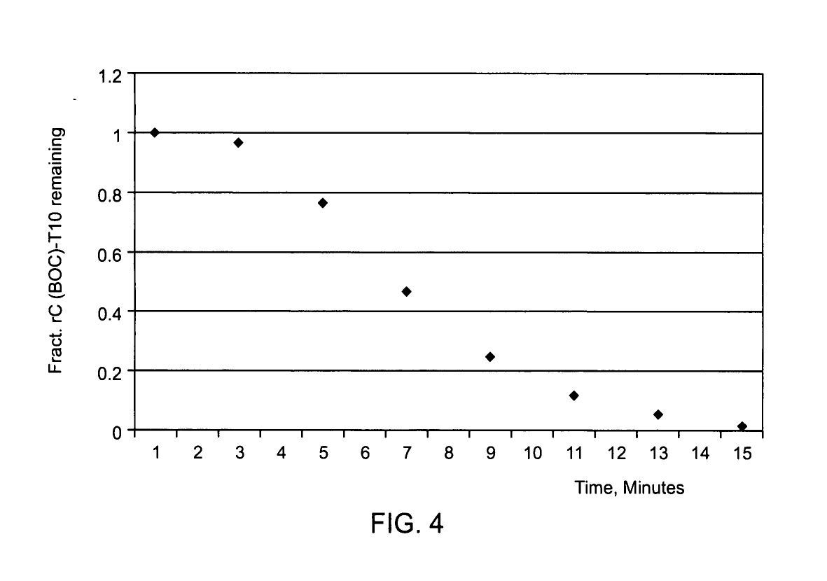

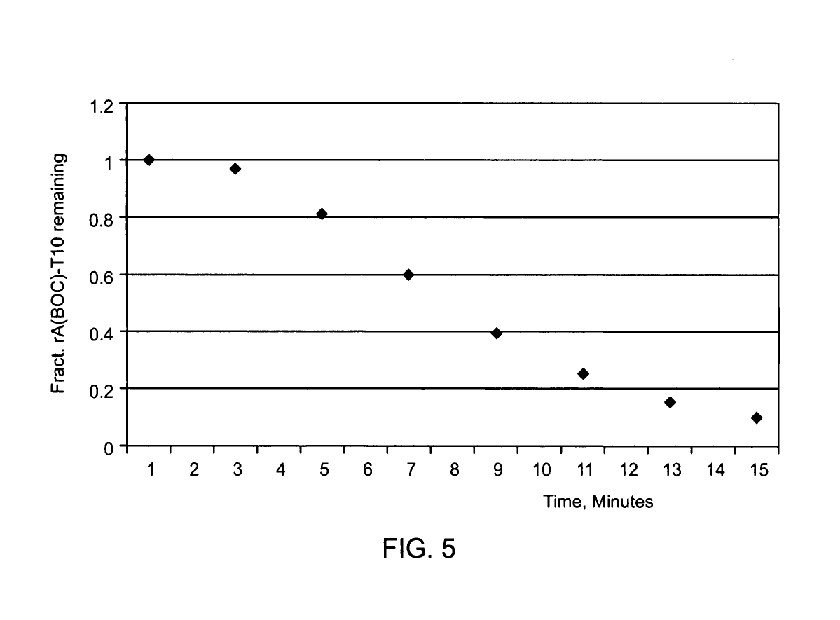

The ribonucleoside amidites coupled to T₁₀ with at least 90% efficiency with 10-15 min coupling times. Each one was coupled to T-10 and inspected by RP HPLC. They were 98% pure by phosphorus NMR. When a sample was heated for 1 hr at 94 deg. C., complete removal of the Boc group was observed on heating in all cases. The results were confirmed by ESMS. Time courses to measure the rate of the conversion, using HPLC for easy assessment of the amounts of each species present. The results for the rC- and rA-T10 oligonucleotides are shown in FIGS. 4 and 5:

### Deprotection of a Boc-Protected PCR Primer

The RNaseP forward primer 19-mer [AGATTTGGACCTGCGAGCG](SEQ ID NO: 1) was synthesized on Boc-dG CPG (1 μmol) with Boc dA, dC, and dG amidites. The fully deprotected mass for this primer is 5868 g/mol and the expected mass for Boc protection is 7369 g/mol [15 Boc residues.100 amu/Boc=1500 added]. Deprotection of side chain protecting groups was performed with 25% 2-methoxyethylamine in methanol (1 mL) for 3 hrs at room temp, remove CPG and evaporate to dryness, re-dissolve in DI water (1 mL). The DMT was removed before cleavage because the Boc group can be used as a hydrophobic handle. The product was desalted with 20-50 micron polystyrene beads packed in a 1 mL cartridge and eluted in 20% ACN/H₂O.

The desalted Boc-primer was aliquoted into three microfuge tubes of 100 uL each. The samples were dried down and brought up in 1 mL of house DI water. Only water was added to the first tube. To the second tube 1 mL of PCR buffer pH 8.5, 1× with MgCl₂, was added to a final concentration of 6 mM (standard PCR concentrations). To the third tube TEAA was added to give a final concentration of 0.025 N. Each of the three samples was aliquoted into ten 200 uL thin-walled PCR tubes for a total of 30 tubes. All primer preparation was done at room temperature.

An ABI 9700 Thermocycler was preheated to 94° C. Each sample was placed into the heat block and removed at the time point and placed on dry ice to stop the reaction. As a negative control, the T=0 sample was not heated and placed directly in ice at the beginning of the experiment. The samples were allowed to thaw at room temperature and transferred to a 96-well plate for HPLC and mass spectroscopy analysis.

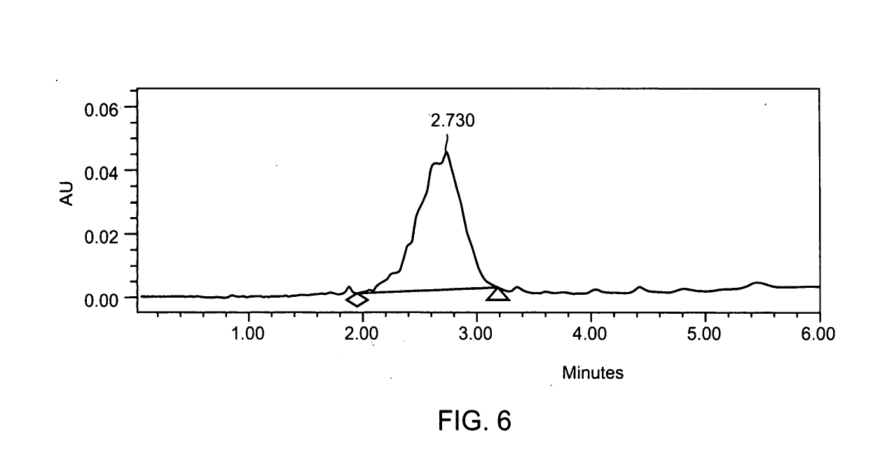

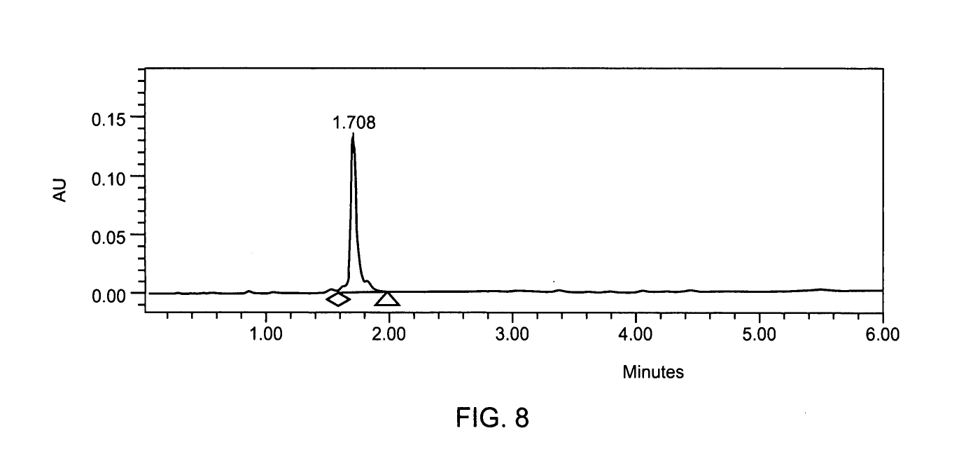

For each time point, reverse-phase and mass spec data were acquired to track the removal of the Boc protecting group. The Boc protected primer is not a single species but rather a collection at various stages of protection. In the T=0 sample, a series of species differing by 100 mass units shows that some of the Boc groups have already come off, probably by exposure to the high temperature inlet port of the mass spectrometer. See HPLC shown in FIG. 6 and ESMS shown in FIG. 7.

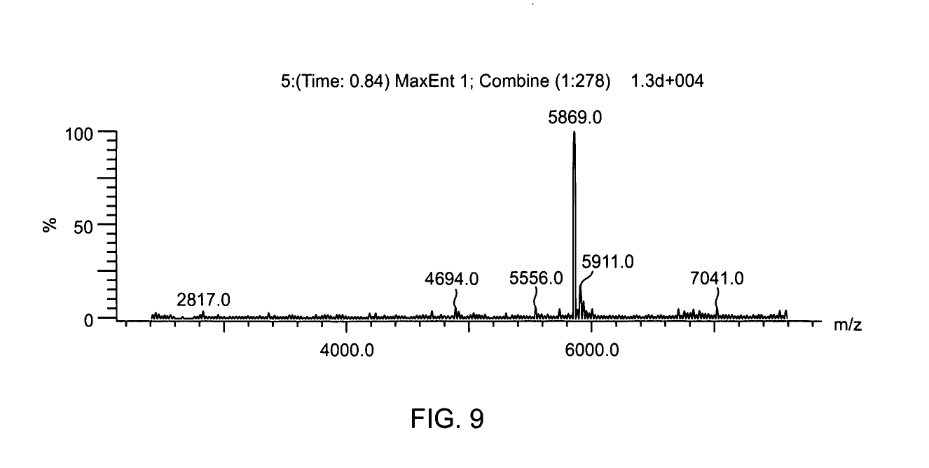

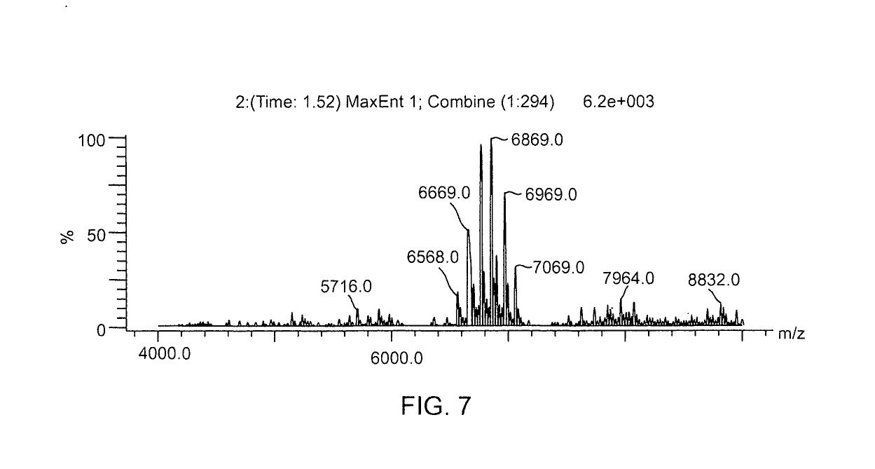

After 10 minutes almost all of the Boc groups were gone; however some remained in the TEAA samples. After 15 minutes in TEAA, all were removed. Importantly, the primer is deprotected after 10 minutes under normal PCR buffering. See HPLC shown in FIG. 8 and ESMS shown in FIG. 9.

1. A compound of the structure XO—CH₂—SM-B-A, wherein X is H, an acid labile protecting group, a solid support, —P(O—R′)NR²R³, —P(O)(OH)H, —P(O)(OR′)H, —P(O)(OH)₂, —P(O)(OH)O—P(O)(OH)OP(O)(OH)₂ or salts thereof, wherein R¹ is alkyl, substituted alkyl, heteroalkyl, substituted heteroalkyl, aryl or substituted aryl, and R² and R³ are independently alkyl, substituted alkyl, heteroalkyl, substituted heteroalkyl, aryl or substituted aryl, or R² and R³ combine to form a cyclic, fused, fused cyclic or heterocyclic ring, SM is a sugar moiety or analogue thereof that is not a natural furanosyl, B is a base moiety or analogue thereof, and A is a moiety attached to a nitrogen on or in the base moiety of the structure —C(O)OR⁴, wherein R⁴ is tertiary alkyl.

2. The compound according to claim 1, wherein SM is selected from the following moieties, where “X” is H, a protecting group, a solid support which optionally includes a linker between the oxygen and the solid support, a phosphorus containing moiety or salts thereof; “B” is a nucleobase moiety or an analogue of a nucleobase moiety; “A” is one or more moieties attached to one or more nitrogen atoms on or within the base moiety and is of the structure —C(O)OR¹, where R¹ is a tertiary alkyl group; “X¹” is H, a protecting group, a solid support which optionally includes a linker between the oxygen and the solid support, a phosphorus containing moiety or salts thereof; Y is OH or OR² where R² is a protecting group, an alkyl, a substituted alkyl, a heteroalkyl, a substituted heteroalkyl, an aryl or a substituted aryl; Z is H, OH or OR³ where R³ is a protecting group, an alkyl, a substituted alkyl, a heteroalkyl, a substituted heteroalkyl, an aryl or a substituted aryl; R⁴ and R⁵ are, independently, H, alkyl, substituted alkyl, heteroalkyl, substituted heteroalkyl, aryl, or substituted aryl; “m” and “o” and independently 0, 1 or 2; “R” is alkyl, substituted alkyl, aryl or substituted aryl; R⁶ is H, alkyl, substituted alkyl, heteroalkyl, substituted heteroalkyl, aryl or substituted aryl; R⁷ is OH, a halide, OR⁸, NR⁹R¹⁰, where R⁸ is alkyl, substituted alkyl, aryl, heteroalkyl, substituted heteroalkyl, aryl, or substituted aryl, and where R⁹ and R¹⁰ are independently H, alkyl, substituted alkyl, aryl, heteroalkyl, substituted heteroalkyl, aryl, or substituted aryl:

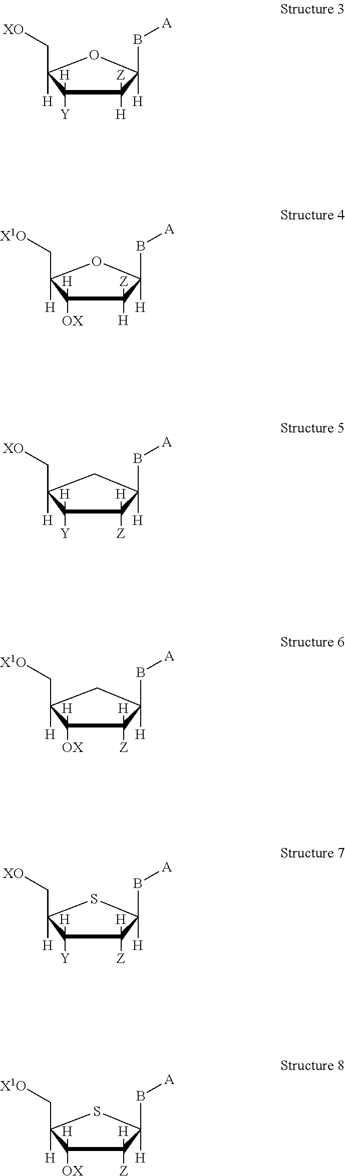

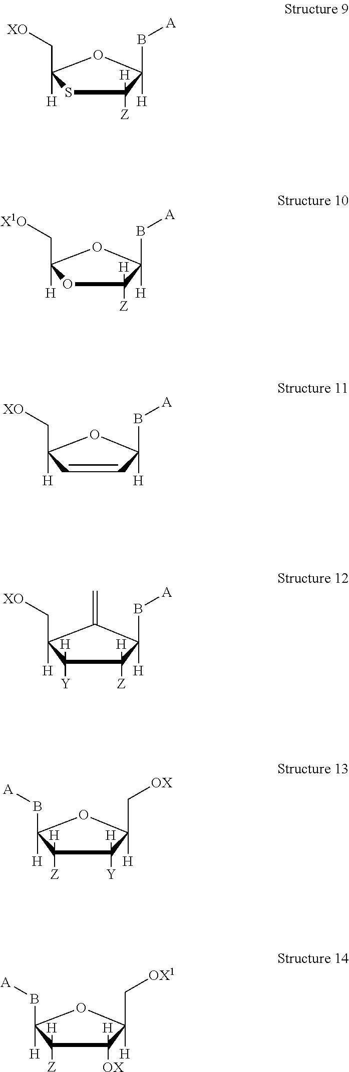

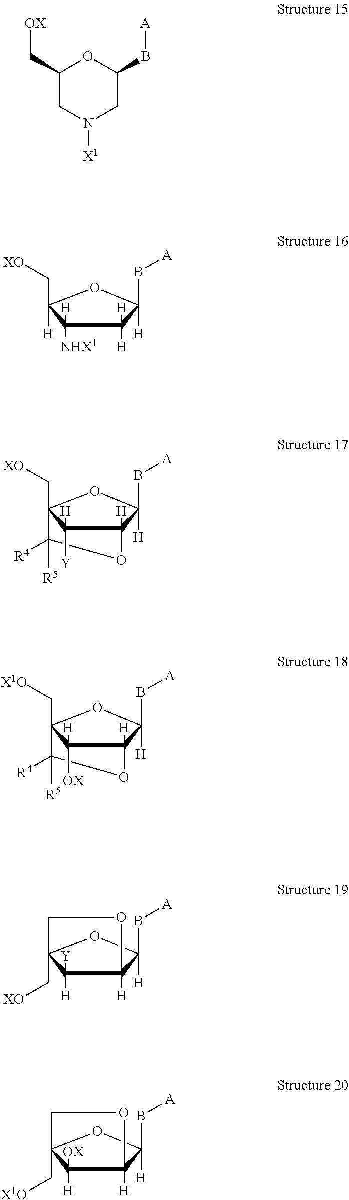

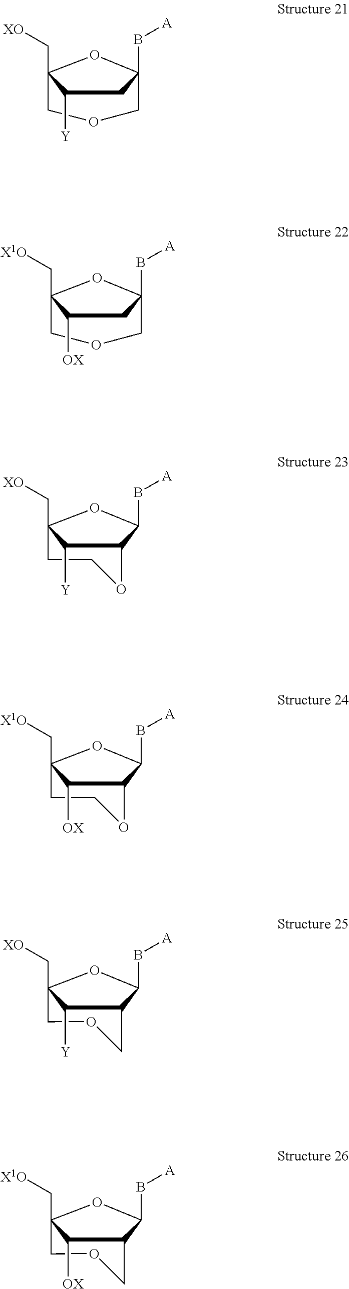

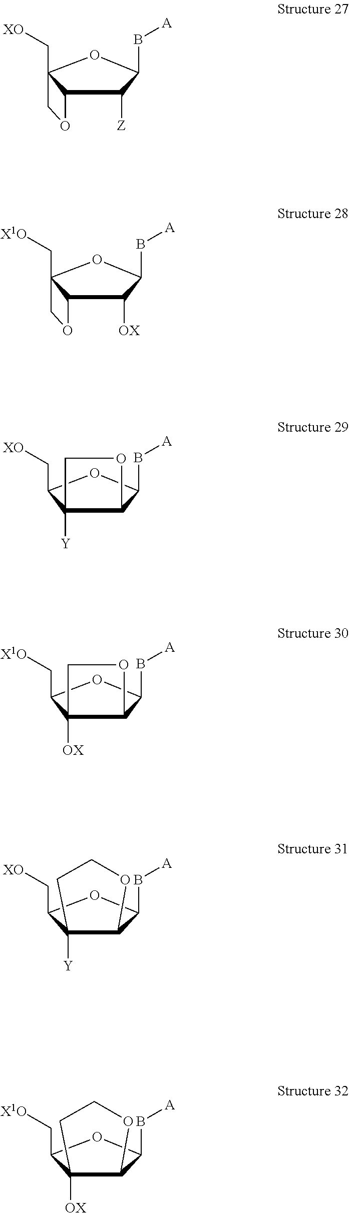

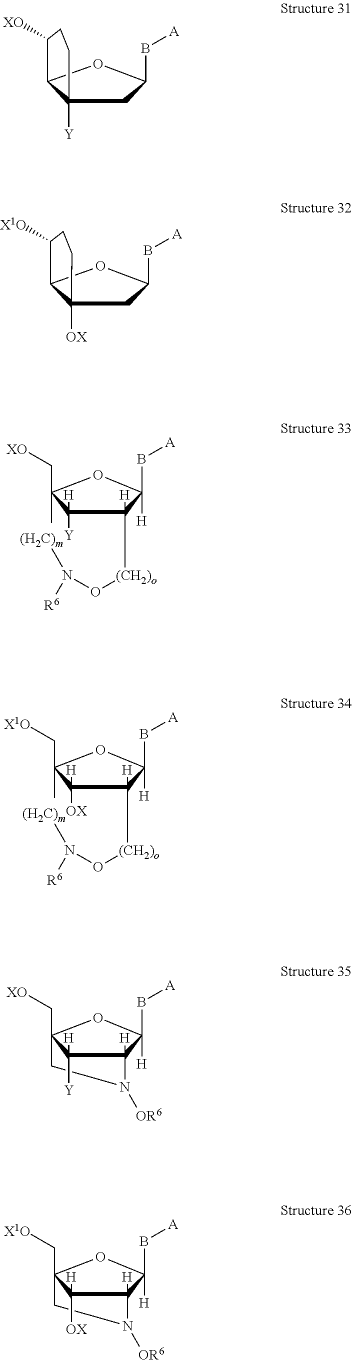

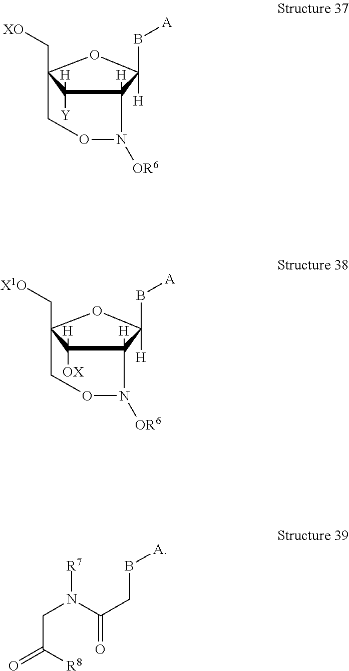

3. The compound according to claim 1, wherein “B” is selected from the following moieties, where “A” is of the structure —C(O)OR¹, where R¹ is a tertiary alkyl group; where “M” is N or CR³, where R³ is H, halo, alkyl, substituted alkyl, heteroalkyl, substituted heteroalkyl, phenyl, substituted phenyl, alkenyl, alkynyl, OH, SH, or NR⁴R⁵, where R⁴ and R⁵ are, independently H or alkyl; and where R¹² is H, halo, alkyl, substituted alkyl, heteroalkyl, substituted heteroalkyl, aryl, substituted aryl, alkenyl, alkynyl, OH, SH, or NR⁴R⁵, where R⁴ and R⁵ are, independently H or alkyl; and, where “D” and “E” are independently N or CR³, where R³ is H, halo, alkyl, substituted alkyl, heteroalkyl, substituted heteroalkyl, aryl, substituted aryl, alkenyl, alkynyl, OH, SH, or NR⁴R⁵, where R⁴ and R⁵ are, independently H or alkyl:

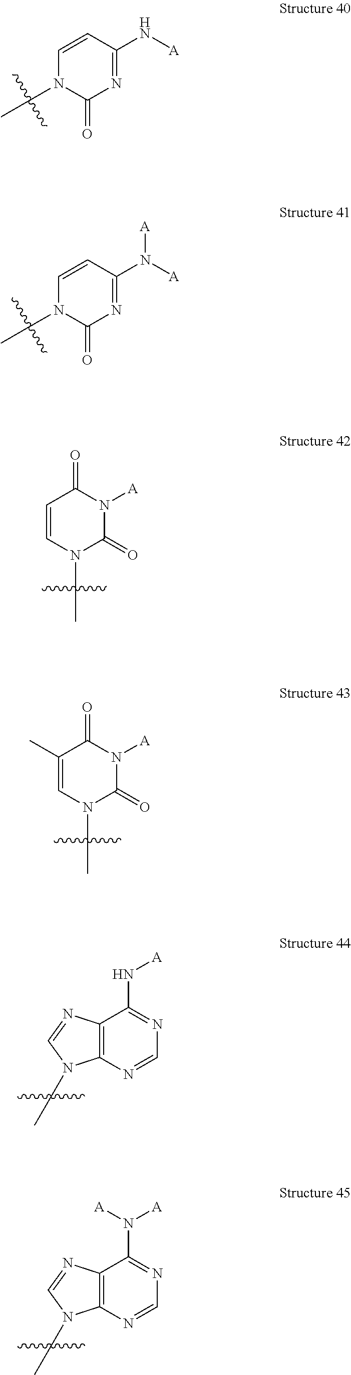

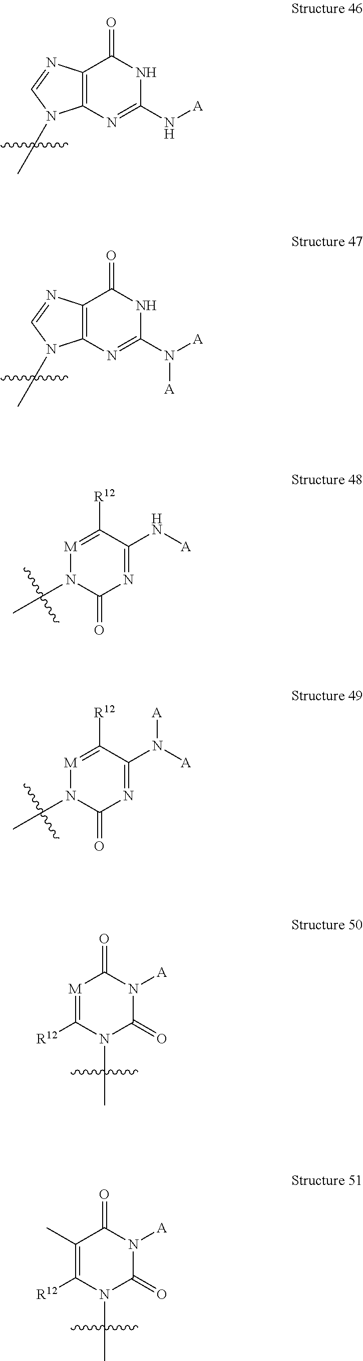

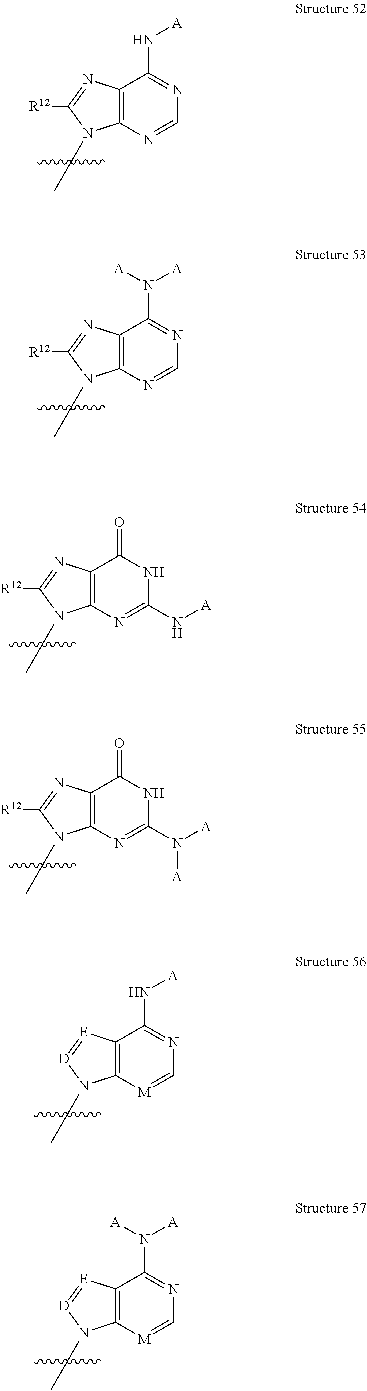

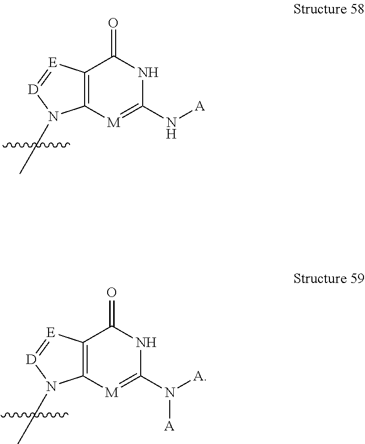

4. The compound according to claim 1, wherein “A” is a moiety selected from the following moieties: —C(CH₃)₃; —C(CH₃)₂(CH₂CH₃); —C(CH₃)(CH₂CH₃)(CH₂CH₂CH₃); —C(R¹⁴)(R¹⁵)-Linker-Label; and —C(R¹⁴)(R¹⁵)-Linker-[Solid Support], wherein R¹⁴ and R¹⁵ are independently selected from —CH₃, —CH₂CH₃, —CH₂CH₂CH₃, and CH(CH₃)₂.

5. The compound according to claim 1, wherein X is H, —P(O—R′)NR²R³, —P(O)(OH)O—P(O)(OH)OP(O)(OH)₂ or salts thereof, wherein R¹ is alkyl, substituted alkyl, aryl or substituted aryl, and R² and R³ are independently alkyl, substituted alkyl, aryl or substituted aryl, or R² and R³ combine to form a cyclic, fused, fused cyclic or heterocyclic ring.

6. A compound of the structure XO—CH₂—SM-B-A, wherein X is H, an acid labile protecting group, a solid support, —P(O—R′)NR²R³, —P(O)(OH)H, —P(O)(OR′)H, —P(O)(OH)₂, —P(O)(OH)O—P(O)(OH)OP(O)(OH)₂ or salts thereof, wherein R¹ is alkyl, substituted alkyl, heteroalkyl, substituted heteroalkyl, aryl or substituted aryl, and R² and R³ are independently alkyl, substituted alkyl, heteroalkyl, substituted heteroalkyl, aryl or substituted aryl, or R² and R³ combine to form a cyclic, fused, fused cyclic or heterocyclic ring, SM is a sugar moiety or analogue thereof that is a furanosyl moiety of the structure 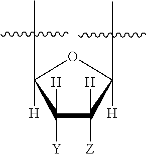
B is a nucleobase moiety or analogue thereof, and A is a moiety attached to a nitrogen on or in the base moiety of the structure —C(O)OR⁴, wherein R⁴ is tertiary alkyl;
wherein when Y is —OP(O-CNE)NR¹R² or —OP(O)(OH)H or salts thereof, then X is an acid labile protecting group or a solid support, Z is H or OR⁵, wherein R⁵ is a hydroxyl protecting group;
wherein when X is —P(O-CNE)(NR¹R²) or —P(O)(OR³)H or salts thereof, then Y is an acid labile hydroxyl protecting group or a solid support and Z is H; and,
wherein when X is —P(O)(OR³)H or —P(O)(OH)O[P(O)(O⁻)(O⁻)]ₙH or salts thereof wherein n=0, 1 or 2, then Y is OH or OR⁶ wherein R⁶ is a thermolabile hydroxyl protecting group, and Z is H, —OH, or OR⁶.

7. The compound according to claim 6, wherein “B” is selected from the following moieties, where “A” is of the structure —C(O)OR¹, where R¹ is a tertiary alkyl group; where “M” is N or CR³, where R³ is H, halo, alkyl, substituted alkyl, heteroalkyl, substituted heteroalkyl, phenyl, substituted phenyl, alkenyl, alkynyl, OH, SH, or NR⁴R⁵, where R⁴ and R⁵ are, independently H or alkyl; and where R¹² is H, halo, alkyl, substituted alkyl, heteroalkyl, substituted heteroalkyl, aryl, substituted aryl, alkenyl, alkynyl, OH, SH, or NR⁴R⁵, where R⁴ and R⁵ are, independently H or alkyl; and, where “D” and “E” are independently N or CR³, where R³ is H, halo, alkyl, substituted alkyl, heteroalkyl, substituted heteroalkyl, aryl, substituted aryl, alkenyl, alkynyl, OH, SH, or NR⁴R⁵, where R⁴ and R⁵ are, independently H or alkyl:

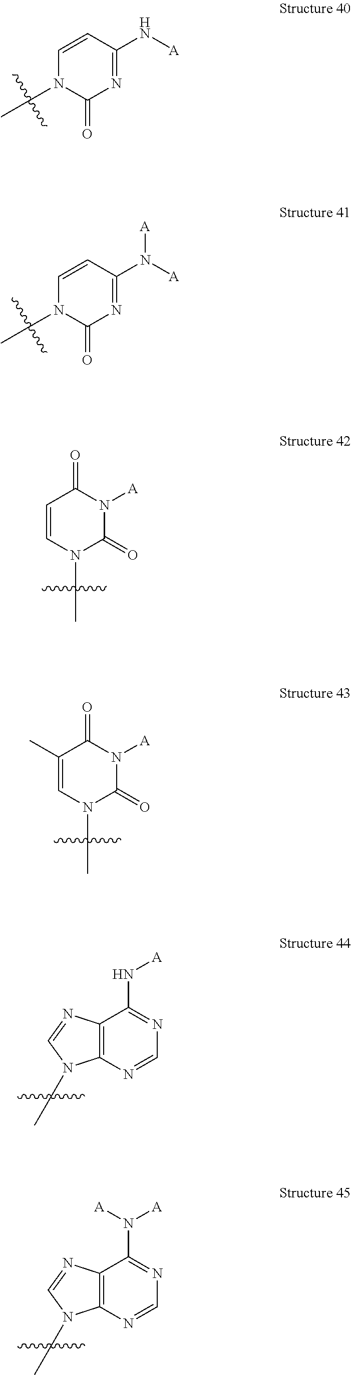

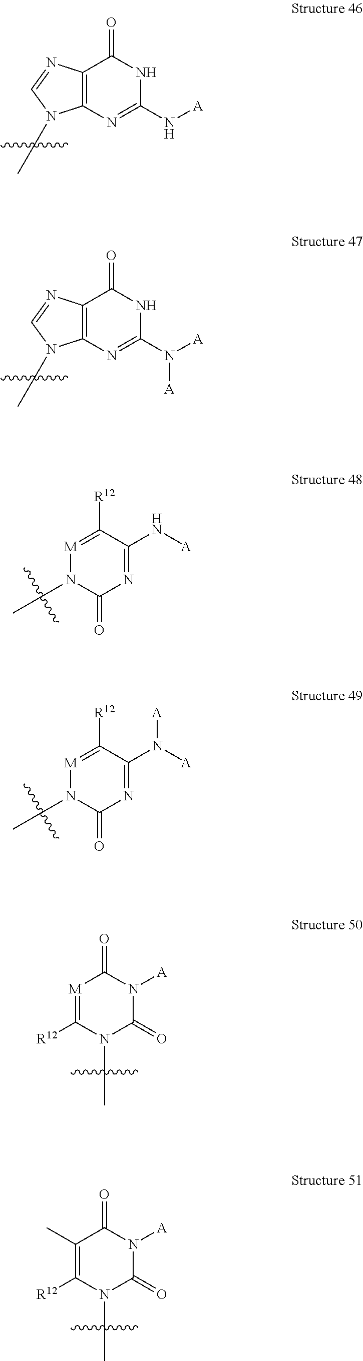

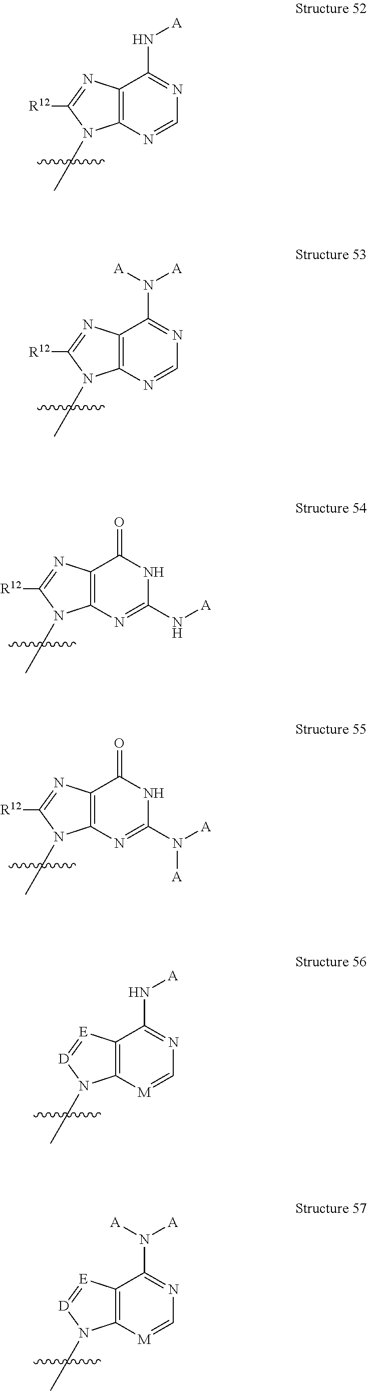

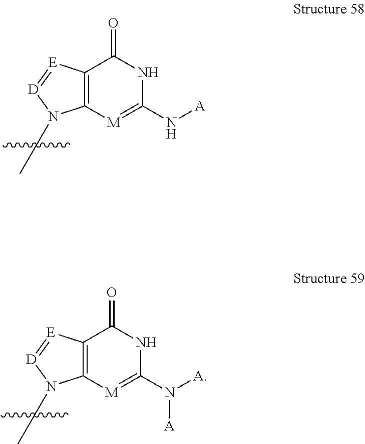

8. The compound according to claim 6, wherein “A” is a moiety selected from the following moieties: —C(CH₃)₃; —C(CH₃)₂(CH₂CH₃); —C(CH₃)(CH₂CH₃)(CH₂CH₂CH₃); —C(R¹⁴)(R¹⁵)-Linker-Label; and —C(R¹⁴)(R¹⁵)-Linker-[Solid Support], wherein R¹⁴ and R¹⁵ are independently selected from —CH₃, —CH₂CH₃, —CH₂CH₂CH₃, and CH(CH₃)₂.

9. The compound according to claim 6, wherein the compound is selected from the following group where “X” is —P(O)(OH)₂, —P(O)(OH)OP(O)(OH)₂, —P(O)(OH)OP(O)(OH)O—P(O)(OH)₂ or salts thereof, and where “Z” is —H or —OH:

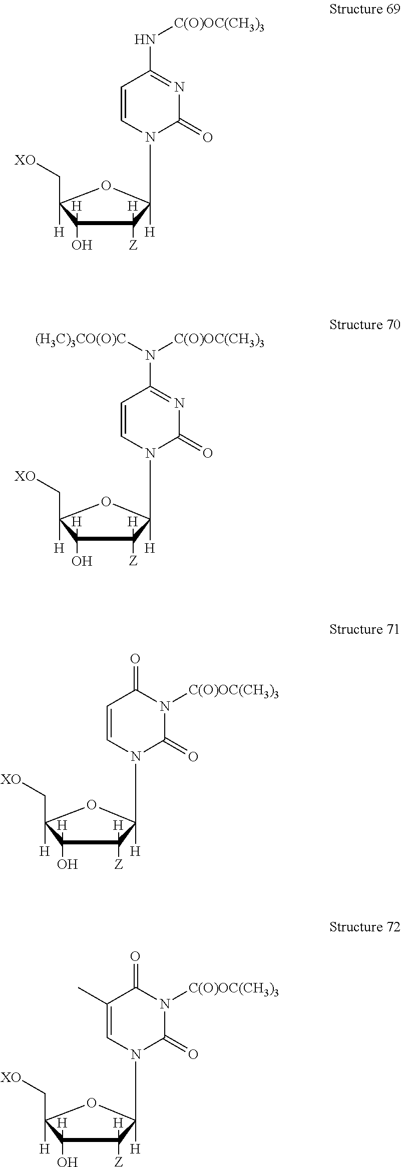

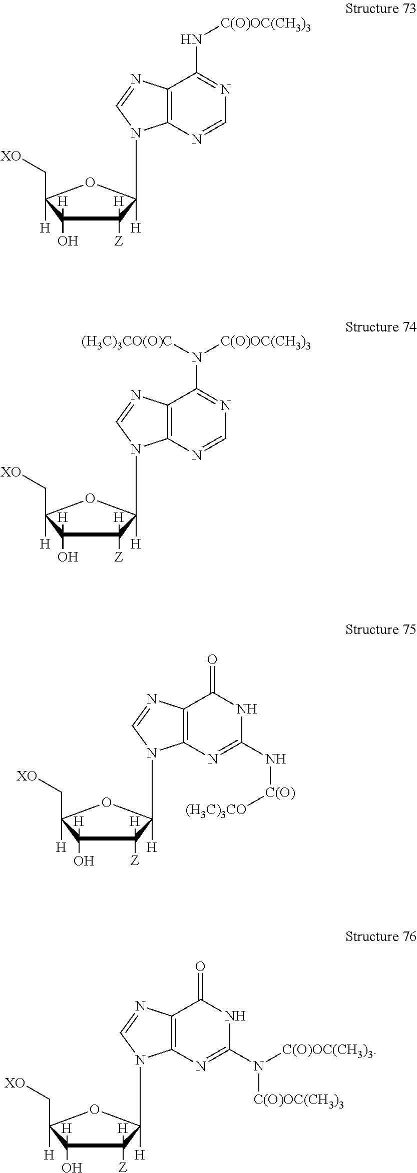

10. An oligonucleotide, wherein the oligonucleotide comprises one or more nucleosides or modified nucleoside analogues of the structure —O—CH₂—SM(—O—)B-A, wherein SM is a sugar moiety or analogue thereof, B is a nucleobase moiety or analogue thereof, and A is a moiety attached to a nitrogen on or in the base moiety of the structure —C(O)OR⁴, wherein R⁴ is tertiary alkyl.

11. The oligonucleotide according to claim 10, wherein the oligonucleotide is of the structure 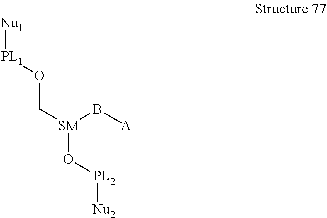
wherein PL₁ and PL₂ are, independently, either H or —P(O)(OH)O— or an analogue thereof, and Nu₁ and Nu₂ are, independently, no substituent, a nucleoside or nucleoside analogue, or an oligonucleotide.

12. The oligonucleotide according to claim 10, wherein the oligonucleotide is of one of the following structures: 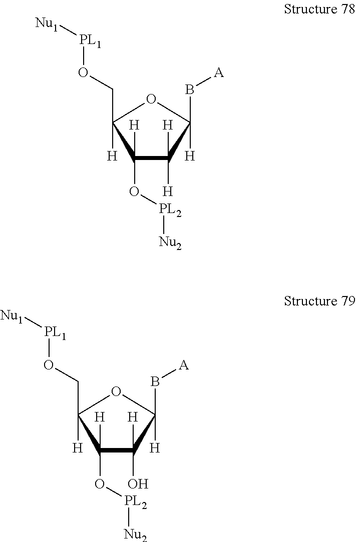
wherein PL₁ and PL₂ are, independently, either H or —P(O)(OH)O— or an analogue thereof, and Nu₁ and Nu₂ are, independently, no substituent, a nucleoside or nucleoside analogue, or an oligonucleotide.

13. The oligonucleotide according to claim 10, wherein the oligonucleotide is selected from the following group of oligonucleotides, wherein PL₁ and PL₂ are, independently, either H or —P(O)(OH)O— or an analogue thereof, and Nu₁ and Nu₂ are, independently, no substituent, a nucleoside or nucleoside analogue, or an oligonucleotide:

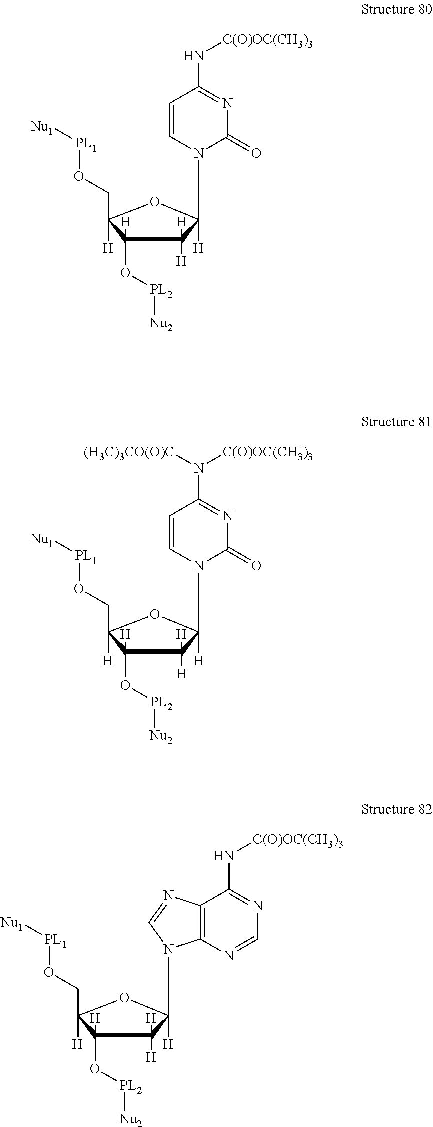

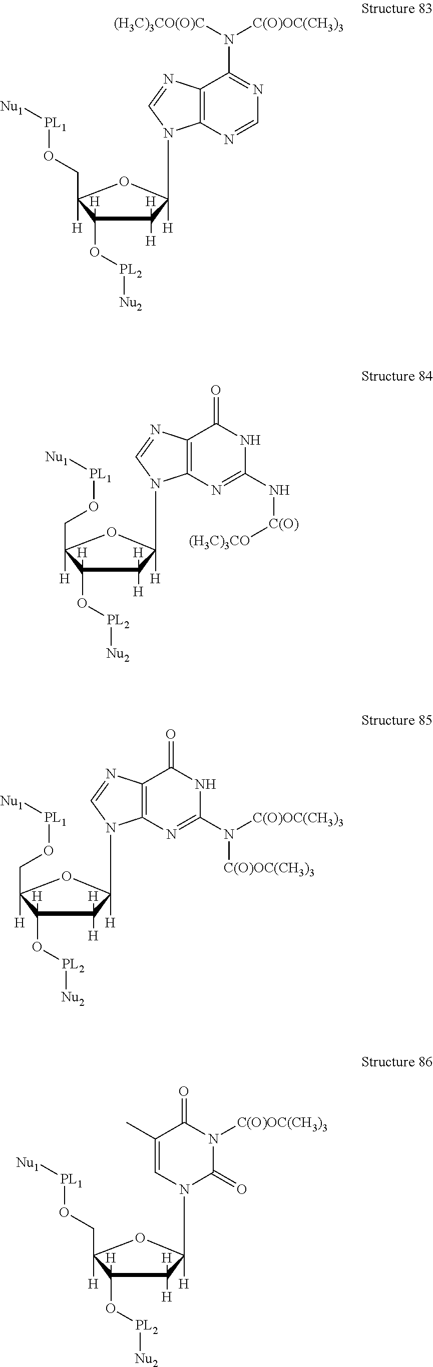

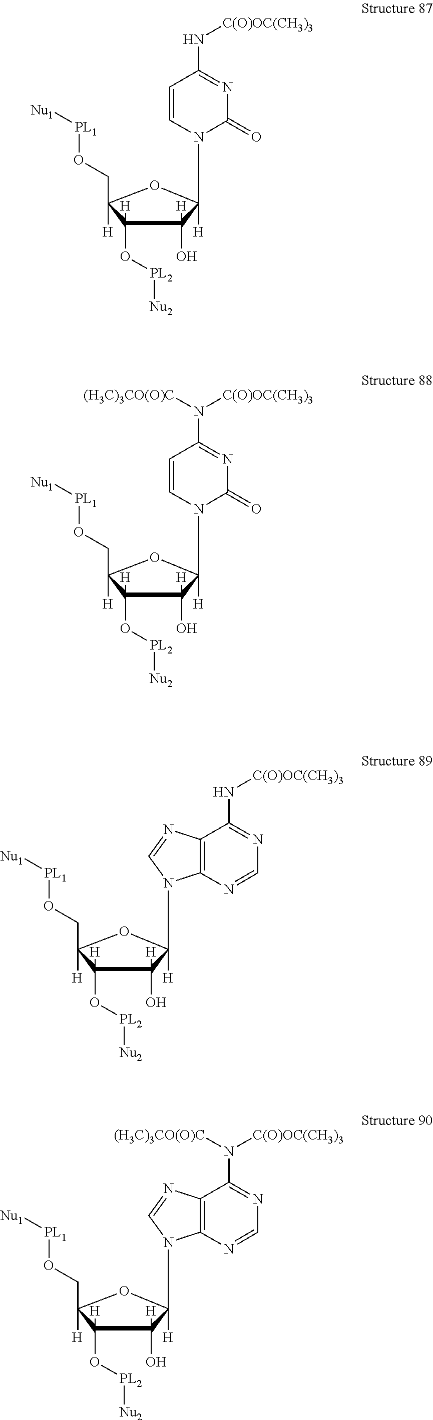

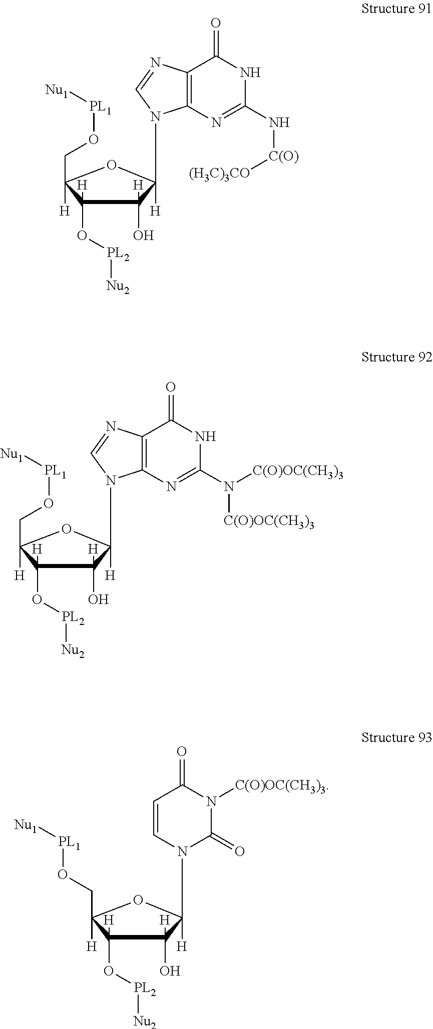

14. A therapeutically active nucleoside, a therapeutically active nucleoside analogue, a therapeutically active nucleotide, therapeutically active nucleotide analogue or therapeutic oligonucleotide, wherein at least one moiety of structure —C(O)OR⁴ is bound to a nucleobase on the nucleoside, nucleoside analogue, nucleotide, nucleotide analogue or oligonucleotide, and wherein R⁴ is tertiary alkyl.

15. The therapeutically active nucleoside, a therapeutically active nucleoside analogue, a therapeutically active nucleotide, therapeutically active nucleotide analogue or therapeutic oligonucleotide according to claim 14, where it is selected from the following group, where A₁, A₂ and A₃ are independently H or a thermally labile protecting group, and where at least one of the thermally labile protecting groups is of the structure —C(O)OR⁸, and where R⁸ is a tertiary alkyl group, and B is a nucleobase or nucleobase analogue:

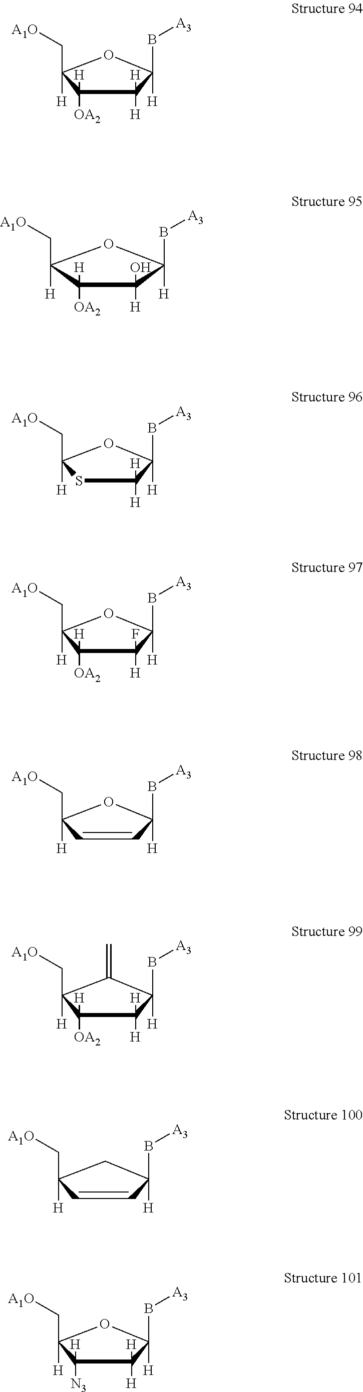

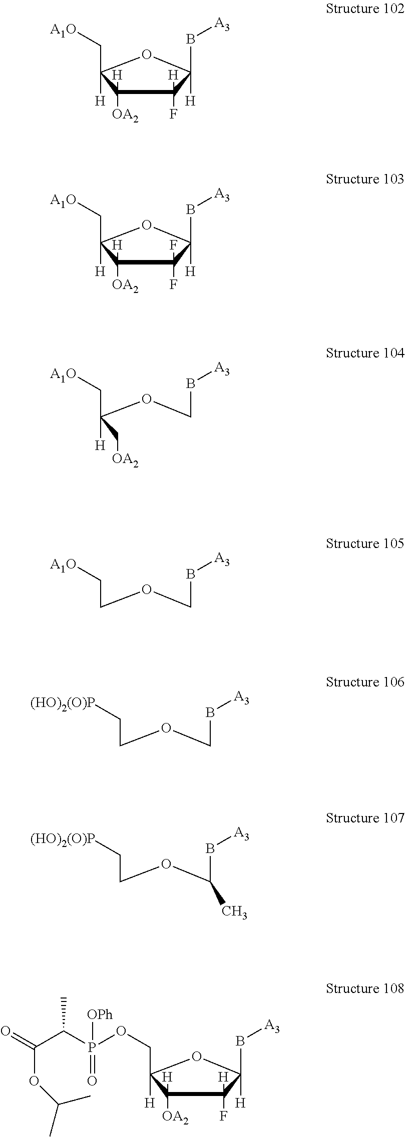

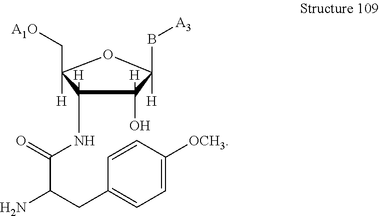

16. The therapeutically active nucleoside, a therapeutically active nucleoside analogue, a therapeutically active nucleotide, therapeutically active nucleotide analogue or therapeutic oligonucleotide according to claim 14, where it is selected from the following group:

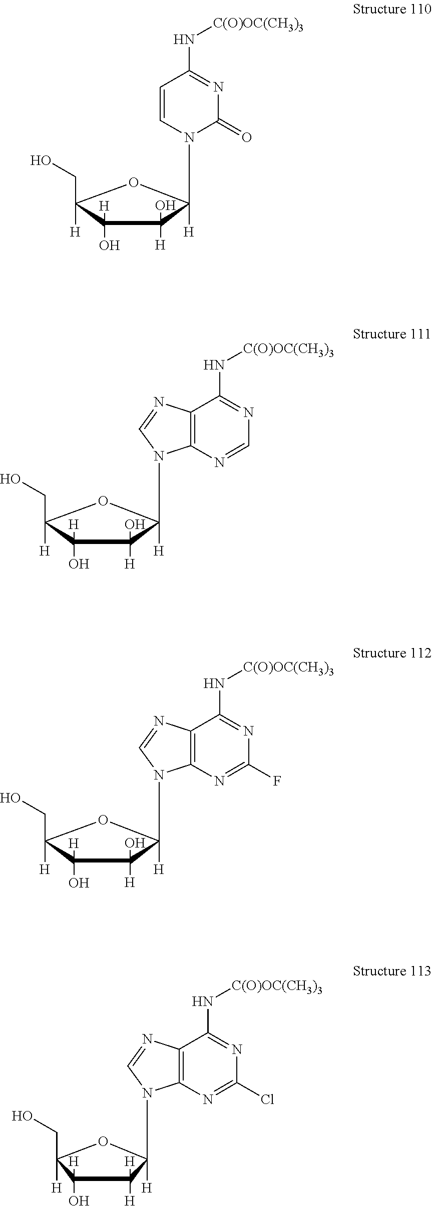

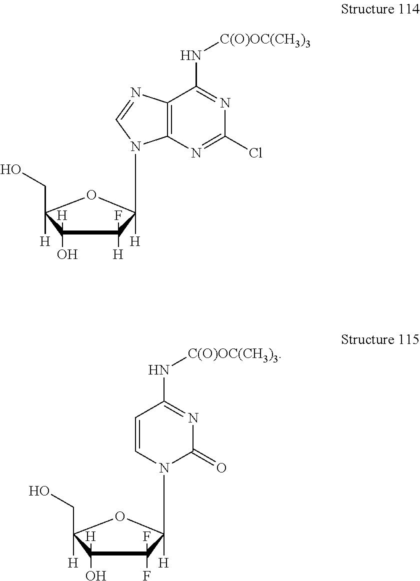

17. The therapeutically active nucleoside, a therapeutically active nucleoside analogue, a therapeutically active nucleotide, therapeutically active nucleotide analogue or therapeutic oligonucleotide according to claim 14, where it is selected from the following group: Fomivirsen including at least one thermally labile protecting group of the structure —C(O)OR⁸, where R⁸ is a tertiary alkyl group; and, Mipomersen including at least one thermally labile protecting group of the structure —C(O)OR⁸, where R⁸ is a tertiary alkyl group.

18. An oligonucleotide conjugate, wherein the oligonucleotide conjugate comprises one or more nucleotides or nucleotide analogues of the following structure: 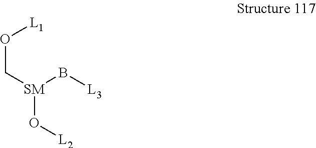
wherein the substituents of Structure 117 above are: L₁ and L₂ are independently H, a nucleotide, a nucleotide analogue, and a label, where there may be a linking group connecting the label to its position on the nucleotide or nucleotide analogue; L₃ is H, —C(O)OR⁶⁰ where R⁶⁰ is a tertiary alkyl, or a label, where there may be a linking group connecting the label to its position on the nucleotide or nucleotide analogue; and wherein if the label is not L₁, L₂ or L₃, it is attached to another nucleotide of the oligonucleotide; and wherein “SM” is a sugar moiety or an analogue of a sugar moiety; and wherein “B” is a nucleobase moiety or an analogue of a nucleobase moiety.

19. A method of synthesizing an oligonucleotide, wherein the method comprises the following steps:
1) coupling a compound to a solid support, either directly or through a linker, where the compound is of one of the following structures: 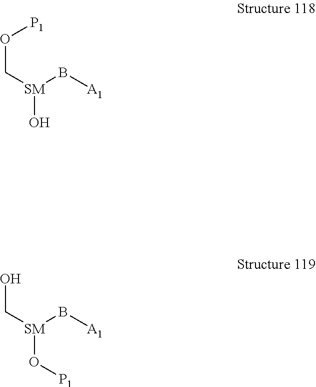
where “P₁” is a protecting group, “B” is a nucleobase or nucleobase analogue, “SM” is a sugar moiety or an analogue of a sugar moiety, and “A₁” is H or —C(O)OR⁴ where R⁴ is tertiary alkyl to provide a solid support compound of one of the following structures: 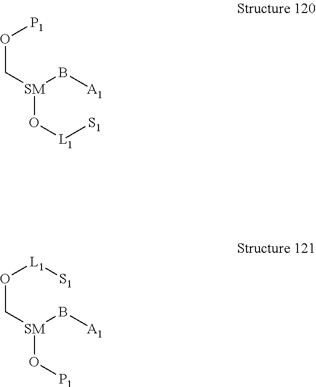
where L₁ is a linker or no chemical moiety, and S₁ is a solid support;
2) deprotecting the solid support compound to provide a deprotected compound of one of the following structures: 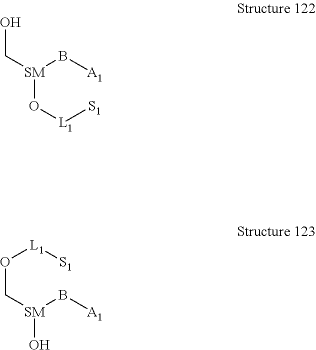
wherein L₁ is a linker or no chemical entity, and S₁ is a solid support; “B” is a nucleobase or nucleobase analogue, “SM” is a sugar moiety or an analogue of a sugar moiety, and “A₁” is H or —C(O)OR⁶⁰, where R⁶⁰ is a tertiary alkyl (e.g., —C(O)OC(CH₃)₃);
3) reacting the deprotected compound with a compound including a moiety comprising a phosphorus atom, wherein the compound is of one of the following structures: 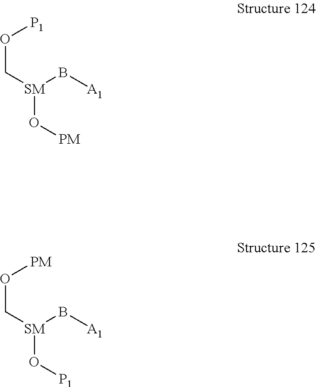
wherein “PM” is a phosphorus containing moiety, to provide a dinucleotide of one of the following structures; “P₁” is a protecting group (e.g., DMT); “B” is a nucleobase or nucleobase analogue; “SM” is a sugar moiety or an analogue of a sugar moiety; and “A₁” is H or —C(O)OR⁶⁰, where R⁶⁰ is a tertiary alkyl (e.g., —C(O)OC(CH₃)₃), to provide a dinucleotide of one of the following structures: 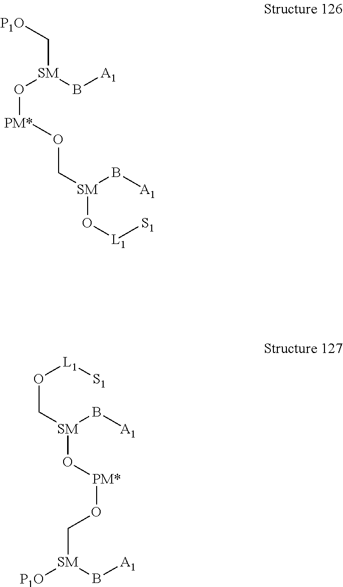
wherein “PM*” is the phosphorus containing moiety after the reaction, L₁ is a linker or no chemical entity, S₁ is a solid support, “P₁” is a protecting group, “B” is a nucleobase or nucleobase analogue, “SM” is a sugar moiety or an analogue of a sugar moiety, and “A₁” is H or —C(O)OR⁶⁰, where R⁶⁰ is a tertiary alkyl.
4) chemically modifying the phosphorus containing moiety to provide a modified dimer of one of the following structures: 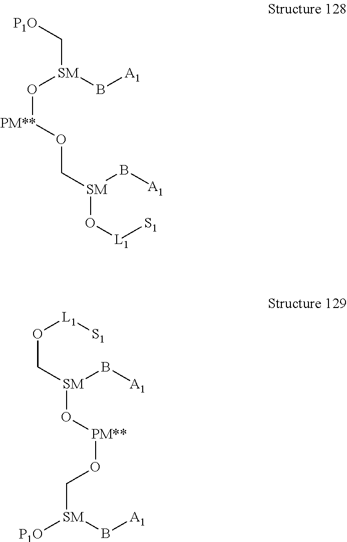
wherein “PM**” is a chemically modified phosphorus containing moiety, L₁ is a linker or no chemical entity, S₁ is a solid support, “P₁” is a protecting group, “B” is a nucleobase or nucleobase analogue, “SM” is a sugar moiety or an analogue of a sugar moiety, and “A₁” is H or —C(O)OR⁶⁰, where R⁶⁰ is a tertiary alkyl;
5) deprotecting the dimer or modified dimer to provide a deprotected dimer or modified dimer of one of the following structures: 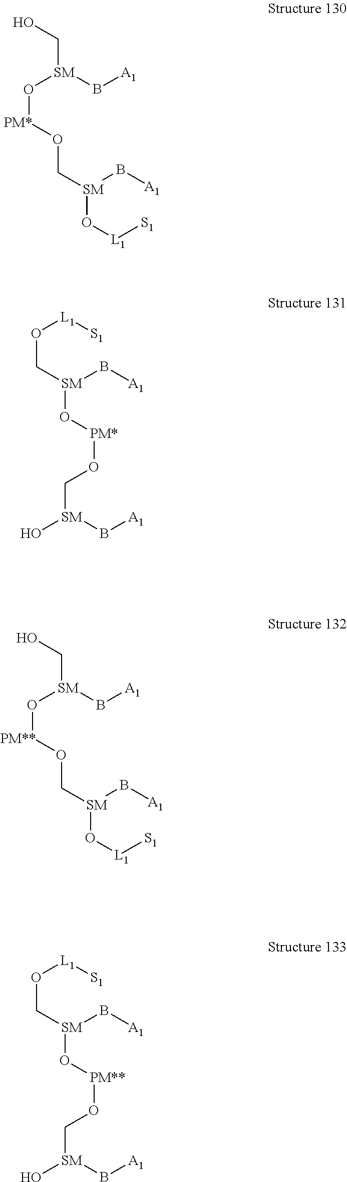
wherein “PM*” is the phosphorus containing moiety after the reaction to provide a dimer, “PM**” is a chemically modified phosphorus containing moiety, L₁ is a linker or no chemical entity, S₁ is a solid support, “B” is a nucleobase or nucleobase analogue, “SM” is a sugar moiety or an analogue of a sugar moiety, and “A₁” is H or —C(O)OR⁶⁰, where R⁶⁰ is a tertiary alkyl;
6) repeating steps “3” and “4” to provide an oligomer or modified oligomer of one of the following structures: 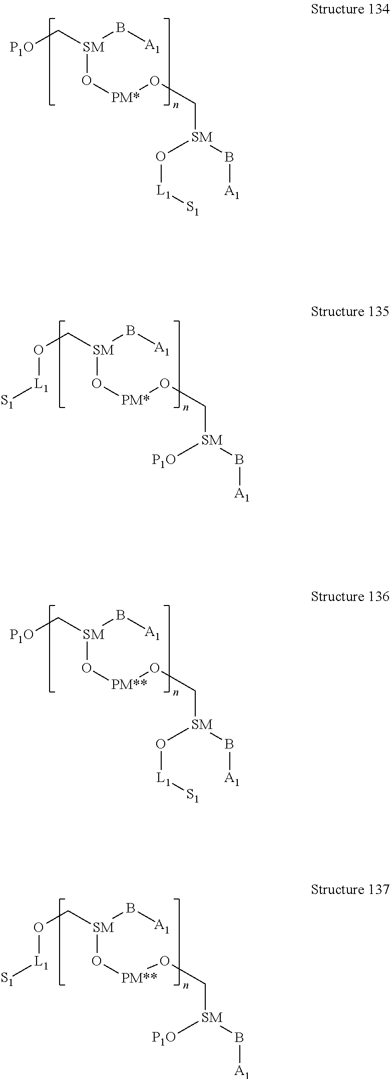
wherein “P₁” is a protecting group, “PM*” is the phosphorus containing moiety after the reaction to provide an oligomer, “PM**” is a chemically modified phosphorus containing moiety, “L₁” is a linker or no chemical entity, “S₁” is a solid support, “B” is a nucleobase or nucleobase analogue, “SM” is a sugar moiety or an analogue of a sugar moiety, and “A₁” is H or —C(O)OR⁶⁰, where R⁶⁰ is a tertiary alkyl; “n” is an integer ranging from 1 to 200;
7) deprotecting the dimer, modified dimer, oligonucleotide or modified oligonucleotide, removing it from the solid support, and chemically modifying the PM* or PM** moiety to provide a compound of the following structure: 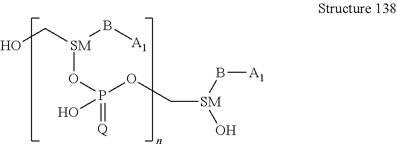
wherein “Q” is O or S, and where “n” is an integer ranging from 1 to 200, where at least one “A₁” is —C(O)OR⁶⁰, where R⁶⁰ is tertiary alkyl, “B” is a nucleobase or nucleobase analogue, and “SM” is a sugar moiety or an analogue of a sugar moiety.

20. The method according to claim 19, wherein the compound coupled to the solid is one of the following structures: 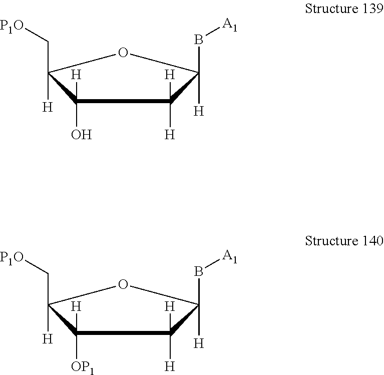
wherein “P₁” is a protecting group, “B” is a nucleobase or nucleobase analogue, and “A₁” is —H or —C(O)OR⁴, where R⁴ is tertiary alkyl.

21. The method according to claim 19, wherein the compound coupled to the solid is one of the following structures: 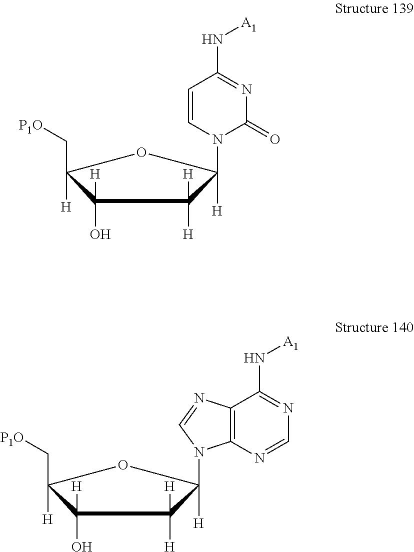
wherein “P₁” is a protecting group (e.g., DMT), and “A₁” is —H or —C(O)OR⁴, where R⁴ is tertiary alkyl; 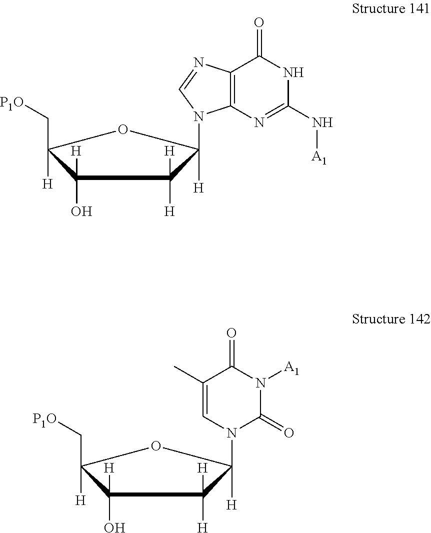
wherein “P₁” is a protecting group, and “A₁” is —H or —C(O)OR₄, where R₄ is tertiary alkyl.

22. The method according to claim 19, wherein the deprotected structure in step “2” is one of the following structures: 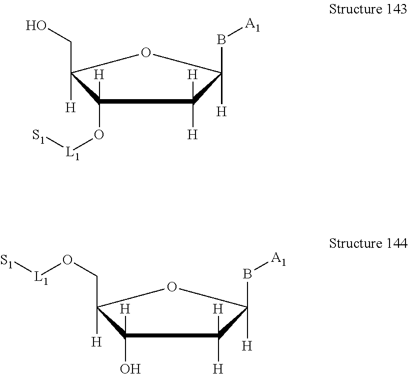
wherein “B” is a nucleobase or nucleobase analogue, “A₁” is —H or —C(O)OR⁴, where R⁴ is tertiary alkyl, L₁ is a linker or no chemical entity, and S₁ is a solid support.

23. The method according to claim 19, wherein the oligomer of step “7” is of the following structure: 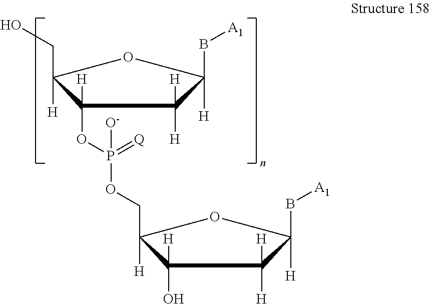
wherein “A₁” is —H or —C(O)OR⁶⁰, and where R⁶⁰ is tertiary alkyl, “B” is a nucleobase or nucleobase analogue, and “Q” is O or S.

24. A method of synthesizing an oligonucleotide, wherein the method comprises the following steps:
1) coupling a compound to a solid support, either directly or through a linker, where the compound is of one of the following structures: 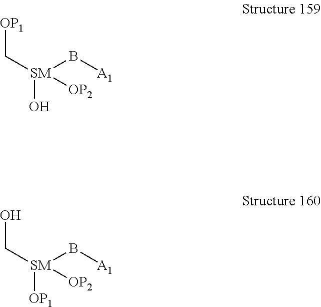
wherein “P₁” and “P₂” are independently protecting groups, “B” is a nucleobase or nucleobase analogue, “SM” is a sugar moiety or sugar moiety analogue, “As” is H or —C(O)OR⁶⁰ where R⁶⁰ is tertiary alkyl to provide a solid support bound compound of one of the following structures: 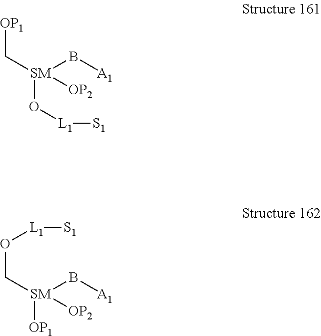
wherein “P₁” and “P₂” are independently protecting groups, “B” is a nucleobase or nucleobase analogue, “SM” is a sugar moiety or sugar moiety analogue, and “A₁” is H or —C(O)OR⁶⁰ where R⁶⁰ is tertiary alkyl, L₁ is a linker or no chemical moiety, and S₁ is a solid support;
2) deprotecting the solid support compound to provide a deprotected compound of one of the following structures: 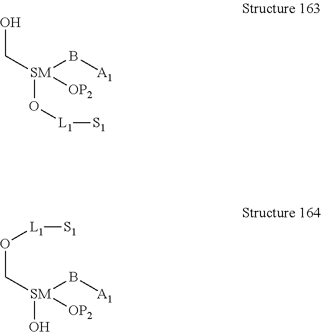
wherein “P₂” is a protecting group, “B” is a nucleobase or nucleobase analogue, “SM” is a sugar moiety or sugar moiety analogue, and “A₁” is H or —C(O)OR⁶⁰ where R⁶⁰ is tertiary alkyl, L₁ is a linker or no chemical moiety, and S₁ is a solid support;
3) reacting the deprotected compound with a compound including a moiety comprising a phosphorus atom, wherein the compound is of one of the following structures: 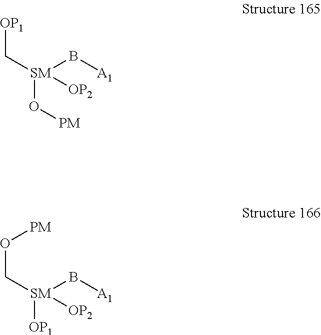
wherein “P₁” and “P₂” are independently protecting groups, “B” is a nucleobase or nucleobase analogue, “SM” is a sugar moiety or sugar moiety analogue, and “A₁” is H or —C(O)OR⁶⁰ where R⁶⁰ is tertiary alkyl (e.g., —C(O)OC(CH₃)₃)
to provide a dinucleotide of one of the following structures: 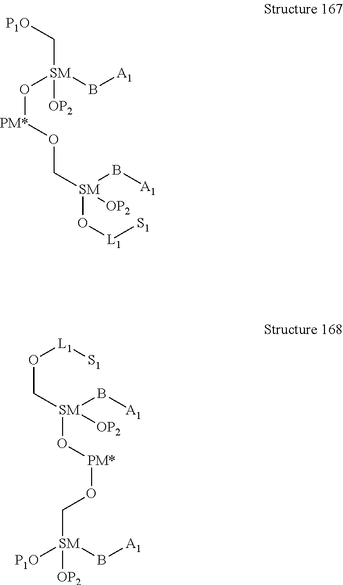
wherein “P₁” and “P₂” are independently protecting groups, “B” is a nucleobase or nucleobase analogue, “SM” is a sugar moiety or sugar moiety analogue, and “A₁” is H or —C(O)OR⁶⁰ where R⁶⁰ is tertiary alkyl, L₁ is a linker or no chemical moiety, and S₁ is a solid support;
4) chemically modifying the phosphorus containing moiety to provide a modified dimer of one of the following structures: 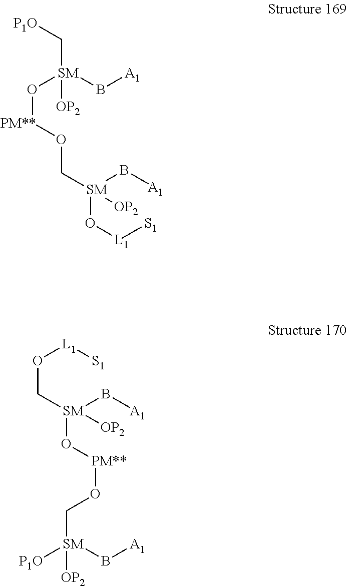
wherein “P₁” and “P₂” are independently protecting groups, “B” is a nucleobase or nucleobase analogue, “SM” is a sugar moiety or sugar moiety analogue, and “A₁” is H or —C(O)OR⁶⁰ where R⁶⁰ is tertiary alkyl, L₁ is a linker or no chemical moiety, and S₁ is a solid support;
5) deprotecting the dimer or modified dimer to provide a deprotected dimer or modified dimer of one of the following structures: 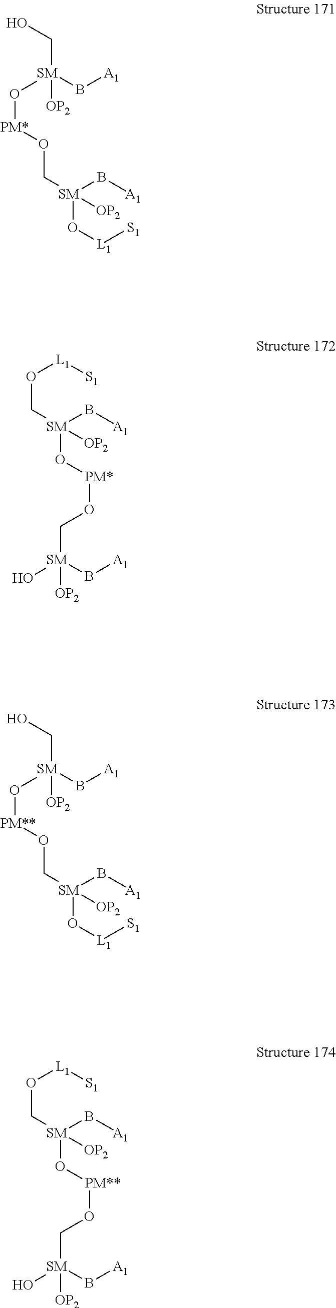
wherein “P₂” is a protecting group, “B” is a nucleobase or nucleobase analogue, “SM” is a sugar moiety or sugar moiety analogue, and “A₁” is H or —C(O)OR⁶⁰ where R⁶⁰ is tertiary alkyl (e.g., —C(O)OC(CH₃)₃), L₁ is a linker or no chemical moiety, and S₁ is a solid support, “PM*” is the phosphorus containing moiety after the coupling reaction, “PM**” is a chemically modified phosphorus containing moiety;
6) repeating steps “3” and “4” to provide an oligomer or modified oligomer of one of the following structures: 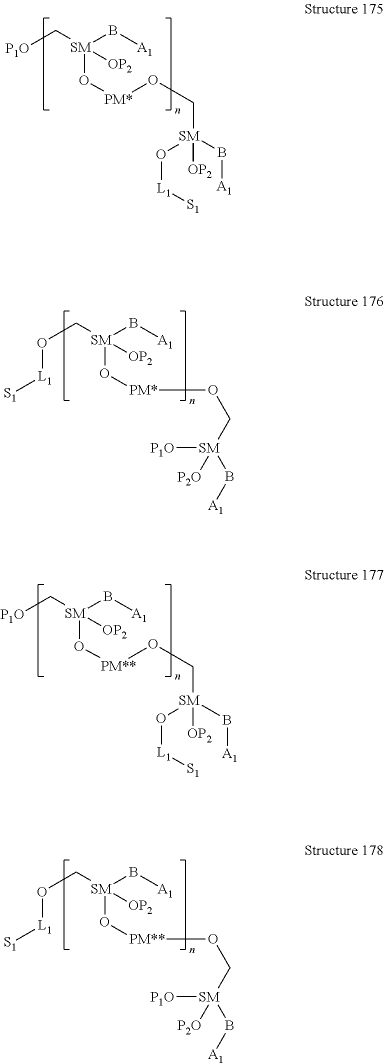
wherein “P₁” and “P₂” are, independently, protecting groups, “B” is a nucleobase or nucleobase analogue, “SM” is a sugar moiety or sugar moiety analogue, and “A₁” is H or —C(O)OR⁶⁰ where R⁶⁰ is tertiary alkyl (e.g., —C(O)OC(CH₃)₃), L₁ is a linker or no chemical moiety, St is a solid support, “PM*” is the phosphorus containing moiety after the coupling reaction, “PM**” is a chemically modified phosphorus containing moiety, “n” is an integer ranging from 1 to 200;
7) deprotecting the dimer, modified dimer, oligonucleotide or modified oligonucleotide, removing it from the solid support, and chemically modifying the PM* or PM** moiety to provide a compound of one of the following structures: 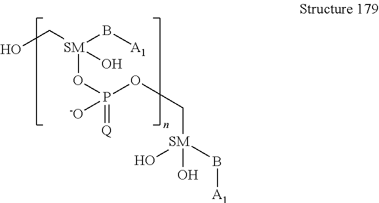
wherein “n” is an integer ranging from 1 to 200, “B” is a nucleobase or nucleobase analogue, “SM” is a sugar moiety or sugar moiety analogue, and where at least one “A₁” is —C(O)OR⁶⁰ where R⁶⁰ is tertiary alkyl, and where “Q” is O or S.

25. The method according to claim 24, wherein the oligomer in step “7” of the method is of the following structure: 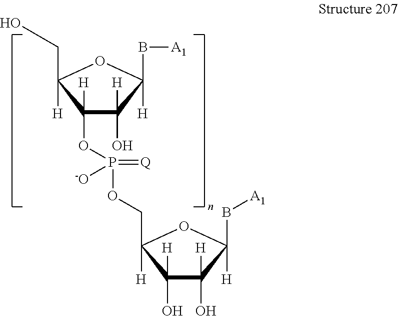
wherein “A₁” is —H or —C(O)OR⁶⁰, and where R⁶⁰ is tertiary alkyl, and “B” is a nucleobase or nucleobase analogue, and “Q” is O or S.

26. A method of amplifying DNA using the polymerase chain reaction (PCR), wherein the method comprises using one or more deoxynucleotide triphosphates having at least one thermally labile protecting group on a nitrogen atom on or within the ring structure of a nucleobase, where the protecting group is of the structure —C(O)OR⁴ where R⁴ is a tertiary alkyl.

27. The method according to claim 26, wherein the method comprises the following steps:
1) providing a reaction mixture comprising target DNA, DNA polymerase, primers and deoxynucleotide triphosphates (dNTPs), where one or more of the dNTPs is of one of the following structures: 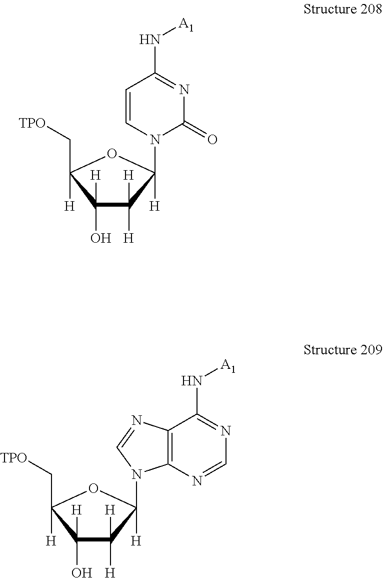
wherein “TP” is triphosphate, “A₁” is —C(O)OR⁶⁰, where R⁶⁰ is tertiary alkyl; 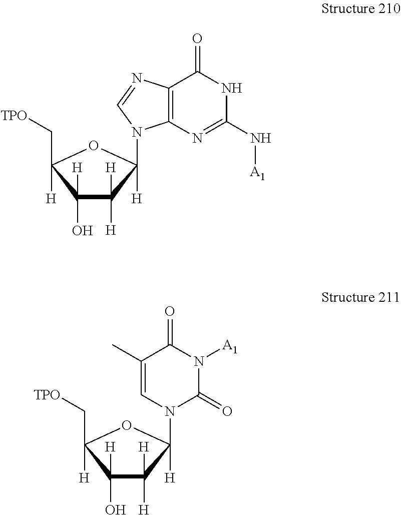
wherein “TP” is triphosphate, “A₁” is —C(O)OR⁶⁰, where R⁶⁰ is tertiary alkyl; 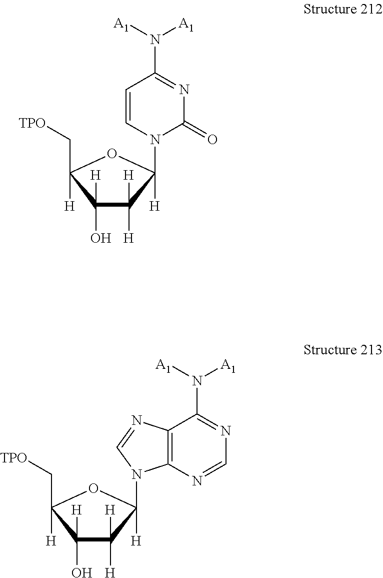
wherein “TP” is triphosphate, “As” is —C(O)OR⁶⁰, where R⁶⁰ is tertiary alkyl; 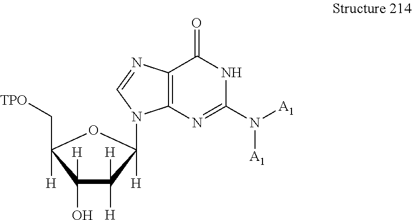
wherein “TP” is triphosphate, “A₁” is —C(O)OR⁶⁰, where R⁶⁰ is tertiary alkyl;
2) heating the reaction mixture for a period of time to denature the target DNA, thereby providing a single-stranded DNA template;
3) lowering the reaction temperature of the reaction mixture for a period of time which allows annealing of primers to the single-stranded DNA template to provide a primer-template complex and binding of the DNA polymerase to the primer-template complex;
4) heating the reaction mixture, allowing the DNA polymerase to synthesize a DNA strand complementary to the target DNA by adding the dNTPs to the DNA template in the 5′ to 3′ direction;
thereby amplifying DNA.

28. A method of amplifying DNA using the polymerase chain reaction, wherein the method comprises using one or more primers having one or more thermally labile protecting groups on a nitrogen atom on or within the ring structure of a nucleobase of the primer, where the protecting group is of the structure —C(O)OR⁴ where R⁴ is a tertiary alkyl.

29. The method according to claim 28, wherein the method comprises the following steps:
1) providing a reaction mixture comprising target DNA, DNA polymerase, primers and deoxynucleotide triphosphates, where one or more of the primers is of the following structure: 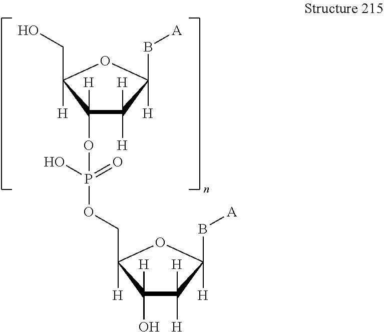
wherein “n” is an integer between 1 and 50, “B” is a nucleobase, and “A” is either H or a thermally labile protecting group of the structure —C(O)OR⁶⁰ where R⁶⁰ is tertiary alkyl, provided that at least one “A” is a thermally labile protecting group;
2) heating the reaction mixture for a period of time to denature the target DNA, thereby providing a single-stranded DNA template;
3) lowering the reaction temperature of the reaction mixture for a period of time, which allows annealing of primers to the single-stranded DNA template to provide a primer-template complex and binding of the DNA polymerase to the primer-template complex;
4) heating the reaction mixture, allowing the DNA polymerase to synthesize a DNA strand complementary to the target DNA by adding the dNTPs to the DNA template in the 5′ to 3′ direction;
thereby amplifying DNA.

30. A method of treating a disease in a patient, wherein the method comprises the following steps:
1) administering a compound to a patient in need thereof, wherein the compound comprises a nucleotide, nucleotide analogue, nucleoside or nucleoside analogue and one or more thermally labile protecting groups, where at least one of the thermally labile protecting groups is of the structure —C(O)OR⁸, and where R⁸ is a tertiary alkyl group;
2) applying thermal energy to one or more areas of the patient, resulting in the thermal deprotection of the nucleotide, nucleotide analogue, nucleoside or nucleoside analogue;
thereby treating the disease.

31. The method according to claim 30, wherein the administered compound is of one of the following structures: 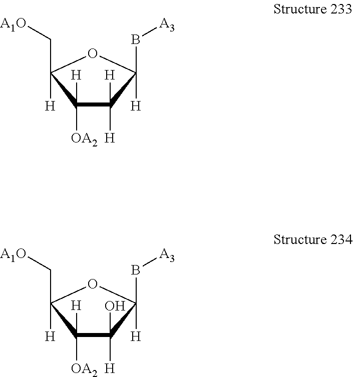
wherein “A₁”, “A₂” and “A₃” are, independently —H or a thermolabile protecting group, provided that at least one of A₁, A₂ or A₃ is a thermolabile protecting group of the structure —C(O)OR⁶⁰, where R⁶⁰ is a tertiary alkyl, and “B” is a nucleobase or nucleobase analogue.

32. A method of treating a disease in a patient, wherein the method comprises the following steps:
1) administering a compound to a patient in need thereof, wherein the compound comprises an oligonucleotide and one or more thermally labile protecting groups, where at least one of the thermally labile protecting groups is of the structure —C(O)OR, and where R⁸ is a tertiary alkyl group;
2) applying thermal energy to one or more areas of the patient, resulting in the thermal deprotection of the oligonucleotide;
thereby treating the disease.

33. The method according to claim 32, wherein the oligonucleotide is Fomivirsen or Mipomersen.

34. A method of making nucleoside or nucleoside analogue triphosphates, wherein the method comprises the steps of:
1) adding a monophosphorus reagent to a reaction mixture comprising a nucleoside or nucleoside analogue of the following structure: 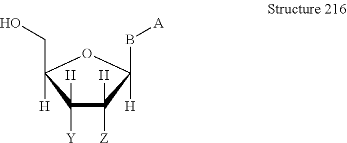
wherein Y is OP¹ where P′ is a protecting group, Z is H or OP² where P² is a protecting group, B is a nucleobase or a nucleobase analogue, and A is a thermally labile protecting group of the structure —C(O)OR⁶ where R⁶⁰ is a tertiary alkyl, to provide a mono-phosphorylated intermediate of the following structure: 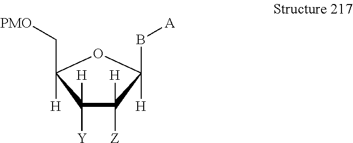
wherein Y is OP¹ where P¹ is a protecting group, Z is H or OP² where P² is a protecting group, B is a nucleobase or a nucleobase analogue, and A is a thermally labile protecting group of the structure —C(O)OR⁶ where R⁶⁰ is a tertiary alkyl, “PM” is a moiety comprising a single phosphorus atom;
2) adding a polyphosphorus reagent to the phosphorylated intermediate to provide a poly-phosphorylated intermediate of the following structure: 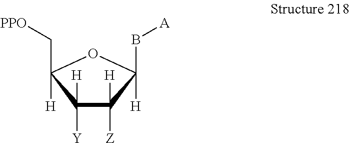
wherein Y is OP¹ where Pf1 is a protecting group, Z is H or OP² where P² is a protecting group, B is a nucleobase or a nucleobase analogue, A is a thermally labile protecting group of the structure —C(O)OR⁶⁰ where R⁶⁰ is a tertiary alkyl, and “PP” is a moiety comprising multiple phosphorus atoms;
3) hydrolyzing the poly-phosphorylated intermediate and removing P₁ to provide a nucleoside triphosphate or nucleoside analogue triphosphate of the following structure: 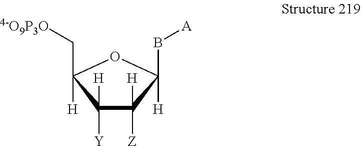
wherein Y is OP¹ where P¹ is a protecting group, Z is H or OP² where P² is a protecting group, B is a nucleobase or a nucleobase analogue, and A is a thermally labile protecting group of the structure —C(O)OR⁶⁰ where R⁶⁰ is a tertiary alkyl.

35. A method of deprotecting nucleosides, nucleoside analogues, nucleotides and nucleotide analogues, wherein the protected compounds are of the structure XO-SM-B-A, wherein “X” is H, a protecting group, a solid support, a phosphorus containing moiety or salts thereof, “SM” is a sugar moiety or an analogue of a sugar moiety, “B” is a base moiety of an analogue of a base moiety, “A” is one or more moieties attached to one or more nitrogen atoms on or within the base moiety and is of the structure —C(O)OR⁶⁰, wherein R⁶⁰ is a tertiary alkyl group, wherein the method comprises the step of:
heating the compound in the presence of a solvent having a pH greater than 7.0 to a temperature ranging from 90° C. to 100° C. for a period less than 45 minutes
thereby providing the deprotected compound.

36. A method of deprotecting an oligonucleotide of the structure 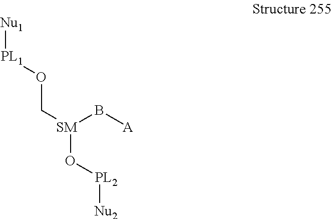
wherein “PL₁” and “PL₂” are, independently, either H or —P(O)(OH)O— or an analogue thereof, “Nu₁” and “Nu₂” are, independently, no substituent, a nucleoside or nucleoside analogue, or an oligonucleotide, “SM” is a sugar moiety or sugar moiety analogue, “B” is a nucleobase or nucleobase analogue, “A” is one or more moieties attached to one or more nitrogen atoms on or within the nucleobase moiety and is of the structure —C(O)OR⁶⁰, wherein R⁶⁰ is a tertiary alkyl group, wherein the method comprises the step of:
heating the compound in the presence of a solvent having a pH greater than 7.0 to a temperature ranging from 90° C. to 100° C. for a period less than 45 minutes thereby providing the deprotected compound.

37. A device for oligonucleotide synthesis, wherein the device comprises
a) one or more reservoirs containing chemical reagents used for synthesis of the oligonucleotide, wherein the reservoirs are operably connected in a system that allows flow of the various reagents to a synthesis chamber;
b) a mechanism to induce reagent flow to the synthesis chamber, where various chemical reactions involved in oligonucleotide synthesis are carried out;
wherein the synthesis chamber comprises an internal or external means to control its temperature.

38. The device according to claim 37, wherein the synthesis chamber comprises a synthesis column including a solid support, and wherein the means to control the temperature of the synthesis chamber controls the temperature of reagents in the synthesis chamber.

39. The device according to claim 38, wherein heating the reagents in the synthesis chamber induces deprotection of the oligonucleotides through removal of a group having a structure —C(O)OR⁶⁰, wherein R⁶⁰ is a tertiary alkyl group.

40. The device according to claim 39, wherein heating the oligonucleotides induces cleavage of the oligonucleotides from the solid support.
